(19) United States
(12) Patent Application Publication
(10) Pub. No.: US 2025/0145973 A1
(43) Pub. Date: May 8, 2025

(54) CONDITIONED DNA MODIFYING ENZYME COMPRISING HETEROLOGOUS DNA BINDING DOMAIN

(71) Applicant: Technische Universität Dresden Körperschaft des öffentlichen Rechts, Dresden (DE)

(72) Inventors: Liliya MUKHAMETZYANOVA, Dresden (DE); Frank BUCHHOLZ, Dresden (DE); Julia TORRES RIVERA, Berlin (DE)

(21) Appl. No.: 18/916,979

(22) Filed: Oct. 16, 2024

(30) Foreign Application Priority Data
Oct. 16, 2023 (EP) 23203894.3

(51) Int. Cl. C12N 9/12; A61K 38/00; C12N 15/10

(52) U.S. Cl. CPC C12N 9/1241; C12N 15/1034; A61K 38/00

(57) ABSTRACT

The present invention provides a method of identifying an amino acid position of a DNA modifying enzyme for insertion of a heterologous DNA binding domain (DBD), the method comprising the steps of providing a library of DNA modifying enzymes, wherein the members of the library comprise heterologous amino acid sequence insertions throughout the DNA modifying enzyme; identifying those DNA modifying enzymes of the library that have DNA modifying activity; and identifying the position of the insertion in those DNA modifying enzymes identified in the previous step. The present invention further pertains to methods of producing DNA modifying enzymes comprising an inserted heterologous DBD, to methods for modifying a nucleic acid sequence in a cell, and to methods for evolving a DNA binding domain on desired target sequences. Further provided are nucleic acid sequences encoding such DNA modifying enzymes and DNA binding domains, respective vectors and host cells.

### RELATED APPLICATIONS

This application claims priority to European Patent Application No. 23203894.3, filed Oct. 16, 2023, the entire disclosure of which is hereby incorporated herein by reference.

### SEQUENCE LISTING

The instant application contains a Sequence Listing which has been submitted electronically in XML format and is hereby incorporated by reference in its entirety. Said XML file, created on Oct. 14, 2024, is named 758598_TUD9-006_ST26.xml and is 270,084 bytes in size.

### FIELD OF THE INVENTION

The present invention relates to DNA modifying enzymes with conditioned activity conferred by insertion of a heterologous DNA binding domain. The present invention further pertains to methods of producing such DNA modifying enzymes, to methods for modifying a nucleic acid sequence in a cell, and to methods for evolving a DNA binding domain on desired target sequences. Further provided are nucleic acid sequences encoding such DNA modifying enzymes and DNA binding domains, respective vectors and host cells.

### BACKGROUND OF THE INVENTION

Tyrosine site-specific recombinases (Y-SSRs), such as the Cre-loxP system, are widely used genome editing tools, that hold great potential for therapeutic application due to their precise mechanism of DNA manipulation. Y-SSRs can execute complex genome engineering operations, including excision, inversion, integration, and cassette exchange of large genomic sequences, without inducing DNA double-stranded breaks. They also do not rely on the cellular DNA repair machinery or additional co-factors. Therefore, the DNA-editing process is predictable and works even in non-dividing cells (reviewed in Meinke et al., 2016). Despite these unique and useful properties, laborious step-wise directed molecular evolution and protein engineering are required for reprogramming them to target defined loci (Buchholz & Stewart, 2001; Sarkar et al., 2007; Buchholz & Hauber, 2011; Karpinski et al., 2016; Lansing et al., 2020; Lansing et al., 2022; Rojo-Romanos et al., 2023). This has limited the spread of recombinases as a versatile genome editing tool. Programmable nucleases, such as ZFNs, TALENs, and CRISPR-systems, on the other hand, can be easily programmed and adapted to different applications, which has laid the foundation for the genome editing revolution (Caroll, 2017; Anzalone et al., 2020; Wang & Doudna 2023). However, nucleases introduce DNA nicks or double-strand breaks, which require subsequent repair processes by the host cell, frequently resulting in undesired side effects (Kosicki et al., 2018; Adikusuma et al., 2018; Enache et al., 2020; Leibowitz et al., 2021; Papathanasiou et al., 2021; Sinha et al., 2021). Combining the precision of recombinases with the ease of targeting programmable nucleases would potentially provide best-in-class genome editing tools.

It is therefore an objective of the present invention to provide DNA modifying enzymes with conditioned activity and/or changed specificity.

### SUMMARY OF THE INVENTION

The objective underlying the present invention is solved by the provision of a method of identifying an amino acid position of a DNA modifying enzyme for insertion of a heterologous DNA binding domain (DBD), the method comprising the steps of:
(i) providing a library of DNA modifying enzymes, wherein the members of the library comprise heterologous amino acid sequence insertions throughout the DNA modifying enzyme;
(ii) identifying those DNA modifying enzymes of the library that have DNA modifying activity; and
(iii) identifying the position of the insertion in those DNA modifying enzymes identified in step (ii).

According to one embodiment, the one or more positions of the insertions identified in step (iii) are mapped to structural data of the DNA modifying enzyme.

According to a further embodiment, the library of DNA modifying enzymes provided in step (i) is encoded by a nucleic acid library.

According to a yet a further embodiment, step (iii) comprises determining at least part of the nucleic acid sequence encoding those DNA modifying enzymes identified in step (ii).

According to one embodiment, the method further comprises the step of selecting one or more amino acid positions for insertion of the heterologous DBD that are surface exposed in the DNA modifying enzyme and in proximity to the DNA binding site of the DNA modifying enzyme.

According to a further embodiment, the heterologous amino acid sequence comprised in each of the members of the library of DNA modifying enzymes independent of each other have a length of between three and ten amino acids, preferably a length of five amino acids.

According to another aspect, the present invention provides a method of producing a DNA modifying enzyme comprising an insertion of a heterologous DBD, the method comprising the steps of:
(i) inserting a nucleic acid sequence encoding the heterologous DBD into a nucleic acid sequence encoding the DNA modifying enzyme at the nucleotide triplet(s) encoding the one or more positions identified in the method of claims 1 to 6; and
(ii) expressing the nucleic acid sequence produced in step (i).

According to one embodiment, the nucleic acid sequence encoding the heterologous DBD further comprises a nucleic acid sequence encoding a peptide linker upstream and a peptide linker downstream of the nucleic acid sequence encoding the heterologous DBD. Preferably, the linker is a glycine-serine linker, more preferably a glycine-serine linker with at least one G to R substitution.

According to a preferred embodiment, the DBD is a zinc-finger (ZF) DBD or a transcription activator-like effector (TALE) DBD.

According to a further preferred embodiment, the DNA modifying enzyme is a transposase or a recombinase, preferably a serine recombinase or a tyrosine recombinase, more preferably a tyrosine recombinase.

According to yet another embodiment, the present invention provides a DNA modifying enzyme comprising an insertion of a heterologous DBD, which is obtained by the method of the present invention. The DNA modifying enzyme is preferably Cre or a Cre-derived recombinase and the DBD is inserted between amino acid positions 278 and 279. Alternatively, the DNA modifying enzyme is Vika or a Vika-derived recombinase and the DBD is inserted between amino acid positions 172 and 173. Alternatively, the DNA modifying enzyme comprises an amino acid sequence having at least 80% sequence identity to any one of SEQ ID NOs: 5, 9 to 13, 19, 20, 22, 30, 41, 42, 51, 64 and 91 to 200.

According to a further aspect, the present invention provides a DNA modifying enzyme comprising an insertion of a heterologous DBD, wherein the DBD optionally comprises at its N- and/or C-terminus a peptide linker, wherein the DNA modifying enzyme is inactive on its target site when the heterologous DBD does not bind to its target DNA, and wherein the DNA modifying enzyme is active on its target site when the heterologous DBD binds to its target DNA.

According to yet another aspect, the present invention provides a nucleic acid or a plurality of nucleic acids encoding the DNA modifying enzyme according to the invention.

According to a yet a further aspect, the present invention provides an expression vector comprising the nucleic acid or plurality of nucleic acids according to the invention.

According to a further aspect, the present invention provides a host cell or culture of host cells comprising the nucleic acid or plurality of nucleic acids of the invention, or the expression vector of the invention. Preferably, the host cell expresses the DNA modifying enzyme encoded by the nucleic acid or plurality of nucleic acids.

According to yet another aspect, the present invention provides a pharmaceutical composition comprising the DNA modifying enzyme of the invention, the nucleic acid or plurality of nucleic acids of the invention, the expression vector of the invention, or the host cell or culture of host cells of the invention, and a pharmaceutically acceptable excipient or carrier.

According to a further aspect, the present invention provides the use of the DNA modifying enzyme of the invention, of the nucleic acid or plurality of nucleic acids of the invention, of the expression vector of the invention, of the host cell or culture of host cells of the invention, or of the pharmaceutical composition of the invention, for modifying a nucleic acid sequence of interest.

According to yet another aspect, the present invention provides a method for modifying a nucleic acid sequence of interest, comprising contacting a cell or tissue comprising the nucleic acid sequence of interest with the DNA modifying enzyme of the invention, the nucleic acid or plurality of nucleic acids of the invention, the expression vector of the invention, the host cell or culture of host cells of the invention, or the pharmaceutical composition of the invention under conditions allowing the DNA modifying enzyme to modify the nucleic acid sequence of interest.

According to a further aspect, the present invention provides a method of changing the specificity and/or activity of a DNA modifying enzyme comprising the steps of:
(i) identifying an amino acid position of a DNA modifying enzyme for insertion of a heterologous DBD according to the invention; and
(ii) inserting a heterologous DBD at the position identified in step (i).

According to yet another aspect, the present invention provides a method of evolving a DBD on a target sequence of interest, comprising the steps of:
(i) creating a library of variants of the DBD;
(ii) cloning the library of step (i) into expression vectors comprising a first region encoding a DNA recombining enzyme, a second region comprising a first target site of said DNA recombining enzyme and regions flanking said first target site, and a third region comprising a second target site of said DNA recombining enzyme and regions flanking said second target site, such that a DBD is inserted directly or via peptide linkers into the DNA recombining enzyme, wherein the first, second and third regions are separated from another;
(iii) introducing the expression vectors into host cells and culturing the host cells, thereby expressing the encoded DNA recombining enzyme comprising the DBD;
(iv) isolating plasmids from the cell culture of step (iii) and determining whether the DNA recombining enzyme catalyzed a recombination reaction at both target sites on the vector;
(v) amplifying the DBD of those plasmids that were found to encode a DNA recombining enzyme comprising the DBD showing recombination activity using error-prone PCR to generate a new library of variants of the DBD;
(vi) repeating steps (ii) to (iv) with the library of step (v).

Further aspects and embodiments of the invention will become apparent from the appending claims and the following detailed description.

According to a further aspect, the present invention provides a DNA modifying enzyme comprising a heterologous DNA binding domain inserted therein, wherein the protein comprises an amino acid sequence having at least 80% sequence identity to any one of SEQ ID NOs: 5, 9 to 13, 19, 20, 22, 30, 41, 42, 51, 64 and 91 to 201.

Further aspects and embodiments are derivable from the following detailed description, examples and the figures.

### DESCRIPTION OF THE DRAWINGS

The invention is further illustrated by the following figures and examples without being limited thereto.

FIGS. 1A-1C schematically show constructs used in the present invention. FIG. 1A is a schematic representation of the plasmid constructs used for the recombination assay in E. coli. The recombinase or Recombinase-DBD fusion complex is expressed under arabinose-inducible promoter, which allows to obtain different levels of protein expression in the cells. The lox- or lox-if sites are indicated as triangles. Upon recombination by the expressed variant, the DNA fragment between two target sites is excised resulting in a smaller plasmid. FIG. 1B is a schematic representation of the plasmid constructs used for the recombination assay in HEK293T cells. Important elements of the expression and reporter plasmids are indicated. Nuclear localization signal (NLS), the enhanced green fluorescent protein (EGFP) sequence and the internal ribosome site (IRES) are shown. Upon recombination of the loxBTR or loxBTR-5-zif target sites on the reporter, the 3×SV40poly(A) is excised allowing for the expression of the red fluorescent protein (mCherry). FIG. 1C shows a schematic representation of the plasmid constructs used for transfection of HEK293T cells. Important elements of the expression plasmids are indicated. In the expression plasmids, the D7L recombinase or the D7L-ZFL recombinase is fused to a nuclear localization signal (NLS), and to the TagBFP via a P2A; the D7R recombinase or the D7R-ZFR recombinase is fused to an NLS, and to EGFP via a P2A.

FIGS. 2A-2B show overviews of assays for determining activity of recombining enzymes. FIG. 2A is a schematic representation of the PCR-based recombination test that was used for analysis of single clones (adapted from Lansing et al. 2022). Primers are indicated as P1, P2, and P3. Three-primer PCR generates a bigger fragment (491 bp) from the non-recombined pEVO plasmid, or a smaller fragment (400 bp) from the recombined pEVO plasmid, or if a mix of both plasmids used for the PCR both bands can be detected. The upper band represents the unrecombined plasmid (line with two triangles), and the lower band represents the recombined plasmid (line with one triangle). FIG. 2B is a schematic of the plasmid-recombination assay. An example of the wt recombinase tested in the pEVO plasmid is shown. The recombination of pEVO plasmid by the expressed recombinase results in excision of the DNA fragment between two lox-sites, leading to reduction of the plasmid size. Upon a restriction enzyme digest of the plasmids, and excision of the 1 kb recombinase gene (in the example shown), the difference between the recombined (line with one triangle) and non-recombined plasmids (line with two triangles) can be detected by agarose gel electrophoresis. “Mix” represents a mixture of the recombined and non-recombined plasmids, the ratio between which can be used to calculate the recombination efficiency. M=Marker.

FIG. 3 is a schematic representation of the pentapeptide scanning mutagenesis procedure. The recombinases (Cre, D7L, D7R, Brec1) cloned into the pEVO vector were combined with the MuA transposase and Entranceposon for the transposition reaction that resulted in the library, in which the Entranceposon was randomly inserted within the recombinase sequence. In the next step, the inserted Entranceposon was excised by a restriction enzyme digest, resulting in the library of recombinase mutants carrying five amino acid insertions. The obtained library was cloned into pEVO plasmids carrying the respective recombinase target sites for selection. Upon expression of the recombinases, the mutated variants retaining recombination activity on the target sites were selected by digestion with a unique restriction enzyme (depicted as scissors) and sequenced with PacBio long-read sequencing.

FIG. 4 shows the results from the pentapeptide scanning mutagenesis screens. Frequencies and distribution of the five amino acids insertions in the sequences of active Brec1, D7L, D7R and Cre recombinases are shown. Secondary structure elements are indicated, with alpha-helices displayed as cylinders with letters and beta-sheets represented as numbered arrows, according to the secondary structure of Cre (Meinke et al., 2016).

FIG. 5 shows cumulative frequencies of insertions for Brec1, D7L, D7R and Cre. The top 5 residues with the highest insertion frequencies are indicated with their amino acid positions.

FIGS. 6A-6B show a 3D drawing of a Cre-type recombinase and positions identified therein for pentapeptide insertions. FIG. 6A shows the dimer of the Cre/loxP synapse pre-cleavage complex (PDB ID 1Q3U, Ennifar E. et al., 2003). The DNA is shown in black, and the two Cre monomers are shown in grey-scale. The most frequent positions that tolerated insertions in the pentapeptide scanning mutagenesis of the Cre-type recombinases are highlighted and indicated by arrows with positions numbered. FIG. 6B shows a monomer of the Cre/loxP synapse pre-cleavage complex (PDB ID 1Q3U, Ennifar E. et al., 2003). The distances between selected residues and the DNA (to the nucleotide following the loxP target site) are indicated.

FIG. 7 shows prediction and analysis of the 3D protein structure of Vika recombinase. The 3D model of Vika wt was predicted using AlphaFold (Mirdita et al., 2022). The predicted model is superimposed with the monomer of the Cre/loxP synapse pre-cleavage complex (PDB ID 1Q3U, Ennifar E. et al., 2003). The most frequent positions that tolerated insertions in the pentapeptide scanning mutagenesis of the Vika-type recombinase are highlighted and the amino acid is indicated. The distances between the selected residues and the DNA (to the nucleotide following the lox target site) are indicated. The image was created using the 3D Protein imager (Tomasello et al., 2020).

FIGS. 8A-8B show the insertional fusion of a heterologous zinc-finger domain to Brec1 recombinase and target site orientations. FIG. 8A shows a schematic representation of an insertional fusion of Zif268 DNA binding domain flanked with flexible linkers of 1 to 8 Gly-Gly-Ser repeats, inserted into the Brec recombinase sequence between the residues 278 and 279. FIG. 8B is an overview of the target site library. Important features are indicated, bp=base pair.

FIG. 9 is a schematic presentation of the plasmid recombination screen comprising nanopore sequencing. Recombinase and recombinase target sites (triangles) are shown in dark grey. Zif268 and its target sites (ellipses) are shown in black, linkers are presented in light grey. R1=restriction site 1; R2=restriction site 2.

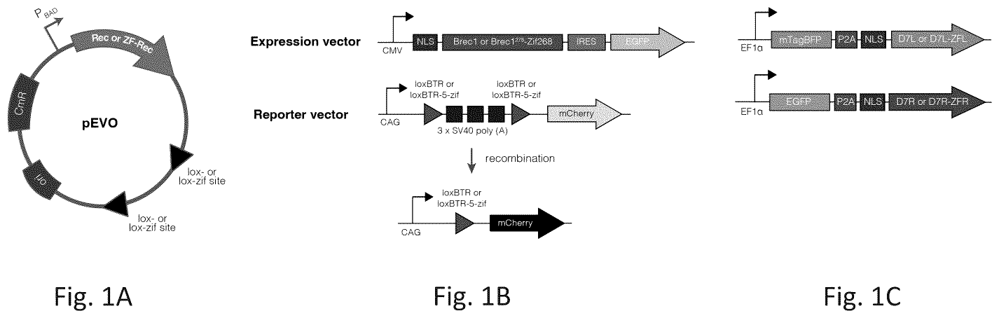

FIGS. 1A-10B show activities of insertional fusion proteins in E. coli. FIG. 10A shows the results of a plasmid-based activity assay for the indicated variants of the DNA modifying enzyme (here recombinase Brec1) and target sites at high induction level (200 μg/ml L-arabinose). The upper band represents the unrecombined plasmid (line with two triangles), and the lower band represents the recombined plasmid (line with one triangle). M=Marker. FIG. 10B shows quantified results of the recombination rates for indicated variants in bacteria. Recombination efficiencies were calculated from band intensities shown as in FIG. 10A. The assay was performed in triplicates (n=3), plotted as dots, the bar graphs represent mean values, the error bars indicate the standard deviation from the mean. Statistical relevance of the triplicates was assessed using a two-sample t-test. (ns): P>0.5, (*): P≤0.05, (****): P≤0.0001.

FIG. 11 shows the recombination efficacy of insertional fusion proteins in human cells. The quantification of recombination efficiencies in HEK293T cells 48h post transfection for the indicated DNA modifying enzyme variants is shown, analysed by flow cytometry. Samples transfected only with reporter plasmids were used as a control. Recombination rates were calculated as the percentage of the recombined cells (mCherry positive) normalized for transfection efficiency (GFP positive). The assay was performed in triplicates (n=3), plotted as dots, the bar graphs represent mean values, the error bars indicate the standard deviation from the mean. Statistical relevance of the triplicates was assessed using a two-sample t-test. (ns): P>0.5, (****): P≤0.0001.

FIG. 12 shows recombination efficacy of insertional fusion in different Vika-based DNA modifying enzymes. FIG. 12 shows results of a plasmid-based activity assay for the created DNA modifying enzymes with insertion of a heterologous DBD. Zif268 was fused between residues 172 and 173 ((GGS)₈×(GGS)₈ linkers were used for all fusions) of Vika2, Vika3, and Vika4 (Vika4 was not part of the pentapeptide scanning mutagenesis screen). The activities on the respective recombinase target sites (vox2, vox3, vox4) and their vox-5-zif (vox2-5-zif, vox3-5-zif, vox4-5-zif) (A) and (B) versions were tested. The test was performed at 100 μg/ml L-arabinose. Activity of the wild-type recombinases, without Zif268 fusions (Vika2, Vika3, Vika4) is shown as a control. The upper band represents the unrecombined plasmid (line with two triangles), and the lower band represents the recombined plasmid (line with one triangle). M=Marker. Quantification of the recombination rates for indicated variants and target sites is shown on the right side. Recombination efficiencies were calculated from ratios of recombined and non-recombined gel band intensities shown on the left side. The assay was performed in triplicates (n=3), plotted as points, the bar graph represents the mean value, the error bars indicate the standard deviation from the mean. Statistical relevance of the triplicates was assessed using a two-sample t-test. (ns): P>0.5, (*): P≤0.05, (**): P≤0.01, (***): P≤0.001, (****): P≤0.0001.

FIGS. 13A-13D show activity tests of recombinases rendered conditional by insertional fusion of a zinc-finger domain. FIG. 13A shows the results of the plasmid-based activity assay for Brec1²⁷⁸-ZFCCR5L, in which ZifCCR5L (Perez et al., 2008) is fused between the residues 278 and 279 of Brec via (GGS)₈ linkers. The fusion complex was tested on loxBTR, loxBTR-5-zifCCR5L (A), or loxBTR-5-zifCCR5L (B) target sites. The test was performed at 200 μg/ml L-arabinose. Activity of the wild-type Brec is shown as a control. FIG. 13B shows the results of the plasmid-based activity assay of RecHTLV²⁷⁸-Zif268 fusion complex, in which Zif268 is fused between the residues 278 and 279 of RecHTLV via (GGS)₈ linkers. The fusion complex was tested on loxHTLV, loxHTLV-5-zif268 (A), or loxHTLV-5-zif268 (B) target sites. The test was performed at 100 μg/ml L-arabinose. Activity of the wild-type RecHTLV is shown as a control. FIG. 13C shows the results of the plasmid-based activity assay for D7L²⁷⁸-Zif268, in which Zif268 is fused between the residues 278 and 279 of D7L via (GGS)₈ linkers. The fusion complex was tested on loxF8L, loxF8L-5-zif268 (A), or loxF8L-5-zif268 (B) target sites. The test was performed at 10 μg/ml L-arabinose. Activity of the wild-type D7L is shown as a control. FIG. 13D shows the results of the plasmid-based activity assay for D7R²⁷⁸-Zif268, in which Zif268 is fused between the residues 278 and 279 of D7R via (GGS)₈ linkers. The fusion complex was tested on loxF8R, loxF8R-5-zif268 (A), or loxF8R-5-zif268 (B) target sites. The test was performed at 200 μg/ml L-arabinose. Activity of the wild-type D7R is shown as a control. For FIGS. 13A-13D the upper band represents the unrecombined plasmid (line with two triangles), and the lower band represents the recombined plasmid (line with one triangle). M=Marker.

FIGS. 14A-14C show that the target site specificity of a DNA modifying enzyme can be manipulated by an insertional fusion with a DNA binding domain. FIG. 14A shows an alignment of target sites recombined by the RecFlex recombinase. A position probability matrix for the left and the right half-sites is shown. A search using this matrix for potential therapeutic human genomic target sites revealed a candidate site on chromosome X (loxMECP2; SEQ ID NO: 29). FIG. 14B shows the target sites used for the activity assay. Activity of RecFlex and RecHex²⁷⁸-Zif268 was tested on two types of target sites: loxFlex (34 bp) (SEQ ID NOs: 24 to 29) and the loxFlex-5-zif target sites (SEQ ID NOs: 85 to 90), where loxFlex is flanked by the Zif268 binding motif (9 bp) in orientation B (as shown in the figure) relative to the lox-site and spaced by the 5 base pairs. FIG. 14C shows the results of a plasmid-based activity assay of RecFlex and RecFlex²⁷⁸-Zif268. Activity was tested on the five loxFlex sites and on the human genomic loxMECP2 target site, as well as on their lox-zif versions, in which the loxFlex sites were flanked by the Zif268 binding motifs (spaced by the 5 bp, as shown in (b)). The upper band represents the unrecombined plasmid (line with two triangles), and the lower band represents the recombined plasmid (line with one triangle). The assay was performed in triplicates (n=3). Recombination efficiencies were calculated from the ratio of the recombined and non-recombined band intensities, with mean recombination efficiencies indicated underneath the gel picture. M=Marker.

FIGS. 15A-15B show the design of zinc-finger domains for the loxF8 genomic locus. FIG. 15A shows the genomic sequences flanking loxF8. Binding sites of the designed ZFDs upstream (ZFL1-2) and downstream (ZFR1-4) are shown with the distance (bp) between the loxF8 and the ZF motifs indicated. FIG. 15B shows the results of a plasmid-based activity assay of the monomers from the D7 recombinase heterodimer (D7L and D7R) fused with the designed ZFDs on the symmetric loxF8L and loxF8R target sites and their extended versions that include the respective flanking genomic sequences for ZFD binding (loxF8L-flank and loxF8R-flank). The activity of the wild-type monomers is shown as a control. High induction levels (100 μg/ml L-arabinose for D7L and D7L-ZFL, 200 μg/ml L-arabinose for D7R and D7R-ZFR) were used to show that even at high expression level of the proteins, the fusion complexes are essentially inactive on the lox-sites without the zif motifs, while the recombinases alone recombine these sites with high efficiency. The upper band represents the unrecombined plasmid (line with two triangles), and the lower band represents the recombined plasmid (line with one triangle). M=Marker. Fusions with activity (D7L-ZFL1 (SEQ ID NO: 91) and D7R-ZFR4 (SEQ ID NO. 92)) are highlighted with boxes.

FIG. 16 is a schematic presentation of substrate-linked directed molecular evolution for zinc finger domains (ZF-SLiDE). Evolution cycles start with cloning the ZFD library between residues 278 and 279 of the recombinase sequence encoded in the pEVO vector. Additionally, the vector carries two lox-zif target sites of interest (lox-sites are indicated as triangles, zif motifs are indicated as ellipses flanking the lox-sites). After expression of the ZFD-recombinase fusion, plasmid DNA is isolated and analysed. Upon successful recombination, a unique restriction site (indicated as scissors) between two target sites is excised. By applying restriction digestion, the non-recombined plasmids are linearized, while the recombined plasmids remain circular. The digestion is followed by a PCR using indicated primers (arrows), which generates product only from recombined plasmids. Successful ZF variants are then subjected to the next round of directed evolution. Counter-selection is applied with vectors containing the lox-sites of interest alone, without the zif motifs.

FIGS. 17A-17B show the results of the substrate-linked directed molecular evolution for zinc finger domains. FIG. 17A shows the ZF-SLiDE progress assessed by plasmid-based recombination assay. Recombination efficiency of D7L and D7R fused with the ZF libraries on the loxF8L-flank and loxF8R-flank target sites, respectively, are shown at the start of directed evolution and after the 20 cycles of ZF-SLiDE. Activities of the final libraries on the loxF8L and loxF8R sites are shown as a control. All tests were performed at high induction level (200 μg/ml L-arabinose). M=Marker. FIG. 17B shows the sequence analysis of the evolved ZFDs obtained by sequencing 78 and 59 active variants from the final ZFL and ZFR libraries, respectively. The frequency of the mutated residues compared to the designed ZFD that served as a starting point (ZFL1 and ZFR4) are shown. The number of different amino acids identified at particular positions is indicated in the bar graph. The core helices of the ZFDs are highlighted. The most conserved mutations are indicated by the residue number and the amino acid substitution.

FIG. 18 shows that a mutation in the linker improves recombination efficiency. The figure shows a comparison of plasmid-based activity assays for Brec1²⁷⁸-Zif268 containing a mutation in the right linker (Brec1-(GGS)₈-Zif268-(GGS)₆-GRS-GGS) (SEQ ID NO: 64) and Brec1²⁷⁸-Zif268 (Brec1-(GGS)₈-Zif268-(GGS)₈) (SEQ ID NO: 5) on the loxBTR (SEQ ID NO: 65) and loxBTR-5-zif (A) (SEQ ID NO: 66) target sites. The test was performed at 100 μg/ml L-arabinose. Activity of the wild-type Brec1 is shown as a control. Recombination efficiencies were calculated from ratios of recombined and non-recombined band intensities. The upper band represents the unrecombined plasmid (line with two triangles), and the lower band represents the recombined plasmid (line with one triangle). M=Marker.

FIG. 19 shows a mutational analysis of D7-ZF (a dimer comprising D7L-ZFL (SEQ ID NO: 41) and D7R-ZFR (SEQ ID NO: 42)) and the flanking linkers (one-letter code). The amino acid sequence of ZFL1 (SEQ ID NO: 31) and ZFR4 (SEQ ID NO: 36) and the flanking (GGS)₈ linkers are shown as a reference. Dots indicate conserved residues.

FIGS. 20A-20C show improved properties of D7-ZF over D7. FIG. 20A shows plasmid-based activity assay of D7-ZF and D7 on the loxF8 and its extended version (loxF8-flank) that includes the flanking genomic sequences for the ZFD binding target sites as found in the human genome. FIG. 20B shows plasmid-based activity assay of D7-ZF on predicted human genomic loxF8-like off-targets that are recombined by D7, and their extended versions that include the flanking genomic sequences upstream and downstream of the lox-sites. FIG. 20C shows plasmid-based activity assay of D7 and D7-ZF on predicted human genomic loxF8-like off-targets flanked with the sequences potentially recognized by D7-ZF. For FIGS. 20A-20C, tests were performed at high induction level (100 μg/ml L-arabinose); the upper band represents the unrecombined plasmid (line with two triangles), and the lower band represents the recombined plasmid (line with one triangle); M=Marker.

FIGS. 21A-21E show a schematic representation of a fraction of the F8 gene (FIG. 21A), agarose gel images of PCR products, and quantification of inversion efficiencies by D7-ZF. FIG. 21A displays the two possible orientations of the loxF8 locus (wild-type and inverted orientation) (adapted from Lansing et al., 2022). Primers used for PCR to detect the orientation of the locus are indicated as P1, P2, P3. The position of the loxF8 sites and the distance between them are indicated. The transcription start site of the F8 gene is depicted by a black arrow. FIG. 21B shows the agarose gel image of PCR products generated using the indicated primer combinations to detect the orientation of the loxF8 locus in the F8 gene of the HEK293T cells 72 h post transfection with D7 or D7-ZF. The non-treated HEK293T cells were used as a wild-type control, the iPSCs derived from a patient carrying the Exon1 inversion were used as an inversion control. Primer combinations are indicated. M=Marker. FIG. 21C shows inversion efficiencies of the loxF8 locus in HEK293T cells 72 hours post transfection with D7 and D7-ZF, quantified by qPCR. The assay was performed in triplicates (n=3), plotted as dots. The bar graphs represent mean values, the error bars indicate the standard deviation from the mean. Statistical relevance of the triplicates was assessed using a two-sample t-test. (**): P<0.01. FIG. 21D shows an agarose gel image of PCR products generated using the indicated primer combinations to detect the orientation of the loxF8 locus in the F8 gene of the patient-derived F8 hiPSCs cells 48 h post transfection with D7 or D7-ZF mRNA. The cells treated with GFP mRNA were used as a wild-type control, the wild-type hiPSCs derived from a healthy donor were used as an inversion control. The experiment was done is triplicates (n=3). Primer combinations are indicated. M=Marker. FIG. 21E shows fold-increase of D7-ZF inversion efficiency in patient-derived F8 hiPSCs over D7, quantified by qPCR.

FIGS. 22A-22B show activity of Brec1²⁷⁸-Zif268 in view of the number of zif motifs. FIG. 22A presents the tested target sites where the loxBTR sites are flanked by different number of zif-motifs. On the pEVO, two loxBTR sites are present, therefore, the maximum number of zif-motifs is four. FIG. 22B shows plasmid-based activity assay for Brec1²⁷⁸-Zif268 fusion complex on the loxBTR-5-zif target sites with different number of zif motifs, that are shown in FIG. 22A. Activity of Brec1 is shown as a control. The test was performed at high induction level (200 μg/ml L-arabinose). The upper band represents the unrecombined plasmid (line with two triangles), and the lower band represents the recombined plasmid (line with one triangle). M=Marker.

FIGS. 23A-23B show the activity of a DNA modifying enzyme with inserted TAL domain. FIG. 23A depicts the target sites used for the activity assay of Brec1²⁷⁸-TAL2295. Recombination efficiency was tested on two types of target sites: recombinase loxBTR target site only, or the loxBTR-5-TAL2295 target sites (Reyon et al., 2012) where the loxBTR is flanked by the TAL2295 binding site in two different orientations (A and B) relative to each other and at a distance of 5 bp between the binding sites. FIG. 23B shows the results of a plasmid-based activity assay for Brec1²⁷⁸-TAL2295 fusions on loxBTR and loxBTR-5-TAL2295 (A and B). Activity of the wild-type Brec is shown as a control. The test was performed at high induction level (200 μg/ml L-arabinose). The upper band represents the unrecombined plasmid (line with two triangles), and the lower band represents the recombined plasmid (line with one triangle). M=Marker.

FIG. 24 shows the results from the pentapeptide scanning mutagenesis screens. Frequencies and distribution of the five amino acids insertions in the sequences of active Bxb1 and evolved variants thereof are shown. Secondary structure elements are indicated, with alpha-helices displayed as cylinders with letters and beta-sheets represented as numbered arrows, according to the secondary structure of Bxb1 (Van Duyne and Rutherford, 2016).

FIG. 25 shows cumulative frequencies of insertions for Bxb1 and evolved variants. The top 4 residues with the highest insertion frequencies are indicated with their amino acid positions.

FIG. 26 shows prediction and analysis of the 3D protein structure of Bxb1 recombinase. The 3D model of Bxb1 wildtype was predicted using AlphaFold (Mirdita et al., 2022). The predicted model is superimposed with the C-terminal domain of a serine integrase A118 (SEQ ID NO: 202) bound to an attP DNA half-site (attP(A118) (SEQ ID NO: 217), PDB ID 4KIS (Rutherford, 2013)). The most frequent positions that tolerated insertions in the pentapeptide scanning mutagenesis of the Bxb1-type recombinase are highlighted. Distances between the selected residues and the DNA (to the nucleotide following the attP target site) are also shown. The image was created using the 3D Protein imager (Tomasello et al., 2020).

FIGS. 27A-27D show recombination efficacy of insertional fusion in Bxb1 wt and Bxb1-based DNA modifying enzyme SH2 c121. FIGS. 27A-27D show results of a plasmid-based activity assay for the created DNA modifying enzymes with insertion of a heterologous DBD. Zif268 was fused between residues 285 and 286 or between the residues 467 and 468 of Bxb1 and between residues 285 and 286, residues 467 and 468, residues 478 and 479, and residues 489 and 490 of SH2cl21. (GGS)₄×(GGS)₄ linkers were used for the Bxb1-Zif268 (aa285) fusion (SEQ ID NO: 211), and (GGS)×(GGS)₈ were used for all the other fusions. The activities on the respective recombinase target sites (Batt-B_Batt-P52 (SEQ ID NO: 218), Batt-SH2-B (SEQ ID NO: 226), Batt-SH2-P (SEQ ID NO: 219)) and their 5-zif (B) (Batt-B-5-zif(B) (SEQ ID NO: 220), Batt-P52-5-zif(B) (SEQ ID NO: 221), Batt-SH2-B-5-zif(B) (SEQ ID NO: 222), Batt-SH2-P-5-zif(B) (SEQ ID NO: 223)) or 5-zif (A) (Batt-SH2-B-5-zif(A) (SEQ ID NO: 224), and Batt-SH2-P-5-zif(A) (SEQ ID NO: 225)) versions were tested. The upper band represents the unrecombined plasmid (line with two triangles), and the lower band represents the recombined plasmid (line with one triangle). M=Marker. Recombination efficiencies were calculated from ratios of recombined and non-recombined gel band intensities and indicated below the gel pictures.

### LIST OF SEQUENCES

The sequences referred to herein are disclosed in detail in the accompanying sequence listing. Exemplary sequences of the present invention are also listed in Table 1 below. Additional sequences are listed in the sequence listing.

TABLE 1
Exemplary sequences

| SEQ ID NO. | Name | Sequence |
|---|---|---|
| 1 | Zif268 target | GCGTGGGCG |
| | site | |
| 2 | ZNF217 | (T/A)(G/A)CAGAA(T/G/C) |
| | target site | |
| 3 | AvrXa7 TALE | TATAAACCCCCTCCAACCAGGTGCTAA |
| | target site | |
| 4 | Brec1 | MSILLTLHQSLSALLVDATSDEARKNLMDVLRDRQAFSERTWKVLLSVCRT |
| | | WAAWCKLNNRKWFPAEPEDVRDYLLHLQARGLAVNTILQHLAQLNMLHR |
| | | RFGLPRPGDSDAVSLVMRRIRRENVDAGERTKQALAFERTDFDQVRALME |
| | | NSERGQDIRTLALLGVAYNTLLRVSEIARIRIKDISRTDGGRMLIHISRTKTLVS |
| | | TAGVEKALSLGVTKLVERWISVSGVASDPNNYLFCQVRINGVAVPSATSRLS |
| | | TDVLRKIFEAAHRLIYGAKDGSGQRYLAWSGHSARVGAARDMARAGVSIAE |
| | | IMQAGGWTTVESVMNYIRNLDSETGAMVRLLEDGD |
| 5 | Brec1-Zif268 | MSILLTLHQSLSALLVDATSDEARKNLMDVLRDRQAFSERTWKVLLSVCRT |
| | (aa278) | WAAWCKLNNRKWFPAEPEDVRDYLLHLQARGLAVNTILQHLAQLNMLHR |
| | | RFGLPRPGDSDAVSLVMRRIRRENVDAGERTKQALAFERTDFDQVRALME |
| | | NSERGQDIRTLALLGVAYNTLLRVSEIARIRIKDISRTDGGRMLIHISRTKTLVS |
| | | TAGVEKALSLGVTKLVERWISVSGVASDPNNYLFCQVRINGVAVPSATSRLS |
| | | TDVLRKIFEAAHRLIYGAKDGTSAGGSGGSGGSGGSGGSGGSGGSGGSDRE |
| | | RPYACPVESCDRRFSRSDELTRHIRIHTGQKPFQCRICMRNFSRSDHLTTHIR |
| | | THTGEKPFACDICGRKFARSDERKRHTKIHLRQKDGRAGGSGGSGGSGGSG |
| | | GSGGSGGSGGSGPSGQRYLAWSGHSARVGAARDMARAGVSIAEIMQAG |
| | | GWTTVESVMNYIRNLDSETGAMVRLLEDGD |
| 6 | (GGS)₆-GRS- | GGSGGSGGSGGSGGSGGSGRSGGS |
| | GGS | |
| 7 | (GGS)₃-GRS- | GGSGGSGGSGRSGGSGRSGGSGGS |
| | GGS-GRS- | |
| | (GGS)₂ | |
| 8 | Vika | MTDLTPFPPLEHLEPDEFADLVRKAIKRDPQAGAHPAIQSAISHFQDEFVRR |
| | | QGEWQPATLQRLRNAWNVFVRWCTHQGIPALPARHQDVERYLIERRNEL |
| | | HRNTLKVHLWAIGKTHVISGLPNPCAHRYVKAQMAQITHQKVRERERIEQA |
| | | PAFRESDLDRLTELWSATRSVTQQRDLMIVSLAYETLLRKNNLEQMKVGDIE |
| | | FCQDGSALITIPFSKTNHSGRDDVRWISPQVANQVHAYLQLPNIDADPQCFL |
| | | LQRVKRSGKALNPESHNTLNGHHPVSEKLISRVFERAWRALNHETGPRYTG |
| | | HSARVGAAQDLLQEGYSTLQVMQAGGWSSEKMVLRYGRHLHAHTSAMA |
| | | QKRRQR |
| 9 | Vika-Zif268 | MTDLTPFPPLEHLEPDEFADLVRKAIKRDPQAGAHPAIQSAISHFQDEFVRR |
| | (aa172) | QGEWQPATLQRLRNAWNVFVRWCTHQGIPALPARHQDVERYLIERRNEL |
| | | HRNTLKVHLWAIGKTHVISGLPNPCAHRYVKAQMAQITHQKVRERERIEQA |
| | | PAFRESDLDRLTELWSATRSLEGGSGGSGGSGGSGGSGGSGGSGGSERPYA |
| | | CPVESCDRRFSRSDELTRHIRIHTGQKPFQCRICMRNFSRSDHLTTHIRTHTG |
| | | EKPFACDICGRKFARSDERKRHTKIHLRQKDGGSGGSGGSGGSGGSGGSGG |
| | | SGGSLYNVTQQRDLMIVSLAYETLLRKNNLEQMKVGDIEFCQDGSALITIPFS |
| | | KTNHSGRDDVRWISPQVANQVHAYLQLPNIDADPQCFLLQRVKRSGKALN |
| | | PESHNTLNGHHPVSEKLISRVFERAWRALNHETGPRYTGHSARVGAAQDLL |
| | | QEGYSTLQVMQAGGWSSEKMVLRYGRHLHAHTSAMAQKRRQR |
| 10 | Vika2-Zif268 | MTDLTPFPPLEHLEPDEFADLVRKAIKRDPQAGAHPAIQSAISHFQDEFVRR |
| | (aa172) | QGAWQPATLRRLRNAWNVFVRWCTLQGIPALPARHQDVERYLIERRNELH |
| | | RNTLKVHLWAIGKTHVISGLPNPCAHRYVKAQMAQITHQKVRERERIKQAP |
| | | AFRESDLDRLTELWSATRSLEGGSGGSGGSGGSGGSGGSGGSGGSERPYAC |
| | | PVESCDRRFSRSDELTRHIRIHTGQKPFQCRICMRNFSRSDHLTTHIRTHTGE |
| | | KPFACDICGRKFARSDERKRHTKIHLRQKDGGSGGSGGSGGSGGSGGSGGS |
| | | GGSLYNVTQQRDLMIVSLAYETLLRKNNLEQMRVGDIEFCQDGSALITIPFSK |
| | | TNHSGRDDVRWISPQVANQVRAYLQLPNIDADPQCFLLQRAGRSGRALNP |
| | | ESHNTLGGHHPVSEKLISRVFERAWRALNHGTGPRYTGHSARVGATLDLLQ |
| | | EGYSTLQVMQAGGWSSEKMVLRYGRHLHAHSSAMAQKRRQR |
| 11 | Vika3-Zif268 | MTDMTPFPPLEHLEPDEFADLVREAIKRDPQAGAHPAIQSAISHFQDEFVRR |
| | (aa172) | QGELQPATLQRLRYAWNVFVRWCTHRGIQALPARHQDVERYLIERRNELH |
| | | RKTLKVHLWAIGRTHVISGLPNPCAHRYVKAQMAQITHQKVRERERIKQAP |
| | | AFRESDLVRLTELWSATSSLEGGSGGSGGSGGSGGSGGSGGSGGSERPYAC |
| | | PVESCDRRFSRSDELTRHIRIHTGQKPFQCRICMRNFSRSDHLTTHIRTHTGE |
| | | KPFACDICGRKFARSDERKRHTKIHLRQKDGGSGGSGGSGGSGGSGGSGGS |
| | | GGSLYNVTQQRDLMIISLAYETLLRKSNLEQMKVGDIEFCQDGSALITIPFSKT |
| | | NHSGRDDVRWISPQVANLVRAYLQLPGVNADPQCFLLQRVRRSGKALNQE |
| | | GHNTLNGHHPVGEKLIGLVFERAWRALGHETGPRYTGHSARVGAAQDLLQ |
| | | EGYSTLQVMQAGGWSSEEMVLRYGRHLLAHNSAMAQKRRQR |
| 12 | Vika4-Zif268 | MTELTPFPPLEHLEPDEFADLVRKAIKRDPHAGAHPAIQRAISHFQDEFVRR |
| | (aa172) | QGELQPTTLRRLRYAWNDFVRWCTRQGILALPARHQDVERYLIERSSGLHR |
| | | NTLKANLWAIGKIHVISGLPNPCAHRHVKAQMAQIAHQKVRERERIRQAPA |
| | | FRESDLERLTELWSGTRSLEGGSGGSGGSGGSGGSGGSGGSGGSERPYACP |
| | | VESCDRRFSRSDELTRHIRIHTGQKPFQCRICMRNFSRSDHLTTHIRTHTGEK |
| | | PFACDICGRKFARSDERKRHTKIHLRQKDGGSGGSGGSGGSGGSGGSGGSG |
| | | GSLYNVIQQRDLMIISLAYETLLRKNNLEQMKVGDIEFCQDGSALITIPFSKTN |
| | | HSGRDDVRWISPRVASQVRAYLQLPNVDADPQCFLLQRVVRSGKALSPEG |
| | | HNTLDGHHPVSGMLISSVFERAWRALGHEAGPRYTGHSARVGAAQDLLQE |
| | | GYSILQVMQAGGWSSEEMVLRYGRHLHARNSAMAQKRRQR |
| 13 | Brec1- | MSILLTLHQSLSALLVDATSDEARKNLMDVLRDRQAFSERTWKVLLSVCRT |
| | ZFCCR5L | WAAWCKLNNRKWFPAEPEDVRDYLLHLQARGLAVNTILQHLAQLNMLHR |
| | (aa278) | RFGLPRPGDSDAVSLVMRRIRRENVDAGERTKQALAFERTDFDQVRALME |
| | | NSERGQDIRTLALLGVAYNTLLRVSEIARIRIKDISRTDGGRMLIHISRTKTLVS |
| | | TAGVEKALSLGVTKLVERWISVSGVASDPNNYLFCQVRINGVAVPSATSRLS |
| | | TDVLRKIFEAAHRLIYGAKDGLEGGSGGSGGSGGSGGSGGSGGSGGSERPF |
| | | QCRICMRNFSDRSNLSRHIRTHTGEKPFACDICGRKFAISSNLNSHTKIHTGS |
| | | QKPFQCRICMRNFSRSDNLARHIRTHTGEKPFACDICGRKFATSGNLTRHTKI |
| | | HLRGSGGSGGSGGSGGSGGSGGSGGSGGSLYSSGQRYLAWSGHSARVGA |
| | | ARDMARAGVSIAEIMQAGGWTTVESVMNYIRNLDSETGAMVRLLEDGD |
| 14 | Vika2 | MTDLTPFPPLEHLEPDEFADLVRKAIKRDPQAGAHPAIQSAISHFQDEFVRR |
| | | QGAWQPATLRRLRNAWNVFVRWCTLQGIPALPARHQDVERYLIERRNELH |
| | | RNTLKVHLWAIGKTHVISGLPNPCAHRYVKAQMAQITHQKVRERERIKQAP |
| | | AFRESDLDRLTELWSATRSVTQQRDLMIVSLAYETLLRKNNLEQMRVGDIEF |
| | | CQDGSALITIPFSKTNHSGRDDVRWISPQVANQVRAYLQLPNIDADPQCFLL |
| | | QRAGRSGRALNPESHNTLGGHHPVSEKLISRVFERAWRALNHGTGPRYTG |
| | | HSARVGATLDLLQEGYSTLQVMQAGGWSSEKMVLRYGRHLHAHSSAMAQ |
| | | KRRQR |
| 15 | Vika3 | MTDMTPFPPLEHLEPDEFADLVREAIKRDPQAGAHPAIQSAISHFQDEFVRR |
| | | QGELQPATLQRLRYAWNVFVRWCTHRGIQALPARHQDVERYLIERRNELH |
| | | RKTLKVHLWAIGRTHVISGLPNPCAHRYVKAQMAQITHQKVRERERIKQAP |
| | | AFRESDLVRLTELWSATSSVTQQRDLMIISLAYETLLRKSNLEQMKVGDIEFC |
| | | QDGSALITIPFSKTNHSGRDDVRWISPQVANLVRAYLQLPGVNADPQCFLL |
| | | QRVRRSGKALNQEGHNTLNGHHPVGEKLIGLVFERAWRALGHETGPRYTG |
| | | HSARVGAAQDLLQEGYSTLQVMQAGGWSSEEMVLRYGRHLLAHNSAMA |
| | | QKRRQR |
| 16 | Vika4 | MTELTPFPPLEHLEPDEFADLVRKAIKRDPHAGAHPAIQRAISHFQDEFVRR |
| | | QGELQPTTLRRLRYAWNDFVRWCTRQGILALPARHQDVERYLIERSSGLHR |
| | | NTLKANLWAIGKIHVISGLPNPCAHRHVKAQMAQIAHQKVRERERIRQAPA |
| | | FRESDLERLTELWSGTRSVIQQRDLMIISLAYETLLRKNNLEQMKVGDIEFCQ |
| | | DGSALITIPFSKTNHSGRDDVRWISPRVASQVRAYLQLPNVDADPQCFLLQR |
| | | VVRSGKALSPEGHNTLDGHHPVSGMLISSVFERAWRALGHEAGPRYTGHS |
| | | ARVGAAQDLLQEGYSILQVMQAGGWSSEEMVLRYGRHLHARNSAMAQK |
| | | RRQR |
| 17 | D7L | MSNLQTLHQNLSALLANATSDEARKNLMDVFRDRRAFSEATWKTLLSVCRT |
| | | WAAWCKLNNRKWFPAEPEDVRDYLLHLQVRGLAVNTIQRHLALLNMLHR |
| | | RSGLPRPGDSSAVSLVMRRIRKENVDAGERVRQALAFERTDFDKVRSLMGN |
| | | SDRCQDIRNLAFLGVAYNTLLRISEIARIRIKDISRTDGGRMLIHIGRTKTLVST |
| | | AGVEKALSLGVTRLVGRWISVSGVAGDPNNYLFCRVRKNGVAAPSATSQLS |
| | | TDVLRGVFAAAHRLVYGTKDDSGQGYLTWSGHSARVGAARDMARAGVSI |
| | | AEIMQAGGWTTVESVMSYLRNLDSETGAMVRLLEDGD |
| 18 | D7R | MSNIQTPHQSLSALLTDATSDVTRKNLADMFRDSQAFSEHTWKMLLSVCRS |
| | | WAAWCELNNRKWLPVEPEDVRDYLLHLQTRGLAVKTIQHHLGSLNMLHRR |
| | | AGLPRPGDSNAVSLVMRRIRRENVDAGERAQQALAFERTDFDQVRSLVENS |
| | | DRCQDIRNLAFLGVAYNTLLRISEIARIRVKDISRTDGGRMLIHIGRTKTLVSTA |
| | | GVEKALSLGVTKLVERWISVSGVADDPNNYLFCRVRRYGVAKPSATSQLSTY |
| | | ALQGIFGAAHRLVYGAKGDSGQKYLAWSGHSARVGAARDMARAGVPIPEI |
| | | MQAGGWTTVNSVMNYIRNLDSETGAMVRLLEDSD |
| 19 | D7L-Zif268 | MSNLQTLHQNLSALLANATSDEARKNLMDVFRDRRAFSEATWKTLLSVCRT |
| | (aa278) | WAAWCKLNNRKWFPAEPEDVRDYLLHLQVRGLAVNTIQRHLALLNMLHR |
| | | RSGLPRPGDSSAVSLVMRRIRKENVDAGERVRQALAFERTDFDKVRSLMGN |
| | | SDRCQDIRNLAFLGVAYNTLLRISEIARIRIKDISRTDGGRMLIHIGRTKTLVST |
| | | AGVEKALSLGVTRLVGRWISVSGVAGDPNNYLFCRVRKNGVAAPSATSQLS |
| | | TDVLRGVFAAAHRLVYGTKDDLEGGSGGSGGSGGSGGSGGSGGSGGSERP |
| | | YACPVESCDRRFSRSDELTRHIRIHTGQKPFQCRICMRNFSRSDHLTTHIRTH |
| | | TGEKPFACDICGRKFARSDERKRHTKIHLRQKDGGSGGSGGSGGSGGSGGS |
| | | GGSGGSLYSSGQGYLTWSGHSARVGAARDMARAGVSIAEIMQAGGWTTV |
| | | ESVMSYLRNLDSETGAMVRLLEDGD |
| 20 | D7R-Zif268 | MSNIQTPHQSLSALLTDATSDVTRKNLADMFRDSQAFSEHTWKMLLSVCRS |
| | (aa278) | WAAWCELNNRKWLPVEPEDVRDYLLHLQTRGLAVKTIQHHLGSLNMLHRR |
| | | AGLPRPGDSNAVSLVMRRIRRENVDAGERAQQALAFERTDFDQVRSLVENS |
| | | DRCQDIRNLAFLGVAYNTLLRISEIARIRVKDISRTDGGRMLIHIGRTKTLVSTA |
| | | GVEKALSLGVTKLVERWISVSGVADDPNNYLFCRVRRYGVAKPSATSQLSTY |
| | | ALQGIFGAAHRLVYGAKGDLEGGSGGSGGSGGSGGSGGSGGSGGSERPYA |
| | | CPVESCDRRFSRSDELTRHIRIHTGQKPFQCRICMRNFSRSDHLTTHIRTHTG |
| | | EKPFACDICGRKFARSDERKRHTKIHLRQKDGGSGGSGGSGGSGGSGGSGG |
| | | SGGSLYSSGQKYLAWSGHSARVGAARDMARAGVPIPEIMQAGGWTTVNS |
| | | VMNYIRNLDSETGAMVRLLEDSD |
| 21 | RecHTLV | MSKLLTLHQDLSALLADVTSDEARKNLMDMFRDRQAFPEHTWEMLLSVCR |
| | | SWAAWCESNNRKWFPAEPEDVRDYLLHLQARGLTVNTVQKHLAELNTLHR |
| | | RSGLPRPGDSNAVTLVMRRIRRENVDAGERAKQALAFERTDFDRVRSLMEN |
| | | SDRCLDIRNLAFLGVAYNTLLRISEIARIRVKDISRTDGGRMLIHIGRTKTLVSA |
| | | AGVEKALSLGVTKLVERWITASGVADDPNNYLFCRVRRYGVVVSSATSRLST |
| | | HAMQGIFGTAHRLIYGAKDDSGQRYLAWSGHSARVGAARDLARAGVSIAEI |
| | | MQAGGWTRVNSVMNYIRNLDSETGAMVRLLEDGD |
| 22 | RecHTLV- | MSKLLTLHQDLSALLADVTSDEARKNLMDMFRDRQAFPEHTWEMLLSVCR |
| | Zif268 | SWAAWCESNNRKWFPAEPEDVRDYLLHLQARGLTVNTVQKHLAELNTLHR |
| | (aa278) | RSGLPRPGDSNAVTLVMRRIRRENVDAGERAKQALAFERTDFDRVRSLMEN |
| | | SDRCLDIRNLAFLGVAYNTLLRISEIARIRVKDISRTDGGRMLIHIGRTKTLVSA |
| | | AGVEKALSLGVTKLVERWITASGVADDPNNYLFCRVRRYGVVVSSATSRLST |
| | | HAMQGIFGTAHRLIYGAKDDTSAGGSGGSGGSGGSGGSGGSGGSGGSDR |
| | | ERPYACPVESCDRRFSRSDELTRHIRIHTGQKPFQCRICMRNFSRSDHLTTHI |
| | | RTHTGEKPFACDICGRKFARSDERKRHTKIHLRQKDGRAGGSGGSGGSGGS |
| | | GGSGGSGGSGGSVNSGQRYLAWSGHSARVGAARDLARAGVSIAEIMQAG |
| | | GWTRVNSVMNYIRNLDSETGAMVRLLEDGD |
| 23 | RecFlex | MSKLQTIHQDLSALLVDVTSDEARRNLMDVLRDHQALSKHTWRVLLSVCRS |
| | | WAAWCELNNRKWFPAEPEDVRDYLLHLQTRGLTVNTIQQHLCQLNLLHRR |
| | | SGLPRPGDSNAVSLVMRRIRKENIDAGERVKQALAFERTDFDQVRSLMENS |
| | | DRCQDIRNLAFLGVAYNTLLRISEIARIRVRDITRTDGGRMLIHIGRTKTLVSA |
| | | AGEEKALSLGVTKLVERWISVSGVADDRNNYLFCRVKRNGVAAPSAFSQLST |
| | | PALHGVFAAAHRLIHGAKDASGQRYLTWSGHSARVGAARDMARAGVPVA |
| | | EIMQAGGWTTVESVMRYLRNLDSETGAMVRLLEDGD |
| 24 | loxFlex1 | CTCATTACATTTAACCAAAATTAAATGTAATGAG |
| 25 | loxFlex2 | TTATATTGTGATAACCAAAATTATCACAATATAA |
| 26 | loxFlex3 | CCATCTTTTGTTAGATTTGAATAACAAAAGATGG |
| 27 | loxFlex4 | TACACAGTGTATATTGATTTTTATACATTGTGTA |
| 28 | loxFlex5 | ATAACCTAATATAATTGTATTTATATTAGGTCAG |
| 29 | loxMECP2 | CACACTTTGTTTTATGTAGGCTATACCTTGATAA |
| 30 | RecFlex- | MSKLQTIHQDLSALLVDVTSDEARRNLMDVLRDHQALSKHTWRVLLSVCRS |
| | Zif268 | WAAWCELNNRKWFPAEPEDVRDYLLHLQTRGLTVNTIQQHLCQLNLLHRR |
| | (aa278) | SGLPRPGDSNAVSLVMRRIRKENIDAGERVKQALAFERTDFDQVRSLMENS |
| | | DRCQDIRNLAFLGVAYNTLLRISEIARIRVRDITRTDGGRMLIHIGRTKTLVSA |
| | | AGEEKALSLGVTKLVERWISVSGVADDRNNYLFCRVKRNGVAAPSAFSQLST |
| | | PALHGVFAAAHRLIHGAKDATSAGGSGGSGGSGGSGGSGGSGGSGGSDRE |
| | | RPYACPVESCDRRFSRSDELTRHIRIHTGQKPFQCRICMRNFSRSDHLTTHIR |
| | | THTGEKPFACDICGRKFARSDERKRHTKIHLRQKDGRAGGSGGSGGSGGSG |
| | | GSGGSGGSGGSGPSGQRYLTWSGHSARVGAARDMARAGVPVAEIMQAG |
| | | GWTTVESVMRYLRNLDSETGAMVRLLEDGD |
| 31 | ZFL1 | MERPFQCRICMRNFSFHSNLLAHIRTHTGQKPFQCRICMRNFSRKFVLDNHI |
| | | RTHTGEKPFACDICGRKFAQLGTLRRHTKIHLRGS |
| 32 | ZFL2 | MERPFQCRICMRNFSFHSNLLAHIRTHTGQKPFQCRICMRNFSRKFVLDNHI |
| | | RTHTGEKPFACDICGRKFAHASCLSRHTKIHLRGS |
| 33 | ZFR1 | MERPFQCRICMRNFSNQSGLCRHIRTHTGQKPFQCRICMRNFSFRSGLLQH |
| | | IRTHTGEKPFACDICGRKFAMRRYLRAHTKIHLRGS |
| 34 | ZFR2 | MERPFQCRICMRNFSTKRILTDHIRTHTGQKPFQCRICMRNFSFHSGLLAHI |
| | | RTHTGEKPFACDICGRKFAARRYLVQHTKIHLRGS |
| 35 | ZFR3 | MERPFQCRICMRNFSRKAHLTMHIRTHTGQKPFQCRICMRNFSAQSNLSR |
| | | HIRTHTGEKPFACDICGRKFAQLGNLRSHTKIHLRGS |
| 36 | ZFR4 | MERPYKCPECGKSFSRADNLTEHQRTHTGQKPFQCKTCQRKFSRSDHLKTH |
| | | TRTHTGEKPYRCKYCDRSFSISSNLQRHVRNIHTGQKPYKCDECGKNFTQSS |
| | | NLIVHKRIHLRGS |
| 37 | loxF8L | ATAAATCTGTGGAAACGCTGCTCCACAGATTTAT |
| 38 | loxF8R | CTAAGATTGTGTGAACGCTGCCACACAATCTTAG |
| 39 | loxF8Lflank | GGAGAATTCATTGCCAGCTATAAATCTGTGGAAACGCTGCTCCACAGATT |
| | | TATAGCTGGCAATGAATTCTCC |
| 40 | loxF8Rflank | TTTCTGCCAATCTTGTGTGCTAAGATTGTGTGAACGCTGCCACACAATCTT |
| | | AGCACACAAGATTGGCAGAAA |
| 41 | D7L-ZFL | MSNLQTLHQNLSALLANATSDEARKNLMDVFRDRRAFSEATWKTLLSVCRT |
| | (G10) | WAAWCKLNNRKWFPAEPEDVRDYLLHLQVRGLAVNTIQRHLALLNMLHR |
| | | RSGLPRPGDSSAVSLVMRRIRKENVDAGERVRQALAFERTDFDKVRSLMGN |
| | | SDRCQDIRNLAFLGVAYNTLLRISEIARIRIKDISRTDGGRMLIHIGRTKTLVST |
| | | AGVEKALSLGVTRLVGRWISVSGVAGDPNNYLFCRVRKNGVAAPSATSQLS |
| | | TDVLRGVFAAAHRLVYGTKDDTSAGGSGGSGGNGGSGGSGGSGGSGGSD |
| | | RERPFQCHICMRSFSFRSNLLAHIRTHTGQKPFQCHICMRNFSRKFVLDNHI |
| | | RTHTGEKPFACDICGRKFAQLGTLRRHAKIHLRGSGRADGSGGSGGSGGSG |
| | | RSGGSGRSGGSGPSGQGYLTWSGHSARVGAARDMARAGVSIAEIMQAGG |
| | | WTTVESVMSYLRNLDSETGAMVRLLEDGD |
| 42 | D7R-ZFR | MSNIQTPHQSLSALLTDATSDVTRKNLADMFRDSQAFSEHTWKMLLSVCRS |
| | (G10) | WAAWCELNNRKWLPVEPEDVRDYLLHLQTRGLAVKTIQHHLGSLNMLHRR |
| | | AGLPRPGDSNAVSLVMRRIRRENVDAGERAQQALAFERTDFDQVRSLVENS |
| | | DRCQDIRNLAFLGVAYNTLLRISEIARIRVKDISRTDGGRMLIHIGRTKTLVSTA |
| | | GVEKALSLGVTKLVERWISVSGVADDPNNYLFCRVRRYGVAKPSATSQLSTY |
| | | VLQGIFGAAHRLVYGAKGDTSAGGSGGSGGSGGSGGSGGSGGSGGSDRER |
| | | PYKCPESGKSFSRADNLTEHQRTQTGQKPFQCKICQRKFSRSEHLKTHTRAH |
| | | TGEKPYHCKHCDRGFSTSSNLQRHVRNIHTGLKPYKCDECGKNFAQSSNLIV |
| | | HKRTHLRGSGRTGGSGGSGGSGGSGRSGGSGRSGGSGPSGQKYLAWSGH |
| | | SARVGAARDMARAGVPIPEIMQAGGWTTVNSVMNYIRNLDSETGAMVRL |
| | | LEDSD |
| 43 | ZFL evolved | GGSGGSGGNGGSGGSGGSGGSGGSDRERPFQCHICMRSFSFRSNLLAHIRT |
| | (G10) | HTGQKPFQCHICMRNFSRFVLDNHIRTHTGEKPFACDICGRKFAQLGTLRRH |
| | | AKIHLRGSGRADGSGGSGGSGGSGRSGGSGRSGGS |
| 44 | ZFR evolved | GGSGGSGGSGGSGGSGGSGGSGGSDRERPYKCPESGKSFSRADNLTEHQR |
| | (G10) | TQTGQKPFQCKICQRKFSRSEHLKTHTRAHTGEKPYHCKHCDRGFSTSSNLQ |
| | | RHVRNIHTGLKPYKCDECGKNFAQSSNLIVHKRTHLRGSGRTGGSGGSGGS |
| | | GGSGRSGGSGRSGGS |
| 45 | HG2 | TTAAGATTGTGTTGTTTAATTTCCACAATTTTGT |
| 46 | HG2L | ATATATCTATAGATATAGATATCCACAGATATAT |
| 47 | TAL2295 | MNLTPDQVVAIASNIGGKQALETVQRLLPVLCQDHGLTPEQVVAIASHDGG |
| | | KQALETVQRLLPVLCQAHGLTPDQVVAIASNIGGKQALETVQRLLPVLCQAH |
| | | GLTPAQVVAIANNNGGKQALETVQRLLPVLCQDHGLTPDQVVAIASNIGGK |
| | | QALETVQRLLPVLCQDHGLTPEQVVAIASNIGGKQALETVQRLLPVLCQAHG |
| | | LTPDQVVAIANNNGGKQALETVQRLLPVLCQAHGLTPAQVVAIASHDGGK |
| | | QALETVQRLLPVLCQDHGLTPDQVVAIANNNGGKQALETVQRLLPVLCQD |
| | | HGLTPEQVVAIANNNGGKQALETVQRLLPVLCQAHGLTPDQVVAIANNNG |
| | | GKQALETVQRLLPVLCQAHGLTPAQVVAIASHDGGKQALETVQRLLPVLCQ |
| | | DHGLTPDQVVAIASNIGGKQALETVQRLLPVLCQDHGLTPEQVVAIASNIGG |
| | | KQALETVQRLLPVLCQAHGLTPDQVVAIASNIGGKQALETVQRLLPVLCQAH |
| | | GLTPAQVVAIANNNGGKQALETVQRLLPVLCQDHGL |
| 48 | TAL2295 | TACAGAAGCGGGCAAAGG |
| | target | |
| 49 | Zif268 | MERPYACPVESCDRRFSRSDELTRHIRIHTGQKPFQCRICMRNFSRSDHLTT |
| | | HIRTHTGEKPFACDICGRKFARSDERKRHTKIH |
| 50 | Zif268 target | GCGTGGGCG |
| 51 | Brec1- | MSILLTLHQSLSALLVDATSDEARKNLMDVLRDRQAFSERTWKVLLSVCRT |
| | TAL2295 | WAAWCKLNNRKWFPAEPEDVRDYLLHLQARGLAVNTILQHLAQLNMLHR |
| | (aa278) | RFGLPRPGDSDAVSLVMRRIRRENVDAGERTKQALAFERTDFDQVRALME |
| | | NSERGQDIRTLALLGVAYNTLLRVSEIARIRIKDISRTDGGRMLIHISRTKTLVS |
| | | TAGVEKALSLGVTKLVERWISVSGVASDPNNYLFCQVRINGVAVPSATSRLS |
| | | TDVLRKIFEAAHRLIYGAKDGTSAGGSGGSGGSGGSGGSGGSGGSGGSDR |
| | | MVDLRTLGYSQQQQEKIKPKVRSTVAQHHEALVGHGFTHAHIVALSQHPA |
| | | ALGTVAVKYQDMIAALPEATHEAIVGVGKQWSGARALEALLTVAGELRGPP |
| | | LQLDTGQLLKIAKRGGVTAVEAVHAWRNALTGAPLNLTPDQVVAIASNIGG |
| | | KQALETVQRLLPVLCQDHGLTPEQVVAIASHDGGKQALETVQRLLPVLCQA |
| | | HGLTPDQVVAIASNIGGKQALETVQRLLPVLCQAHGLTPAQVVAIANNNGG |
| | | KQALETVQRLLPVLCQDHGLTPDQVVAIASNIGGKQALETVQRLLPVLCQDH |
| | | GLTPEQVVAIASNIGGKQALETVQRLLPVLCQAHGLTPDQVVAIANNNGGK |
| | | QALETVQRLLPVLCQAHGLTPAQVVAIASHDGGKQALETVQRLLPVLCQDH |
| | | GLTPDQVVAIANNNGGKQALETVQRLLPVLCQDHGLTPEQVVAIANNNGG |
| | | KQALETVQRLLPVLCQAHGLTPDQVVAIANNNGGKQALETVQRLLPVLCQA |
| | | HGLTPAQVVAIASHDGGKQALETVQRLLPVLCQDHGLTPDQVVAIASNIGG |
| | | KQALETVQRLLPVLCQDHGLTPEQVVAIASNIGGKQALETVQRLLPVLCQAH |
| | | GLTPDQVVAIASNIGGKQALETVQRLLPVLCQAHGLTPAQVVAIANNNGGK |
| | | QALETVQRLLPVLCQDHGLTPEQVVAIANNNGGRPALESIVAQLSRPDPALA |
| | | ALTNDHLVALACLGGRPALDAVKKGLPHAPALIKRTNRRIPERTSHRVAGRA |
| | | GGSGGSGGSGGSGGSGGSGGSGGSGPSGQRYLAWSGHSARVGAARDMA |
| | | RAGVSIAEIMQAGGWTTVESVMNYIRNLDSETGAMVRLLEDGD |
| 52 | loxBTR-5- | TACAGAAGCGGGCAAAGGtactcAACCCACTGCTTAAGCCTCAATAAAGCT |
| | TAL2295 (A) | TGCCTTatagaCCTTTGCCCGCTTCTGTA |
| 53 | loxBTR-5- | CCTTTGCCCGCTTCTGTAtactcAACCCACTGCTTAAGCCTCAATAAAGCTT |
| | TAL2295 (B) | GCCTTatagaTACAGAAGCGGGCAAAGG |
| 54 | ZFL1 target f | GGGAGAATTCATTGCCAGCT |
| 55 | ZFR1 target f | CACACAAGATTGGCAGAAAA |
| 56 | ZFL1 target r | AGCTGGCAATGAATTCTCCC |
| 57 | ZFR1 target r | TTTTCTGCCAATCTTGTGTG |
| 58 | ZFL1 + linker | GGSGGSGGSGGSGGSGGSGGSGGSDRERPFQCRICMRNFSFHSNLLAHIRT |
| | | HTGQKPFQCRICMRNFSRKFVLDNHIRTHTGEKPFACDICGRKFAQLGTLRR |
| | | HTKIHLRGSGRAGGSGGSGGSGGSGGSGGSGGSGGS |
| 59 | ZFR4 + linker | GGSGGSGGSGGSGGSGGSGGSGGSDRERPYKCPECGKSFSRADNLTEHQR |
| | | THTGQKPFQCKTCQRKFSRSDHLKTHTRTHTGEKPYRCKYCDRSFSISSNLQR |
| | | HVRNIHTGQKPYKCDECGKNFTQSSNLIVHKRIHLRGSGRAGGSGGSGGSG |
| | | GSGGSGGSGGSGGS |
| 60 | ZFCCR5L | MDYKDHDGDYKDHDIDYKDDDDKMAPKKKRKVGIHGVPAAMAERPFQC |
| | | RICMRNFSDRSNLSRHIRTHTGEKPFACDICGRKFAISSNLNSHTKIHTGSQKP |
| | | FQCRICMRNFSRSDNLARHIRTHTGEKPFACDICGRKFATSGNLTRHTKIHLR |
| | | GS |
| 61 | ZFCCR5L | GATGAGGATGAC |
| | target | |
| 62 | loxBTR-5- | GATGAGGATGACtactcAACCCACTGCTTAAGCCTCAATAAAGCTTGCCTT |
| | ZFCCR5L (A) | atagaGTCATCCTCATC |
| 63 | loxBTR-5- | GTCATCCTCATCtactcAACCCACTGCTTAAGCCTCAATAAAGCTTGCCTTat |
| | ZFCCR5L (B) | agaGATGAGGATGAC |
| 64 | Brec1-Zif268 | MSILLTLHQSLSALLVDATSDEARKNLMDVLRDRQAFSERTWKVLLSVCRT |
| | (aa278)*Rmut | WAAWCKLNNRKWFPAEPEDVRDYLLHLQARGLAVNTILQHLAQLNMLHR |
| | | RFGLPRPGDSDAVSLVMRRIRRENVDAGERTKQALAFERTDFDQVRALME |
| | | NSERGQDIRTLALLGVAYNTLLRVSEIARIRIKDISRTDGGRMLIHISRTKTLVS |
| | | TAGVEKALSLGVTKLVERWISVSGVASDPNNYLFCQVRINGVAVPSATSRLS |
| | | TDVLRKIFEAAHRLIYGAKDGTSAGGSGGSGGSGGSGGSGGSGGSGGSDRE |
| | | RPYACPVESCDRRFSRSDELTRHIRIHTGQKPFQCRICMRNFSRSDHLTTHIR |
| | | THTGEKPFACDICGRKFARSDERKRHTKIHLRQKDGRAGGSGGSGGSGGSG |
| | | GSGGSGRSGGSGPSGQRYLAWSGHSARVGAARDMARAGVSIAEIMQAGG |
| | | WTTVESVMNYIRNLDSETGAMVRLLEDGD |
| 65 | loxBTR | AACCCACTGCTTAAGCCTCAATAAAGCTTGCCTT |
| 66 | loxBTR-5-zif | AGCGTGGGCGTACTGAACCCACTGCTTAAGCCTCAATAAAGCTTGCCTTC |
| | (A) | AGTACGCCCACGCT |
| 67 | loxBTR-5-zif | ACGCCCACGCtactcAACCCACTGCTTAAGCCTCAATAAAGCTTGCCTTatag |
| | (B) | aGCGTGGGCGT |
| 68 | VOX | AATAGGTCTGAGAACGCCCATTCTCAGACGTATT |
| 69 | vox-5-zif (A) | tGCGTGGGCGtgtctAATAGGTCTGAGAACGCCCATTCTCAGACGTATTtac |
| | | taCGCCCACGCa |
| 70 | vox-5-zif (B) | aCGCCCACGCagtctAATAGGTCTGAGAACGCCCATTCTCAGACGTATTtact |
| | | cGCGTGGGCGt |
| 71 | vox2 | GTTAGGTCTGAGAATCTGTATTCTCTCTCTTGAA |
| 72 | vox2-5-zif (A) | tGCGTGGGCGtgtctGTTAGGTCTGAGAATCTGTATTCTCTCTCTTGAAtact |
| | | aCGCCCACGCa |
| 73 | vox2-5-zif (B) | aCGCCCACGCagtctGTTAGGTCTGAGAATCTGTATTCTCTCTCTTGAAtact |
| | | cGCGTGGGCGt |
| 74 | vox4 | AAGACCTTAGTGATGCCCAGTTGACCCAGGACGC |
| 75 | vox4-5-zif (A) | tGCGTGGGCGtgtctAAGACCTTAGTGATGCCCAGTTGACCCAGGACGCtac |
| | | taCGCCCACGCa |
| 76 | vox4-5-zif (B) | aCGCCCACGCtactcAAGACCTTAGTGATGCCCAGTTGACCCAGGACGCata |
| | | gaGCGTGGGCGt |
| 77 | HGZF1 | aagaaatgtaggaaacccACAAATCTGTGGAAAGTAAACGACACACTCCTAAa |
| | | aaacAATGGGTGGAAGa |
| 78 | HGZF2 | atacagaatggaaattgaATAAATGCGTGGTGAATGGACCACAGTCTCTCAGca |
| | | cagAAGCAAGAGCAGg |
| 79 | HGZF3 | tagcaatctatcgattagATATTTGTGTGGAAGAAAGCACAGAAAATCTGATag |
| | | atttTAGCTAAGGAAG |
| 80 | HGZF4 | atagtactagcacctactATAAATGGGAGGATATATAGGTATACAATGTTAAat |
| | | aaAAGATATAGAAGta |
| 81 | HGZF5 | tatatatgcattctatctGAAAAATTGTGTGCTTAGATATACAAAGGTTTACccca |
| | | taATAATGAATgtt |
| 82 | HGZF6 | gaaATTAATTGGatcctaATAAAACTGGTAATGGATTTGCACCCAATCATAT |
| | | ctcacatctctaacaggg |
| 83 | HGZF7 | ccctgttagagatgtgagATATGATTGGGTGCAAATCCATTACCAGTTTTATtag |
| | | gatCCAATTAATttc |
| 84 | HGZF8 | aataataaaataaagaagCTAATATTATATAGAGCTAGAACCAGAGATATATtt |
| | | tgtaATAATGCATtgt |
| 85 | loxFlex1-5-zif | ACGCCCACGCtactcCTCATTACATTTAACCAAAATTAAATGTAATGAGatag |
| | (B) | aGCGTGGGCGT |
| 86 | loxFlex2-5-zif | ACGCCCACGCtactcTTATATTGTGATAACCAAAATTATCACAATATAAatag |
| | (B) | aGCGTGGGCGT |
| 87 | loxFlex3-5-zif | ACGCCCACGCtactgCCATCTTTTGTTAGATTTGAATAACAAAAGATGGata |
| | (B) | gaGCGTGGGCGT |
| 88 | loxFlex4-5-zif | ACGCCCACGCtactcTACACAGTGTATATTGATTTTTATACATTGTGTAatag |
| | (B) | aGCGTGGGCGT |
| 89 | loxFlex5-5-zif | ACGCCCACGCtactgATAACCTAATATAATTGTATTTATATTAGGTCAGatag |
| | (B) | aGCGTGGGCGT |
| 90 | loxMECP2-5- | ACGCCCACGCtactgCACACTTTGTTTTATGTAGGCTATACCTTGATAAatag |
| | zif (B) | aGCGTGGGCGT |
| 91 | D7L-ZFL(G10) | MSNLQTLHQNLSALLANATSDEARKNLMDVFRDRRAFSEATWKTLLSVCRT |
| | | WAAWCKLNNRKWFPAEPEDVRDYLLHLQVRGLAVNTIQRHLALLNMLHR |
| | | RSGLPRPGDSSAVSLVMRRIRKENVDAGERVRQALAFERTDFDKVRSLMGN |
| | | SDRCQDIRNLAFLGVAYNTLLRISEIARIRIKDISRTDGGRMLIHIGRTKTLVST |
| | | AGVEKALSLGVTRLVGRWISVSGVAGDPNNYLFCRVRKNGVAAPSATSQLS |
| | | TDVLRGVFAAAHRLVYGTKDDTSAGGSGGSGGSGGSGGSGGSGGSGGSD |
| | | RERPFQCRICMRNFSFHSNLLAHIRTHTGQKPFQCRICMRNFSRKFVLDNHI |
| | | RTHTGEKPFACDICGRKFAQLGTLRRHTKIHLRGSGRAGGSGGSGGSGGSG |
| | | GSGGSGGSGGSGPSGQGYLTWSGHSARVGAARDMARAGVSIAEIMQAG |
| | | GWTTVESVMSYLRNLDSETGAMVRLLEDGD |
| 92 | D7R- | MSNIQTPHQSLSALLTDATSDVTRKNLADMFRDSQAFSEHTWKMLLSVCRS |
| | ZFR(G10) | WAAWCELNNRKWLPVEPEDVRDYLLHLQTRGLAVKTIQHHLGSLNMLHRR |
| | | AGLPRPGDSNAVSLVMRRIRRENVDAGERAQQALAFERTDFDQVRSLVENS |
| | | DRCQDIRNLAFLGVAYNTLLRISEIARIRVKDISRTDGGRMLIHIGRTKTLVSTA |
| | | GVEKALSLGVTKLVERWISVSGVADDPNNYLFCRVRRYGVAKPSATSQLSTY |
| | | ALQGIFGAAHRLVYGAKGDTSAGGSGGSGGSGGSGGSGGSGGSGGSDRER |
| | | PYKCPECGKSFSRADNLTEHQRTHTGQKPFQCKTCQRKFSRSDHLKTHTRTH |
| | | TGEKPYRCKYCDRSFSISSNLQRHVRNIHTGQKPYKCDECGKNFTQSSNLIVH |
| | | KRIHLRGSGRAGGSGGSGGSGGSGGSGGSGGSGGSGPSGQKYLAWSGHS |
| | | ARVGAARDMARAGVPIPEIMQAGGWTTVNSVMNYIRNLDSETGAMVRLL |
| | | EDSD |
| 201 | Cre | MSNLLTVHQNLPALPVDATSDEVRKNLMDMFRDRQAFSEHTWKMLLSVC |
| | | RSWAAWCKLNNRKWFPAEPEDVRDYLLYLQARGLAVKTIQQHLGQLNML |
| | | HRRSGLPRPSDSNAVSLVMRRIRKENVDAGERAKQALAFERTDFDQVRSLM |
| | | ENSDRCQDIRNLAFLGIAYNTLLRIAEIARIRVKDISRTDGGRMLIHIGRTKTLV |
| | | STAGVEKALSLGVTKLVERWISVSGVADDPNNYLFCRVRKNGVAAPSATSQL |
| | | STRALEGIFEATHRLIYGAKDDSGQRYLAWSGHSARVGAARDMARAGVSIP |
| | | EIMQAGGWTNVNIVMNYIRNLDSETGAMVRLLEDGD |
| 204 | A118 | MKAAIYIRVSTQEQVENYSIQAQTEKLTALCRSKDWDVYDIFIDGGYSGSNM |
| | | NRPALNEMLSKLHEIDAVVVYRLDRLSRSQRDTITLIEEYFLKNNVEFVSLSETL |
| | | DTSSPFGRAMIGILSVFAQLERETIRDRMVMGKIKRIEAGLPLTTAKGRTFGY |
| | | DVIDTKLYINEEEAKQLQLIYDIFEEEQSITFLQKRLKKLGFKVRTYNRYNNWLT |
| | | NDLYCGYVSYKDKVHVKGIHEPIISEEQFYRVQEIFTRMGKNPNMNRDSASL |
| | | LNNLVVCSKCGLGFVHRRKDTMSRGKKYHYRYYSCKTYKHTHELEKCGNKI |
| | | WRADKLEELIINRVNNYSFASRNVDKEDELDSLNEKLKIEHAKKKRLFDLYING |
| | | SYEVSELDSMMNDIDAQINYYESQIEANEELKKNKKIQENLADLATVDFDSLE |
| | | FREKQLYLKSLINKIYIDGEQVTIEWL |
| 205 | Bxb1 | MRALVVIRLSRVTDATTSPERQLESCQQLCAQRGWDVVGVAEDLDVSGAV |
| | | DPFDRKRRPNLARWLAFEEQPFDVIVAYRVDRLTRSIRHLQQLVHWAEDHK |
| | | KLVVSATEAHFDTTTPFAAVVIALMGTVAQMELEAIKERNRSAAHFNIRAGK |
| | | YRGSLPPWGYLPTRVDGEWRLVPDPVQRERILEVYHRVVDNHEPLHLVAH |
| | | DLNRRGVLSPKDYFAQLQGREPQGREWSATALKRSMISEAMLGYATLNGK |
| | | TVRDDDGAPLVRAEPILTREQLEALRAELVKTSRAKPAVSTPSLLLRVLFCAVC |
| | | GEPAYKFAGGGRKHPRYRCRSMGFPKHCGNGTVAMAEWDAFCEEQVLDL |
| | | LGDAERLEKVWVAGSDSAVELAEVNAELVDLTSLIGSPAYRAGSPQREALDA |
| | | RIAALAARQEELEGLEARPSGWEWRETGQRFGDWWREQDTAAKNTWLRS |
| | | MNVRLTFDVRGGLTRTIDFGDLQEYEQHLRLGSVVERLHTGMS |
| 206 | SH1_cl29 | MRALVVIRLSRVTDATTSPERQLESCQQLCAQRGWDVVGVAEDLDVSGAV |
| | | DPFDRKRRPNLARWLAFEEQPFDVIVAYRVDRLTRSVRYLQQLVHWAEDHK |
| | | KLVVSATEAHFDTTTPFAAVVIALMGTVAQMELEAIRERSRSAAHFNIRAGK |
| | | YRGSLPPWGYLPTRVDGEWRLVPDPVQRERILEVYHRVVDNHESLHRVAH |
| | | DLNRRGVLSPKDYFAQLRGREPQGRKWSATALKCSLTSEAMLGYATLNGKT |
| | | VRNDDGAPLVRAEPILTREQLEALRAELVKTSRAKPAVSTPSLLLRVLFCAVCG |
| | | EPAYKFTGGGRKHPRYRCRSMGFPEHCGNGTVVMAEWDAFCEEQVLGLLE |
| | | DAERLEKVWVAGSDSAVELAEVNAELVDLTSLIGSPAYRAGSPQREALDARI |
| | | AALAARQGELEGLEARPSGWEWRATGQRFGDWWREQGTAAKNAWLRS |
| | | MNVRLTFDVRGGLTRTIDFGDLREYEQHLRSGSVVDRLHTRMP |
| 207 | SH2_cl21 | MRALVVIRLSRVTDATTSPERQLESCQQLCTQRGWDVVGVAEDLDVSGAV |
| | | DPFDCKRRPNLARWLAFEEQPFDVIVAYRVDRLTRSVRHLQQLVHWAEDH |
| | | KKLVVSATEAHFDTTTPFAAVVIALMGTVAQMELEAIKERSRSAAHFNIRAG |
| | | KYRGSLPPWGYLPTRVDGEWRLAPDPVQRERILEVYHRVVDNHEPLHLVAH |
| | | DLNRRGVLSPKDYFAQLRGREPQGRKWSATALKRSMISEAMLGYATLNGKT |
| | | VRGDDGAPLVRAEPILTREQLEALRAELVKTSRAKPAVSTPSLLLRVLFCAVCG |
| | | EPAYKFAGGGRKHPRYRCRSMGFPKHCGNGTVAMAEWDAFCEEQVLDLL |
| | | GDAERLEKVWVAGSDSAVELAEVNAELVDLTSLIGSPAYRAGSPQREALDAR |
| | | IAALAARQEELEGLEARPSGWEWRGTGQRFGDWWREQDTAAKNTWLRS |
| | | MNVRLTFDVRGGLTRTIDLGDLQEYEQHLRLGSVVERSHTGMP |
| 208 | SH2_cl29 | MRALVVIRLSRVTDATTSPERQLESCQQLCAQRGWDVVGVAEDLDVSGAV |
| | | DPFDRKRRPNLARWLAFEEQPFDVIVAYRVDRLTRSVRHLQQLVHWAEDH |
| | | KKLVVSATEAHFDTTTPFAAVVIALMGTVAQMELEAIRERSRSATHFNIRAG |
| | | KYRGSLPPWGYLPTRVDGEWRLVPDPVQRERILEVYHRVVDNHEPLHLVAH |
| | | DLNRRGVLSPKDYFAQLRGRKPQGRKWSATALKRSMISEAMLGYATLNGKT |
| | | VRDDDGAPLVRAEPILTREQQEALRVELVKTSRAKPAVSAPSLLLRVLFCAVC |
| | | GEPAYKFAGGGRKHPRYRCRSMGFPKHCGNGTVAMAEWDAFCEEQVLNL |
| | | LGDAERLEKVWVAGSDSAVELAEVNAELVDLTSLIGSPAYRAGSPQREALDT |
| | | RIAALAARQEELEGLEARPSGWEWRGTGQRFGDWWREQDTAAKNTWLR |
| | | SMNVRLAFDVRGGLTRTIDVGDLREYGQHLRLGSVVERLRTGMS |
| 209 | SH3_cl326 | MRALVVIRLSRVTDATTSPERQLESCQQLCAQRGWDVVGVAEDLDVSGAV |
| | | DPFDRMRRPSLARWLAFEEQPFDVIVAYRVDRLTRSVRHLQQLVHWAEDH |
| | | KKLVVSATEAHFDTTTPFAAVVIALMGTVAQMELEAIKERIRSAVRFNIRAGK |
| | | YRGSLPPWGYLPTRVDGEWRLVPDPAQRERILEVYHRVVDNHEPLHLVAH |
| | | DLNRRGVLSPKDYFAQLQGREPQGRKWSATALRRSMISGAMLGYATLNGK |
| | | TVRGDDGAPLVRAEPILTREQQEALRAELAKTSRAKPAVSSPSLLLRVLFCAV |
| | | CGEPAYKFVGGGRKHPRYRCRSMGSPKHCGNGTVVVAEWDAFCEEQVLD |
| | | LLGDVERLEKVWVAGSDFAVELAEVNAELVDLTSLIGSPAYRAGSPQREALD |
| | | ARIAALAARQEELEGLEARPSGWEWRETGQRFGDWWREQDTAAKNTWL |
| | | RSMNVRLTFDARGGLTRTIDFGDLQEYEQHLRLGGVVERLHTGMS |
| 210 | SH4_cl779 | MRALVVIRLSRVTDATTSPERQLESCQQLCAQRGWDVVGVAEDLDVSGAV |
| | | DPFDRKRRPNLARWLAFEEQPFDVIVAYRVDRLTRSVRHLQQLVHWAEDH |
| | | KKLVVSATEAHFDTTTPFAAVVIALMGTVAQMELEAIKERNRSAAHFNIRAG |
| | | KYRGSLPPWGYLPTRVDGEWRLAPDPVQRERILEVYHRVVNNHEPLHLVAH |
| | | DLNRRGVLSPKDYFAQLQGREPQGRKWSATALKRSMISEAMLGYATLNGK |
| | | TVRDDGGAPLVRAEPILTRGQLEALRAELAKTSRAKPAVSTPSLLLRVLFCAVC |
| | | GEPAYKFAGGGRKHPRYRCRSMGFPKHCGNGTVVMAEWDAFCEEQVLDL |
| | | LGDAERLEKVWITGSDSAVELAEVSAELVDLTSLIGSPAYRAGSPQREALDAR |
| | | IAALAARQEELEGLEVRPSGWEWRETGQRFGDWWREQDTAAKNTWLRS |
| | | MNVRLTFDVRGGLTRTIDFGDLQEYEQHLRLGSVVERLHTGMS |
| 211 | Bxb1-Zif268 | MRALVVIRLSRVTDATTSPERQLESCQQLCAQRGWDVVGVAEDLDVSGAV |
| | (aa285) | DPFDRKRRPNLARWLAFEEQPFDVIVAYRVDRLTRSIRHLQQLVHWAEDHK |
| | | KLVVSATEAHFDTTTPFAAVVIALMGTVAQMELEAIKERNRSAAHFNIRAGK |
| | | YRGSLPPWGYLPTRVDGEWRLVPDPVQRERILEVYHRVVDNHEPLHLVAH |
| | | DLNRRGVLSPKDYFAQLQGREPQGREWSATALKRSMISEAMLGYATLNGK |
| | | TVRDDDGAPLVRAEPILTREQLEALRAELVKTGGSGGSGGSGGSERPYACPV |
| | | ESCDRRFSRSDELTRHIRIHTGQKPFQCRICMRNFSRSDHLTTHIRTHTGEKP |
| | | FACDICGRKFARSDERKRHTKIHLRQKDGGSGGSGGSGGSSRAKPAVSTPSL |
| | | LLRVLFCAVCGEPAYKFAGGGRKHPRYRCRSMGFPKHCGNGTVAMAEWD |
| | | AFCEEQVLDLLGDAERLEKVWVAGSDSAVELAEVNAELVDLTSLIGSPAYRA |
| | | GSPQREALDARIAALAARQEELEGLEARPSGWEWRETGQRFGDWWREQD |
| | | TAAKNTWLRSMNVRLTFDVRGGLTRTIDFGDLQEYEQHLRLGSVVERLHTG |
| | | MS |
| 212 | Bxb1-Zif268 | MRALVVIRLSRVTDATTSPERQLESCQQLCAQRGWDVVGVAEDLDVSGAV |
| | (aa467) | DPFDRKRRPNLARWLAFEEQPFDVIVAYRVDRLTRSIRHLQQLVHWAEDHK |
| | | KLVVSATEAHFDTTTPFAAVVIALMGTVAQMELEAIKERNRSAAHFNIRAGK |
| | | YRGSLPPWGYLPTRVDGEWRLVPDPVQRERILEVYHRVVDNHEPLHLVAH |
| | | DLNRRGVLSPKDYFAQLQGREPQGREWSATALKRSMISEAMLGYATLNGK |
| | | TVRDDDGAPLVRAEPILTREQLEALRAELVKTSRAKPAVSTPSLLLRVLFCAVC |
| | | GEPAYKFAGGGRKHPRYRCRSMGFPKHCGNGTVAMAEWDAFCEEQVLDL |
| | | LGDAERLEKVWVAGSDSAVELAEVNAELVDLTSLIGSPAYRAGSPQREALDA |
| | | RIAALAARQEELEGLEARPSGWEWRETGQRFGDWWREQDTAAKNTWLRS |
| | | MNVRLTFDVRGGSGGSGGSGGSGGSGGSGGSGGSERPYACPVESCDRRFS |
| | | RSDELTRHIRIHTGQKPFQCRICMRNFSRSDHLTTHIRTHTGEKPFACDICGR |
| | | KFARSDERKRHTKIHLRQKDGGSGGSGGSGGSGGSGGSGGSGGSGGLTRTI |
| | | DFGDLQEYEQHLRLGSVVERLHTGMS |
| 213 | SH2 cl21- | MRALVVIRLSRVTDATTSPERQLESCQQLCTQRGWDVVGVAEDLDVSGAV |
| | Zif268 | DPFDCKRRPNLARWLAFEEQPFDVIVAYRVDRLTRSVRHLQQLVHWAEDH |
| | (aa285) | KKLVVSATEAHFDTTTPFAAVVIALMGTVAQMELEAIKERSRSAAHFNIRAG |
| | | KYRGSLPPWGYLPTRVDGEWRLAPDPVQRERILEVYHRVVDNHEPLHLVAH |
| | | DLNRRGVLSPKDYFAQLRGREPQGRKWSATALKRSMISEAMLGYATLNGKT |
| | | VRGDDGAPLVRAEPILTREQLEALRAELVKTGGSGGSGGSGGSGGSGGSGG |
| | | SGGSERPYACPVESCDRRFSRSDELTRHIRIHTGQKPFQCRICMRNFSRSDHL |
| | | TTHIRTHTGEKPFACDICGRKFARSDERKRHTKIHLRQKDGGSGGSGGSGGS |
| | | GGSGGSGGSGGSSRAKPAVSTPSLLLRVLFCAVCGEPAYKFAGGGRKHPRYR |
| | | CRSMGFPKHCGNGTVAMAEWDAFCEEQVLDLLGDAERLEKVWVAGSDSA |
| | | VELAEVNAELVDLTSLIGSPAYRAGSPQREALDARIAALAARQEELEGLEARP |
| | | SGWEWRGTGQRFGDWWREQDTAAKNTWLRSMNVRLTFDVRGGLTRTI |
| | | DLGDLQEYEQHLRLGSVVERSHTGMP |
| 214 | SH2 cl21- | MRALVVIRLSRVTDATTSPERQLESCQQLCTQRGWDVVGVAEDLDVSGAV |
| | Zif268 | DPFDCKRRPNLARWLAFEEQPFDVIVAYRVDRLTRSVRHLQQLVHWAEDH |
| | (aa467) | KKLVVSATEAHFDTTTPFAAVVIALMGTVAQMELEAIKERSRSAAHFNIRAG |
| | | KYRGSLPPWGYLPTRVDGEWRLAPDPVQRERILEVYHRVVDNHEPLHLVAH |
| | | DLNRRGVLSPKDYFAQLRGREPQGRKWSATALKRSMISEAMLGYATLNGKT |
| | | VRGDDGAPLVRAEPILTREQLEALRAELVKTSRAKPAVSTPSLLLRVLFCAVCG |
| | | EPAYKFAGGGRKHPRYRCRSMGFPKHCGNGTVAMAEWDAFCEEQVLDLL |
| | | GDAERLEKVWVAGSDSAVELAEVNAELVDLTSLIGSPAYRAGSPQREALDAR |
| | | IAALAARQEELEGLEARPSGWEWRGTGQRFGDWWREQDTAAKNTWLRS |
| | | MNVRLTFDVRGGSGGSGGSGGSGGSGGSGGSGGSERPYACPVESCDRRFS |
| | | RSDELTRHIRIHTGQKPFQCRICMRNFSRSDHLTTHIRTHTGEKPFACDICGR |
| | | KFARSDERKRHTKIHLRQKDGGSGGSGGSGGSGGSGGSGGSGGSGGLTRTI |
| | | DLGDLQEYEQHLRLGSVVERSHTGMP |
| 215 | SH2 cl21- | MRALVVIRLSRVTDATTSPERQLESCQQLCTQRGWDVVGVAEDLDVSGAV |
| | Zif268 | DPFDCKRRPNLARWLAFEEQPFDVIVAYRVDRLTRSVRHLQQLVHWAEDH |
| | (aa478) | KKLVVSATEAHFDTTTPFAAVVIALMGTVAQMELEAIKERSRSAAHFNIRAG |
| | | KYRGSLPPWGYLPTRVDGEWRLAPDPVQRERILEVYHRVVDNHEPLHLVAH |
| | | DLNRRGVLSPKDYFAQLRGREPQGRKWSATALKRSMISEAMLGYATLNGKT |
| | | VRGDDGAPLVRAEPILTREQLEALRAELVKTSRAKPAVSTPSLLLRVLFCAVCG |
| | | EPAYKFAGGGRKHPRYRCRSMGFPKHCGNGTVAMAEWDAFCEEQVLDLL |
| | | GDAERLEKVWVAGSDSAVELAEVNAELVDLTSLIGSPAYRAGSPQREALDAR |
| | | IAALAARQEELEGLEARPSGWEWRGTGQRFGDWWREQDTAAKNTWLRS |
| | | MNVRLTFDVRGGLTRTIDLGDGGSGGSGGSGGSGGSGGSGGSGGSERPYA |
| | | CPVESCDRRFSRSDELTRHIRIHTGQKPFQCRICMRNFSRSDHLTTHIRTHTG |
| | | EKPFACDICGRKFARSDERKRHTKIHLRQKDGGSGGSGGSGGSGGSGGSGG |
| | | SGGSLQEYEQHLRLGSVVERSHTGMP |
| 216 | SH2 cl21- | MRALVVIRLSRVTDATTSPERQLESCQQLCTQRGWDVVGVAEDLDVSGAV |
| | Zif268 | DPFDCKRRPNLARWLAFEEQPFDVIVAYRVDRLTRSVRHLQQLVHWAEDH |
| | (aa489) | KKLVVSATEAHFDTTTPFAAVVIALMGTVAQMELEAIKERSRSAAHFNIRAG |
| | | KYRGSLPPWGYLPTRVDGEWRLAPDPVQRERILEVYHRVVDNHEPLHLVAH |
| | | DLNRRGVLSPKDYFAQLRGREPQGRKWSATALKRSMISEAMLGYATLNGKT |
| | | VRGDDGAPLVRAEPILTREQLEALRAELVKTSRAKPAVSTPSLLLRVLFCAVCG |
| | | EPAYKFAGGGRKHPRYRCRSMGFPKHCGNGTVAMAEWDAFCEEQVLDLL |
| | | GDAERLEKVWVAGSDSAVELAEVNAELVDLTSLIGSPAYRAGSPQREALDAR |
| | | IAALAARQEELEGLEARPSGWEWRGTGQRFGDWWREQDTAAKNTWLRS |
| | | MNVRLTFDVRGGLTRTIDLGDLQEYEQHLRLGGGSGGSGGSGGSGGSGGS |
| | | GGSGGSERPYACPVESCDRRFSRSDELTRHIRIHTGQKPFQCRICMRNFSRS |
| | | DHLTTHIRTHTGEKPFACDICGRKFARSDERKRHTKIHLRQKDGGSGGSGGS |
| | | GGSGGSGGSGGSGGSSVVERSHTGMP |

SEQ ID NOs: 93 to 200 as shown in the sequence listing denote further fusion proteins according to the invention. The present invention also provides the DNA modifying enzymes disclosed herein and in the sequence listing without the heterologous DNA binding domain such as an integrated Zincfinger domain including any linker, as well as the respective nucleic acid sequences encoding the DNA modifying enzymes disclosed herein and in the sequence listing without the heterologous DNA binding domain.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and it is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. Any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The “percentage of sequence identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” is used herein in the context of two or more nucleic acids or polypeptide sequences, to refer to two or more sequences or subsequences that are the same, i.e. that comprise the same sequence of nucleotides or amino acids. Sequences are “identical” to each other if they have a specified percentage of nucleotides or amino acid residues that are the same. According to the present invention, at least 60% identical includes at least at least 61%, at least at least 62%, at least at least 63%, at least at least 64%, at least at least 65%, at least at least 66%, at least at least 67%, at least at least 68%, at least at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over the specified sequence, when compared and aligned for maximum correspondence over a comparison window, or designated region as measured using one of the following sequence comparison algorithms or by manual alignment and visual inspection. These definitions also refer to the complement of a test sequence. Accordingly, the term “at least XY % sequence identity” is used throughout the specification with regard to polypeptide and polynucleotide sequence comparisons. This expression preferably refers to a sequence identity of at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

In the context of the present invention, a protein having recombinase activity and comprising an amino acid sequence having at least 80% identity to a given SEQ ID NO preferably means that said protein has an amino acid sequence having at least 85%, at least 90%, at least 92%, at least 94%, at least 96%, at least 98% or at least 99% sequence identity to the given SEQ ID NO.

Likewise, in the context of the present invention, a nucleic acid sequence having at least 60% sequence identity to a given SEQ ID NO or a nucleic acid sequence reverse complementary thereto preferably means that said nucleic acid has a sequence having at least 65%, at least 70%, at least 75%, at least 80%, at least 85%, at least 90%, at least 92%, at least 94%, at least 96%, at least 98% or at least 99% sequence identity to the given SEQ ID NO or a nucleic acid sequence reverse complementary to said SEQ ID NO.

The term “sequence comparison” is used herein to refer to the process wherein one sequence acts as a reference sequence, to which test sequences are compared. When using a sequence comparison algorithm, test and reference sequences are entered into a computer, if necessary, subsequence coordinates are designated, and sequence algorithm program parameters are designated. Default program parameters are commonly used, or alternative parameters can be designated. The sequence comparison algorithm then calculates the percent sequence identities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by one of the SEQ ID NOs of the present invention, if not specifically indicated otherwise.

Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

The term “nucleic acid” and “nucleic acid molecule” are used synonymously herein and are understood as well-accepted in the art, i.e. as single or double-stranded oligo- or polymers of deoxyribonucleotide or ribonucleotide bases or both. The term “nucleic acids” as used herein includes not only deoxyribonucleic acids (DNA) and ribonucleic acids (RNA), but also all other linear polymers in which the bases adenine (A), cytosine (C), guanine (G) and thymine (T) or uracil (U) are arranged in a corresponding sequence (nucleic acid sequence). The invention also comprises the corresponding RNA sequences (in which thymine is replaced by uracil), complementary sequences and sequences with modified nucleic acid backbone or 3′ or 5′-terminus. Nucleic acids in the form of DNA are however preferred.

The term “zinc-finger (ZF) DNA-binding domain” as used herein is understood as a small protein structural motif that is characterized by the coordination of one or more zinc ions (Zn²⁺) which stabilize the fold. ZF domains contain multiple finger-like protrusions that make tandem contacts with their target molecule. The most common ZFs are Cys2His2-like fold group (C2H2) zinc-finger proteins, which have two β-sheets and one α-helix. Two cysteines in one chain and two histidines in the other chain are coordinated by the zinc ion (Cassandri et al., 2017). Further ZF groups include Gag-knuckle, treble-clef, zinc-ribbon and Zn₂/Cys₆. Each zinc finger domain contains around 30 amino acids and interacts with three nucleotide bases in the major groove of DNA. Several ZFDs can be combined together in one protein to target longer sequences. Many ZFDs are encoded in the eukaryotic genomes, specific examples of which are transcription factors Zif268 and TFIIIA. Besides, ZFDs with custom DNA sequence specificities can be engineered using numerous developed platforms and approaches known to the skilled person (Maeder et al., 2008, Kim et al., 2009, Bhakta and Segal, 2010, Sander et al., 2011, Persikov et al., 2015, Ichikawa et al., 2023; all of which are incorporated herein by reference). One specific exemplary method of engineering a ZN domain is by modular assembly as disclosed e.g. in Kim et al., 2009.

The term “transcription activator-like effector (TALE) DNA-binding domain” as used herein refers to proteins that are composed of 33-35 amino acid repeats, in which amino acids 12 and 13 determine the nucleotide specificity of the repeat. Each TALE repeat binds to one bp of a DNA sequence, and a combination of the repeats targets longer DNA sequences. Naturally occurring TALEs are secreted by plant-pathogenic bacteria Xanthomonas. TALEs with custom DNA-binding specificities can be engineered by combining the repeats in the desired order by methods well known to the skilled person (e.g. Boch et al., 2009, Bogdanove and Voytas, 2011; all of which are incorporated herein by reference).

The term “recognition site” (sometimes also referred to as “target site” or “target sequence”) as used herein refers to a specific nucleotide sequence which a DNA modifying enzyme or a DNA binding domain recognizes. In cases of recombinases being the DNA modifying enzyme, for example, the target site is the site at which DNA breakage and strand exchange occur. Such target sequences typically range between 30 and 200 base pairs in length and are comprised of two inversely repeated recombinase binding regions flanking a central spacer sequence (Meinke et al., 2016). An example of such a recognition site can be seen in the SSR Cre/loxP binding complex, where the Cre recombinase is bound to the 34 base pair loxP target sequence. The loxP recognition site comprises two 13 base pair inverted repeat Cre binding elements flanking an 8 base pair spacer region. The left half-site is the 13 base pair binding element to the left of the spacer and the right half-site is the 13 base pair binding element to the right of the spacer. Depending on the number and relative orientation of the recognition sites and their spacers, the DNA recombining enzyme either performs an excision, an integration, an inversion or a replacement of genetic content (reviewed in Meinke et al., 2016). Therefore, according to a preferred embodiment, a “recognition site” is a nucleotide sequence comprising a first half-site, a second half-site, and a spacer separating the first and the second half-site. For a recombination event to occur, a recombinase enzyme complex recognizes a first recognition site and a second recognition site on a DNA double strand. The recognition sites are also referred to as upstream and downstream recognition sites, depending on their location on the DNA double strand.

Target sites of DNA binding domains are known in the art and include for example GCGTGGGCG (SEQ ID NO: 1) for Zif268 (Christy and Nathans, 1989), (T/A)(G/A)CAGAA(T/G/C) (SEQ ID NO: 2) for ZNF217 (Nunez et al., 2011), and TATAAACCCCCTCCAACCAGGTGCTAA (SEQ ID NO: 3) for AvrXa7 TALE (Richter et al., 2014).

In symmetric recognition sites, the first half-site (e.g. the left half-site) and the second half-site (e.g. the right half-site) are identical and palindromic (reverse complement). In asymmetric recognition sites, the first half-site (e.g. the left half-site) and the second half-site (e.g. the right half-site) are not identical and not palindromic, i.e. they differ from each other in at least one nucleotide.

As used throughout the present disclosure, the amino acid position of a DNA modifying enzyme for insertion of a heterologous DNA binding domain denotes the amino acid position in the DNA modifying enzyme which precedes the heterologous DBD to be inserted, that is the identified position is directly N-terminal of the DBD or a respective linker, and the DBD or respective linker follows C-terminal of said identified position. In some cases, two positions are indicated between which the heterologous DBD or respective linker(s) are to be inserted. For example, if position G278 is identified, this means that the heterologous DBD or a respective linker follows directly after (i.e. C-terminal) of said position G278. If the position is indicated to be between and positions G278 and S279, this means that the heterologous DBD or a respective linker follows directly after (i.e. C-terminal) of said position G278 and ends directly before (i.e. N-terminal) of S279 as exemplified in the following: G278-(linker)-DBD-(linker)-S279.

The term “x-derived recombinase” wherein x denotes a specific recombinase such as but not limited to Cre or Vika, as used herein refers to a recombinase that used the cited recombinase as the starting point for engineering, either by directed molecular evolution or by protein engineering. An x-derived recombinase thus shows at least 50% sequence identity with the recombinase from which it is derived, preferably at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the recombinase from which it is derived. The term “x-type recombinase” is used interchangeably with “x-derived recombinase” herein. The same definitions likewise apply to all proteins or polypeptides derived from a specified protein or polypeptide mentioned herein.

The term “therapeutically effective amount” as used herein means that amount of active compound or pharmaceutical agent that elicits the biological or medicinal response in a tissue system, animal or human being sought by a researcher, veterinarian, medical doctor or other clinician, which includes alleviation of the symptoms of the disease or disorder being treated.

The term “pharmaceutical composition” as used herein refers to a substance and/or a combination of substances being used for the identification, prevention or treatment of a disease or tissue status. The pharmaceutical composition is formulated to be suitable for administration to a patient in order to prevent and/or treat a disease. Further a pharmaceutical composition refers to the combination of an active agent with a carrier, inert or active, making the composition suitable for therapeutic use. Such a carrier is also referred to as being pharmaceutically acceptable. Pharmaceutical compositions can be formulated for oral, parenteral, topical, inhalative, rectal, sublingual, transdermal, subcutaneous or vaginal application routes according to their chemical and physical properties. Pharmaceutical compositions comprise solid, semisolid, liquid, transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the carrier systems of the present invention. Semisolid compositions that can be used in the context of the invention comprise emulsion, suspension, creams, lotions, gels, globules, buccal tablets and suppositories.

As used herein, the term “pharmaceutically acceptable” embraces both human and veterinary use: For example, the term “pharmaceutically acceptable” embraces a veterinary acceptable compound or a compound acceptable in human medicine and health care.

The term “subject” as used herein, refers to an animal, preferably a mammal, most preferably a human.

### Description of Embodiments

The present disclosure shows that insertions of a DNA binding domain (DBD) into a DNA modifying enzyme renders its activity conditional for binding of the DBD to its respective target sequence. Thus, according to a first aspect, the present invention provides a method for identifying an amino acid position of a DNA modifying enzyme for insertion of a heterologous DBD. The method comprises the steps of:
(i) providing a library of DNA modifying enzymes, wherein the members of the library comprise heterologous amino acid sequence insertions throughout the DNA modifying enzyme;
(ii) identifying those DNA modifying enzymes of the library that have DNA modifying activity; and
(iii) identifying the position of the insertion in those DNA modifying enzymes identified in step (ii).

According to a preferred embodiment, the method of the present invention identifies those positions in a DNA modifying enzyme, at which the DBD can be inserted for rendering the DNA modifying enzyme active on its target site only when the heterologous DBD also binds to the DBDs target site. In other words, the DNA modifying enzyme is essentially inactive in those cases, in which the DNA modifying enzyme binds to its target site and in which the DBD does not bind to the respective DBD target site. The DNA modifying enzyme is, however, active in those cases, in which the DNA modifying enzyme binds to its target site and in which the DBD also binds to the respective DBD target site. The method of the invention thus allows identifying positions within the DNA modifying enzyme for insertion of the DBD for rendering the enzyme's activity conditional on the binding of the DBD to the respective DBD target site.

The DNA modifying enzyme can be any DNA modifying enzyme known in the art, such as but not limited to a recombinase, e.g. a site-specific recombinase (such as a serine and tyrosine site-specific recombinase), a large serine recombinase (integrase), a transposase (such as PiggyBAC transposase), or a topoisomerase. In accordance with one preferred embodiment of the present invention, the DNA modifying enzyme is preferably a recombinase, more preferably a tyrosine recombinase, and most preferably a tyrosine recombinase selected from the group consisting of Cre and Cre-derived recombinases, Vika (disclosed e.g. in EP 2690177 A1 and incorporated herein by reference in its entirety), Panto (disclosed e.g. in EP 3263708 A1 and incorporated herein by reference in its entirety), Dre, D7L, D7R, Nigri (disclosed e.g. in EP 2877585 A1 and incorporated herein by reference in its entirety), VCre, SCre, YR1, YR2, YR4, YR6, YR8, YR9, YR11, YR12 (Jelicic et al. 2023), Tre, Brec and recombinases derived therefrom. According to a further preferred embodiment, the DNA modifying enzyme is an engineered site-specific variant of a naturally occurring DNA recombinase. According to a further preferred embodiment, the DNA modifying enzyme is selected from the group consisting of A118, TP901, φRV1 (also termed PhiRv1), φC31, R4, Wβ, Tnpx, Cp36, Dn29, Kp03, Nm60, Pa01, Si74 lambda-Int, Flp, Kd, Kw, B2, and B3, piggyBac, sleeping beauty, topoisomerases, Bxb1 and enzymes derived therefrom. According to a further preferred embodiment, the DNA modifying enzyme is selected from the group consisting of Bxb1, PhiC31, Sh25, Si74, Bm99, Me99, Ma37, Nm60, Cc91, Vh19, Cs56, Bt24, No67, Fm04, Bu30, Ma05, Rh64, Cb16, uCb4, Ec03, Ec04, Ec05, Ec06, Ec07, Ef01, Ef02, Kp01, Kp03, Kp04, Kp05, Pa01, Pa03, Sa01, Sa02, Pf13, Td08, Se37, Ct03, Cd31, Ps40, Sa10, Td01, Enc3, Fp10, Ph43, Sm18, Cd16, Pf80, Bs46, Pf48, Rb27, Sa51, Bc30, Cd04, Cd15, Sa34, Pp20, R109, Efs2, Pf15, Ps45, Sp56, Dn29, Vh73, Em12, Pc64, Vp82, Cp36, Pc01, Enc9 (Durrant et al., 2023). According to a particularly preferred embodiment, the DNA modifying enzyme is a site-specific recombinase or a large serine recombinase (integrase).

In accordance with the present invention, the position for inserting the DBD (with or without one or more linkers) into the amino acid sequence of the DNA modifying enzyme—with reference to the amino acid sequence of the DNA modifying enzyme—follows a first amino acid position of the DNA modifying enzyme and ends before a second amino acid position of the DNA modifying enzyme directly following said first amino acid position in N- to C-terminal direction of the DNA modifying enzyme. In the present disclosure, either said first amino acid position is referred to (meaning that the insertion is directly after said first amino acid position and ends before the amino acid position immediately following said first amino acid position and which is not explicitly referred to), or it is explicitly specified between which two positions the DBD is inserted. Thus, for example and in accordance with a preferred embodiment of the present invention, the insertion of a DBD at position 278 is between amino acid positions 278 and 279 with respect to the DNA modifying enzyme such as Cre-derived recombinase Brec1 having SEQ ID NO: 4. This means that the amino acid sequence of the DBD either directly, or via a linker, starts immediately after said position 278, and ends directly, or via a linker, immediately before position 279 of SEQ ID NO: 4, giving rise to e.g. the construct as shown in SEQ ID NO: 5 (Brec1²⁷⁸-Zif268) with a zinc-finger DBD inserted between positions G278 and S279 of Brec1.

The DNA binding domain (DBD) is heterologous to the DNA modifying enzyme, meaning that it is not a DBD of the DNA modifying enzyme itself. The DBD can be any DBD known in the art, such as but not limited to zinc-fingers, TAL effectors (Boch et al., 2009), Cas proteins (such as dCas9 (Jinek et al., 2016), dCas12 (also called Cpf1) (Zetsche et al., 2015), preferably dead Cas proteins (Xu and Qi, 2019)), DBDs of artificial transcription factors such as helix-turn-helix proteins (such as LexA (Schnarr et al., 1991) and Lac (Matthews and Nichols, 1997)), and leucin-zipper coiled-coil (such as GCN4 (Landschulz et al., 1988)). In accordance with one preferred embodiment of the present invention, the DBD is preferably selected from the group consisting of a zinc-finger DBD and a transcription activator-like effector (TALE) DBD.

The library of DNA modifying enzymes comprising heterologous amino acid sequence insertions throughout the DNA modifying enzyme can be created using any means known in the art for introducing amino acid sequences such as small peptides into the DNA modifying enzyme. The construction of such libraries is well known in the art and to the skilled person. Preferably, the library is constructed using pentapeptide scanning mutagenesis (Hayes and Hallet, 2000) and preferably takes place at the DNA level. Thus, according to a preferred embodiment of the present invention, the library of DNA modifying enzymes provided in step (i) of the method of the present invention is encoded by a nucleic acid library. A preferred method for generating such a library is the use of Mu transposition (Haapa et al., 1999). Such random transposition can be performed in vitro by MuA transposase, in the presence of the plasmid carrying the target gene and Entranceposon—a DNA fragment that is intended to be integrated into the sequence of the target gene, which includes a selection gene (antibiotic resistance), rare-cutting restriction site of a rare-cutting restriction enzyme NotI, and sites for MuA complex assembly on both ends. The insertion clones generated in the transposition reaction are subsequently digested with NotI to remove the body of the Entranceposon. Closure of the NotI digested clones by self-ligation results in a 15 bp insertion in the target DNA, which is subsequently translated into five extra amino acids.

The amino acid sequence insertions are preferably small peptides of a specific length in order to ensure comparability of the results. The length of such peptides is preferably between 3 and 15 amino acids, such as 3, 4, 5, 6, 7, 8, 9, 10, 11, 12, 13, 14, and 15, more preferably between 3 and 10, even more preferably between 4 and 8 amino acids, and most preferably between 4 and 6 amino acids. According to a particularly preferred embodiment, the amino acid sequence insertions each have a length of 5 amino acids. The sequence of said insertional amino acids or small peptides is not particularly limited. In order to ensure better comparability, the length is preferably identical for all amino acid sequences or small peptides inserted into the DNA modifying enzymes in the library. The amino acid sequence can be any random amino acid sequence.

For identifying those DNA modifying enzymes of the library that have DNA modifying activity, any method known in the art can be used. For example and according to a preferred embodiment of the present invention, the library of DNA modifying enzymes of step (i) is cloned into suitable expression vectors comprising the respective target site(s) of the DNA modifying enzyme, the enzyme is expressed, and it is determined whether or not the expressed enzyme modified the DNA sequence in the vector at or between the target site(s). The determining step preferably includes isolating plasmid DNA and sequencing of the isolated plasmid DNA or restriction digestion of the isolated plasmid DNA.

The cloning into suitable expression vectors can be done by conventional methods such as digesting the coding nucleic acid using suitable restriction enzymes and ligating the coding nucleic acid into the expression vector. The expression vector to be used for the library can be any expression vector considered useful by the person of ordinary skill in the art. A preferred expression vector to be used in the context of the present invention is the pEVO expression vector described in Buchholz and Stewart 2001 or a modified version thereof described herein. The expression vector preferably comprises at least two regions. In the first region, the expression vector comprises the nucleic acid sequence encoding the DNA binding domain with the amino acid insertion. The second region preferably comprises at least two target sites of the DNA modifying enzyme. The second region on the expression vector is separated from the first region in that both regions do not overlap. The expression vectors containing the nucleotide sequences encoding the DNA modifying enzyme with the integrated amino acid sequences or small peptides and the target sites are preferably introduced into suitable (host) cells. Any suitable eukaryotic or prokaryotic (host) cell can be used that allows expression of the encoded protein. Preferred cells are bacterial cells, particularly preferred cells are cells of Escherichia coli. According to a particularly preferred embodiment, the (host) cells are XL-1 Blue E. coli cells, and the ligated plasmids are introduced via electroporation of the cells. The skilled person is well aware about alternative suitable methods for introducing a ligated plasmid into a host cell for subsequent expression of the encoded protein. The host cells carrying the library of expression vectors are preferably cultured to allow the expression of the encoded DNA modifying enzymes. The culturing conditions are not particularly limited and will be selected by the skilled person based on the host cells used. For example, in case of using XL-1 Blue E. coli cells, it is preferred to culture the transformed bacteria in LB medium at 37° C. Conditions for introducing expression of the encoded LSR variants also depend on the host cells and plasmid vectors used. In the case of using pEVO expression vectors and XL-1 Blue E. coli cells, expression can be induced by adding e.g. arabinose to the culture medium.

Upon expression of the encoded DNA modifying enzyme comprising the integrated amino acid sequences or small peptides, those DNA modifying enzymes which are active will start modifying the nucleic acid sequence of the expression vector at or between the target site(s). For determining whether the expressed DNA modifying enzyme is active, plasmid DNA of these cultures after cultivation and induction of the expression is preferably isolated using any suitable method known to the person or ordinary skill in the art. The isolated plasmid DNA can then be analysed for activity of the DNA modifying enzyme comprising the integrated amino acid sequences or small peptides on the target sites. For example, at least the respective portion of the plasmid DNA encoding the target site(s) and any nucleotides in between two target sites can be sequenced. Such sequencing may also include the first region of the expression vector encoding the DNA modifying enzyme. Alternatively, the plasmid DNA may be digested using one or more restriction enzymes. For example, restriction digestion can be performed on the sequence of the expression vector comprising the target site(s), followed by analysis of the digestion fragments. The restriction enzyme is preferably selected so as to excise also the portion of the plasmid encoding the DNA modifying enzyme, leading to different sizes of the fragment depending on whether the DNA modifying enzyme is active or inactive. One or more different restriction enzymes can be used for his purpose. The restriction enzyme(s) is preferably selected so as to excise the portion of the plasmid encoding the target sites and optionally the DNA modifying enzyme, leading to a larger fragment including the sequence in between the target sites in case of no reaction/activity such as an excision reaction by the DNA modifying enzyme, and to a smaller fragment in case of a reaction/activity such as an excision reaction by the DNA modifying enzyme between the target sites. The size difference can then be visualized for example in gel electrophoresis. The visualizations can further be analysed for the relative amount of large and small fragments, allowing the calculation of a percentage value for recombined and non-recombined (excised and non-excised) plasmids. Band intensity values can for example be divided by the combined values of the recombined and non-recombined bands to determine the fraction of modified DNA, which can be converted to a percentage value by multiplying with 100.

An alternative method for determining whether or not a DNA modifying enzyme is active on the target site(s) in the expression vector is by analysing the nucleic acid sequence of the vector in isolated plasmids. This can be done by PCR amplification of the region comprising the target site(s) and optionally the region encoding the DNA modifying enzyme, and/or by sequencing the region comprising the target site(s) and optionally the region encoding the DNA modifying enzyme. In cases of for example an excision reaction by an active the DNA modifying enzyme, the sequence will be shorter, lacking the excised sequence between the target sites.

According to a preferred embodiment, an active DNA modifying enzyme exhibits at least about 5% activity, about 6%, about 7%, about 8%, about 9%, about 10%, preferably at least about 15%, about 20%, about 25%, about 30%, about 35%, about 40%, about 45%, or at least about 50% activity on the respective target site(s). According to a preferred embodiment, DNA modifying enzymes that show less than about 1%, less than about 2%, less than about 3%, less than about 4%, or less than about 5% activity on its target site(s), is considered as being inactive on the respective target site(s).

Further methods for determining the activity of a DNA modifying enzyme are well known in the art. One such further and preferred method for determining activity of a DNA modifying enzyme comprises digesting recombined and non-recombined plasmids from the culture of host cells with restriction enzymes as explained herein. The differences in plasmid sizes of the resulting DNA fragments depending on the activity of the DNA modifying enzyme containing the plasmid backbone also show a difference in size can be visualized for example in agarose gel electrophoresis. The visualizations can then be analysed for the relative amount of large and small fragments, allowing the calculation of a percentage value for recombined and non-recombined plasmids. Band intensity values can for example be divided by the combined values of the recombined and non-recombined bands to determine the fraction of recombined DNA, which can be converted to a percentage value by multiplying with 100. Thus, the method of the present invention preferably further comprises the step of determining an activity rate for each insertion variant of the DNA modifying enzyme generated by the method of the present invention.

Those DNA modifying enzymes that turned out not to be active are preferably removed from the library. Thus, according to one embodiment of the present invention, the method further comprises the step of removing inactive DNA modifying enzymes from the library. An exemplary method of removing inactive DNA modifying enzymes is analogous to the restriction digestion method for determining whether the DNA modifying enzyme is active on the respective target sites. In cases of two target sites, the restriction enzyme(s) digest the plasmid between the target sites. In the absence of a DNA modifying reaction, the restriction enzyme will cut the vector in between the two target sites and the vector will be linearized. If the DNA modifying enzymes is active, the respective portion between the two target sites will be modified (e.g. removed, exchanged or a nucleotide sequence will be inserted) and no restriction digestion that could linearize the plasmid will occur. PCR primers can be selected so as to allow amplification of the region of the vector comprising the sequence encoding the DNA modifying enzymes and the region of the vector comprising the target site(s), wherein the PCR primers point at each other. If a DNA modifying reaction took place caused by an active DNA modifying enzyme, any restriction site is preferably removed and the vector stays intact, preserving correct orientation the PCR primers and thus amplification of a product including both regions on the plasmid. Following restriction digestion, the vectors carrying the active DNA modifying enzymes are preferably transformed again into E. coli, and the plasmids are isolated after culturing the cells. The obtained plasmids can then be digested with restriction enzymes that cut upstream and downstream of the DNA modifying enzyme, and the fragment carrying the DNA modifying enzyme can then be separated from the backbone by gel electrophoresis, preferably followed by preparation of the DNA fragments. In addition or alternatively, DNA plasmids or fragments encoding the active DNA modifying enzymes can be sequenced.

According to a preferred embodiment, the method for identifying an amino acid position of a DNA modifying enzyme for insertion of a heterologous DBD comprises cloning of the respective DNA modifying enzyme(s) into a suitable vector (preferably the pEVO vector), and combining/mixing the vectors with MuA transposase and Entranceposon for a transposition reaction resulting in a library, in which the Entranceposon is randomly inserted within the recombinase sequence. In a next step, the inserted Entranceposon is excised by a restriction enzyme digest, resulting in a library of recombinase mutants carrying the amino acid insertions. The obtained library is cloned into suitable plasmids (preferably pEVO) carrying the respective target sites of the DNA modifying enzyme for selection. Upon expression of the recombinases, the mutated variants that retain their enzymatic activity on the target sites are selected by digestion with a unique restriction enzyme and are sequenced.

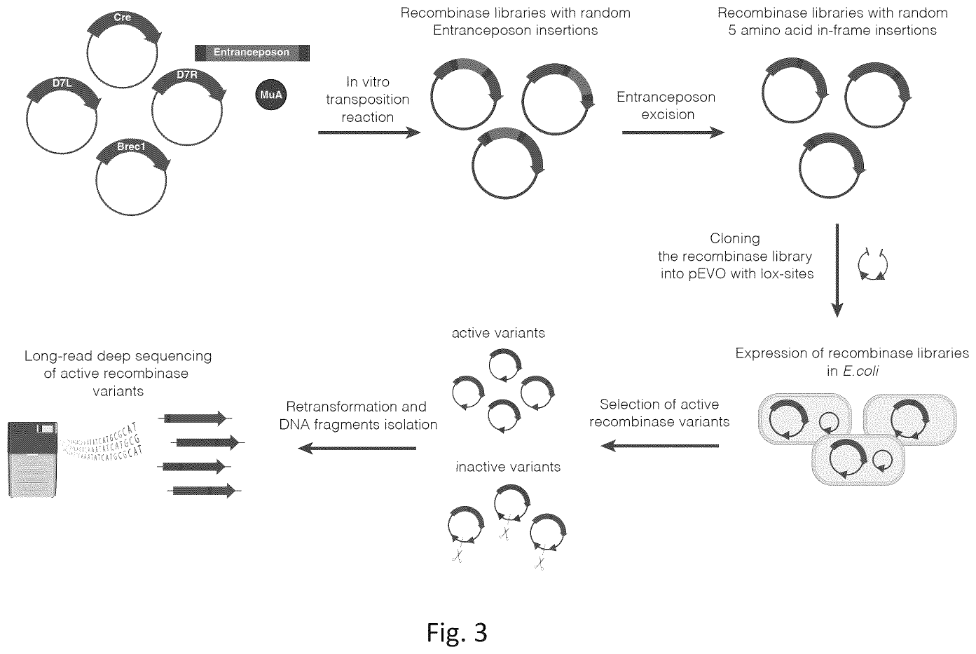

An exemplary and preferred embodiment of the present invention is shown in FIG. 3, according to which a library of DNA modifying enzymes (in the example shown in FIG. 3 the DNA modifying enzyme is a Cre-type recombinase) is generated by cloning the sequence encoding the recombinase into the expression vector pEVO, which is mixed with the MuA transposase and Entranceposon in vitro for a transposition reaction resulting in a library, in which the Entranceposon is randomly inserted within the recombinase sequence. In a next step, the inserted Entranceposon is excised by a restriction enzyme digest, resulting in the library of recombinase mutants carrying insertions for five random amino acids. The obtained library is cloned into pEVO plasmids carrying the respective recombinase target sites (here lox sites) for selection. Upon expression of the recombinases, the mutated variants retaining recombination activity on the target sites were selected by digestion with a unique restriction enzyme (depicted as scissors) and sequenced with PacBio long-read deep sequencing.

According to a further embodiment of the present invention, the method for identifying an amino acid position of the DNA modifying enzyme for insertion of a heterologous DBD further comprises the step of mapping the one or more positions of the insertions identified in the method according to the present invention to structural data of the DNA modifying enzyme.

This mapping is preferably performed by identifying the amino acid position in the 3D structure or in a prediction of the 3D structure of the DNA recombining enzyme bound to a DNA, which corresponds to the one or more positions of the insertions identified in the method according to the present invention.

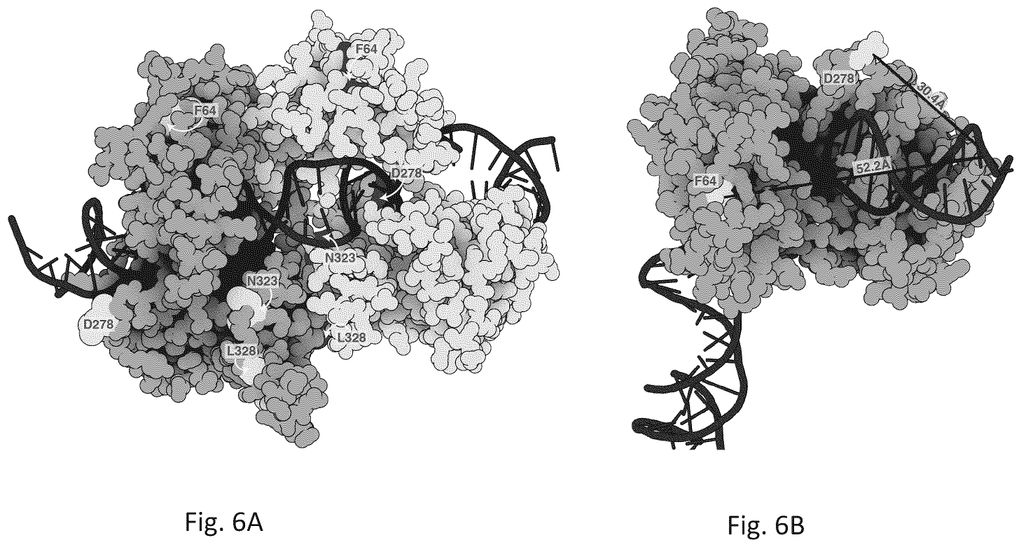

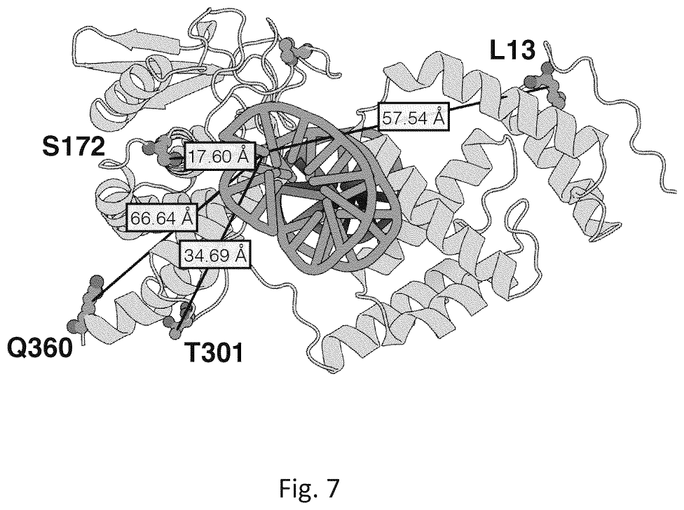

Such mapping is exemplarily shown in FIG. 6A, displaying a 3D drawing of a Cre-type recombinase based on PDB ID 1Q3U disclosed in Ennifar E., 2003, and the positions identified in the recombinase by the method of the present invention. If no such structural data is available for the respective DNA modifying enzyme, the structure of said DNA modifying enzyme can be predicted by using respective programs or databases known in the art to perform predictions of protein structure, such as AlphaFold and AlphaFold2. This predicted structural data can further be superimposed with structural data of a known DNA modifying enzyme, preferably bound to DNA, such as the one shown in FIG. 6A for a Cre-type recombinase. FIG. 7 shows such a prediction and analysis of the 3D protein structure of Vika recombinase, and FIG. 26 shows the 3D protein structure of Bxb1. The 3D model of Vika and Bxb1 wildtype recombinases was predicted using AlphaFold (Mirdita et al., 2022). The predicted model of Vika was superimposed with the monomer of the Cre/loxP synapse pre-cleavage complex (PDB ID 1Q3U, Ennifar E., 2003). The most frequent positions that tolerated insertions in the pentapeptide scanning mutagenesis of the Vika-type recombinase are highlighted, and the respective amino acid at said position is indicated in FIG. 7. The predicted model of Bxb1 was superimposed with the C-terminal domain of serine integrase A118 (SEQ ID NO: 204) bound to an attP DNA half-site (SEQ ID NO: 217 (attP(A118)) (PDB ID 4KIS (Rutherford, 2013). The most frequent positions that tolerated insertions in the pentapeptide scanning mutagenesis of the Bxb1-type recombinase are shown in FIG. 26.

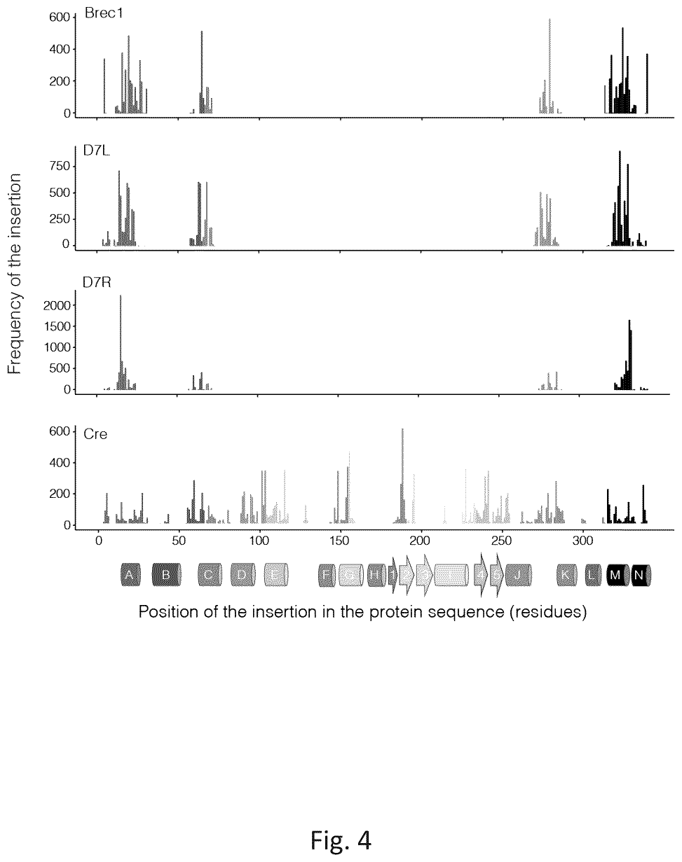

According to a further embodiment of the present invention, positions for insertions can be selected based on the frequency of appearance in the method of identifying an amino acid position. For example, the library of DNA modifying enzymes in a high-throughput method of the present invention is based on multiple random integrations at multiple random positions in the respective DNA modifying enzyme. If during evaluation of the activity of the DNA modifying enzymes comprising the respective insertion an insertion position resulting in an active DNA modifying enzyme is identified more frequently than other positions, such position can be selected for the insertion. A respective chart showing frequency of insertion positions resulting in an active DNA modifying enzyme is shown in FIG. 4, displaying frequencies and distribution of the peptide insertions in the sequences of Brec1, D7L, D7R and Cre recombinases. FIG. 4 further shows the secondary structure elements below the graph, with alpha-helices displayed as cylinders with letters and beta-sheets represented as numbered arrows. In the examples shown, most insertions are tolerated in Brec1, D7L and D7R at positions near the N-terminus and the C-terminus between helices B and C, and between helices J and K, while tolerated positions in Cre recombinase can be found throughout the enzyme.

According to one embodiment of the present invention, the method further comprises the step of selecting one or more amino acid positions for insertion of the heterologous DBD that are surface exposed in the DNA modifying enzyme. Preferably, these surface exposed positions are further in proximity to the DNA binding site of the DNA modifying enzyme. Whether or not an identified amino acid position is surface exposed can be determined by using available structural data of the DNA modifying enzyme or by predicting the structure thereof as described herein. The proximity to the DNA binding site of the DNA modifying enzyme can be determined e.g. by simulating binding of the DNA modifying enzyme to a DNA molecule comprising its target site using respective programs or databases known in the art to perform predictions of protein structure, such as AlphaFold and AlphaFold2. This predicted structural data can further be superimposed with structural data of a known DNA modifying enzyme, preferably bound to DNA, such as the one shown in FIG. 6A for a Cre-type recombinase. According to the present invention, an amino acid position is in proximity to the DNA binding site of the DNA modifying enzyme if it is within about 1 to about 50 Å, preferably within about 5 and about 45 Å, more preferably within about 10 and about 40 Å, even more preferably within about 15 and about 35 Å, most preferably within about 20 and about 30 Å of the DNA binding site or of DNA bound by the DNA modifying enzyme as determined based on available or predicted structural data of the DNA modifying enzyme. Preferably, said structural data or prediction data includes a DNA bound by said DNA modifying enzyme. FIGS. 6B and 7 show respective illustrations of Cre-type recombinase (FIG. 6B) and of Vika recombinase (FIG. 7) with DNA bound by the enzyme and the distances of potential insertion positions identified by the method of the present invention to the DNA binding site of the DNA modifying enzyme. According to FIG. 6B, position D278 identified to tolerate an insertion of heterologous (poly)peptides or proteins, has a distance to the DNA binding site of Cre recombinase of about 30 Å, while position F64 has a distance of about 52 Å. In this case, position D278 is preferred over position F64, since D278 is exposed on the surface of the enzyme and is thus more eligible for attaching any additional peptides or proteins, and it is nearer to the DNA binding site compared to position F64. FIG. 7 shows positions for insertions identified in Vika recombinase. Position S172 was preferred over L13, T301 and Q360 for being closer the DNA binding site of Vika recombinase. It is, however, to be noted that the distance to the DNA binding site is a purely optional parameter that can be used for identifying respective positions for insertions in a DNA modifying enzyme.

The present invention further provides a method for producing a DNA modifying enzyme comprising an insertion of a heterologous DBD. According to the present invention, this method comprising the steps of (i) inserting a nucleic acid sequence encoding the heterologous DBD into a nucleic acid sequence encoding the DNA modifying enzyme at one or more nucleotide triplet(s) encoding the one or more positions identified in the method of the present invention and as detailed herein, and of (ii) expressing the nucleic acid sequence produced in said step (i). The nucleic acid sequence encoding the heterologous DBD can be introduced into the nucleic acid sequence encoding the DNA modifying enzyme using any suitable method known in the art, such as by standard cloning techniques, for example using primers to introduce restriction sites and cloning of the DBD via the restriction sites. A preferred method includes golden-gate assembly as described in e.g. Engler et al., 2008. Inserting a nucleic acid sequence encoding the heterologous DBD into a nucleic acid sequence encoding the DNA modifying enzyme at one or more nucleotide triplet(s) encoding the one or more positions identified in the method of the present invention means that the nucleic acid sequence encoding the heterologous DBD is inserted directly after (preferably 3′ of) the nucleotide triplet encoding the respective identified amino acid position, preferably in-frame with the nucleotide triplet and thus in-frame with the coding sequence encoding the DNA modifying enzyme.

In accordance with a preferred embodiment of the present invention, the DBD inserted at the respective position further comprises an N-terminal and/or C-terminal peptide linker, preferably an N-terminal and a C-terminal peptide linker. According to a particularly preferred embodiment, the peptide linker is a flexible peptide linker such as a linker comprising glycine and serine residues. Such flexible linkers are not particularly limited and are known in the art, such as in WO 2021/110846, incorporated herein by reference in its entirety. Particularly preferred linkers include (G2S)ₙ and (G3S)ₙ linkers with n being an integer of between 1 and 10, preferably 2, 3, 4, 5, 6, 7, 8, or 9, more preferably of between 7 and 9 and most preferably n=8. The glycine/serine linkers may further comprise one, two, three, four or five mutations from glycine to arginine (G→R). According to a preferred embodiment, such mutations are present only once per GS building block of the linker. For example, such a linker may have the sequence (GGS)₆-GRS-GGS (SEQ ID NO: 6) or (GGS)₃-GRS-GGS-GRS-(GGS)₂ (SEQ ID NO: 7). A particularly preferred linker is (GGS)₆-GRS-GGS (SEQ ID NO: 6). Such a glycine/serine linker comprising at least one mutation from glycine to arginine is preferably positioned at the C-terminus of the DBD to be inserted into the DNA modifying enzyme. The present disclosure shows that such a mutation in the linker further increases recombination efficiency of the DNA modifying enzyme (cf. Example 4). According to a particular embodiment of the present invention, the most preferred linker (GGS)₆-GRS-GGS (SEQ ID NO: 6) is used at the C-terminus of the inserted DBD, while a (GGS)₈ linker is used at the N-terminus of the inserted DBD. The linker(s) may further comprise one or more restriction sites allowing cutting out of parts of the linker and/or the entire DBD. The restrictions sites are preferably located at or near the N- and/or C-terminus of the linker.

In accordance with the method for producing a DNA modifying enzyme, the nucleic acid sequence encoding the heterologous DBD further comprises a nucleic acid sequence encoding such peptide linker upstream and/or downstream of the nucleic acid sequence encoding the heterologous DBD, preferably upstream and downstream of the nucleic acid sequence encoding the heterologous DBD. When inserting such a DNA at a nucleotide triplet encoding the position identified in the method of the present invention, the nucleic acid sequence encoding the DNA modifying enzyme and the respective linker(s) are preferably inserted in-frame with and directly after the (preferably 3′ of) nucleotide triplet and thus in-frame with the coding sequence encoding the DNA modifying enzyme.

According to a particularly preferred embodiment of the present invention, the DBD is preferably a zinc-finger DBD or TALE DBD as defined herein.

According to a further particularly preferred embodiment, the DNA modifying enzyme is a transposase or a recombinase, preferably a serine recombinase or a tyrosine recombinase, more preferably a tyrosine recombinase.

According to a further aspect, the present invention provides a DNA modifying enzyme comprising an insertion of a heterologous DBD. Preferably, the DNA modifying enzyme comprising an insertion of a heterologous DBD is obtained by or is obtainable by a method according to present invention and as described herein. According to a particularly preferred embodiment, the present invention provides a DNA modifying enzyme comprising an insertion of a heterologous DBD, wherein the DNA modifying enzyme is Cre and the insertion is at position 3, 4, 5, 6, 11, 12, 13, 14, 15, 16, 17, 19, 20, 21, 22, 23, 24, 25, 26, 27, 28, 29, 30, 38, 41, 42, 43, 54, 55, 56, 57, 58, 59, 60, 61, 62, 63, 64, 65, 67, 68, 69, 70, 71, 72, 74, 75, 77, 78, 80, 81, 88, 89, 90, 91, 92, 94, 95, 96, 97, 98, 101, 102, 103, 104, 105, 106, 107, 108, 109, 110, 111, 112, 113, 114, 115, 116, 117, 127, 128, 129, 143, 144, 146, 147, 148, 149, 150, 151, 152, 153, 154, 155, 156, 157, 158, 159, 160, 182, 183, 184, 185, 186, 187, 188, 189, 190, 191, 192, 194, 195, 196, 198, 199, 213, 214, 215, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 261, 262, 263, 265, 266, 267, 268, 270, 271, 272, 273, 274, 275, 277, 278, 279, 280, 282, 283, 284, 285, 286, 287, 288, 299, 300, 301, 302, 312, 313, 315, 316, 317, 319, 320, 321, 322, 323, 324, 325, 326, 327, 328, 329, 330, 331, 332, 336, 337, 338, or 339, preferably with respect to SEQ ID NO: 201 or a sequence at least 80% identical thereto. According to a particularly preferred embodiment, the present invention provides a DNA modifying enzyme comprising an insertion of a heterologous DBD, wherein the DNA modifying enzyme is Cre or a Cre-derived recombinase and the DBD is inserted at position 4, 14, 15, 16, 17, 19, 20, 22, 23, 59, 63, 64, 67, 68, 188, 274, 275, 278, 280, 283, 319, 320, 322, 323, 324, 325, 326, 327, 328 or 329, preferably with respect to SEQ ID NO: 4, 17, 18 or 201, or a sequence at least 80% identical thereto. According to a particularly preferred embodiment, the insertion is between amino acid positions 278 and 279, preferably between D278 and S279. The numbering of amino acid positions 278 and 279 with respect to the Cre or a Cre-derived recombinase preferably refers to SEQ ID NO: 4, 17, 18 or 201, or a sequence at least 80% identical thereto. According to a further particularly preferred embodiment, the present invention provides a DNA modifying enzyme comprising an insertion of a heterologous DBD, wherein the DNA modifying enzyme is Vika and the insertion is at position 2, 3, 4, 5, 6, 7, 8, 9, 10, 11, 13, 14, 15, 19, 21, 22, 24, 31, 32, 34, 37, 67, 117, 167, 168, 169, 170, 171, 172, 173, 174, 175, 176, 252, 259, 260, 261, 262, 263, 264, 265, 266, 267, 269, 270, 271, 272, 273, 274, 275, 276, 277, 278, 279, 280, 296, 297, 298, 299, 300, 301, 302, 303, 304, 305, 306, 349, 354, 356, 358, 359, 360 or 361, preferably with respect to SEQ ID NO: 8 or a sequence at least 80% identical thereto. According to a particularly preferred embodiment, the present invention provides a DNA modifying enzyme comprising an insertion of a heterologous DBD, wherein the DNA modifying enzyme is Vika or a Vika-derived recombinase and the DBD is inserted at position 2, 6, 8, 10, 13, 168, 170, 171, 172, 175, 258, 259, 260, 261, 262, 263, 264, 265, 266, 267, 269, 270, 271, 272, 273, 274, 275, 276, 277, 280, 297, 299, 300, 301, 302, 303, 304, 305, 358, 359, 360 or 361 preferably with respect to SEQ ID NO: 8, 14, 15 or 16, or a sequence at least 80% identical thereto. According to a particularly preferred embodiment, the insertion is between amino acid positions 172 and 173, preferably between S172 and V173. The numbering of amino acid positions 172 and 173 with respect to a Vika or a Vika-derived recombinase preferably refers to SEQ ID NO: 8. Corresponding amino acid positions to those identified with respect to a specific amino acid sequence can be easily identified in other proteins or enzymes such as recombinases by aligning the sequence of the reference enzyme or protein with the sequence of the enzyme or protein in which the corresponding amino acid shall be identified. Thus, the term “an amino acid position corresponding to position . . . ” and similar expressions as used in the context of the present invention refer to the position of the amino acid that aligns in an alignment of amino acid sequences with an amino acid sequence of an enzyme or protein described herein. One specific example of such a corresponding amino acid is the position G278 in Brec (SEQ ID NO: 4), which aligns with position D278 of Cre recombinase (SEQ ID NO: 201).

The heterologous DBD optionally comprises at its N- and/or C-terminus a peptide linker as described herein.

The present invention further provides a DNA modifying enzyme comprising a heterologous DNA binding domain inserted therein, wherein the protein comprises an amino acid sequence having at least 80% sequence identity to any one of SEQ ID NOs: 5, 9 to 13, 19, 20, 22, 30, 41, 42, 51, 64 and 91 to 200.

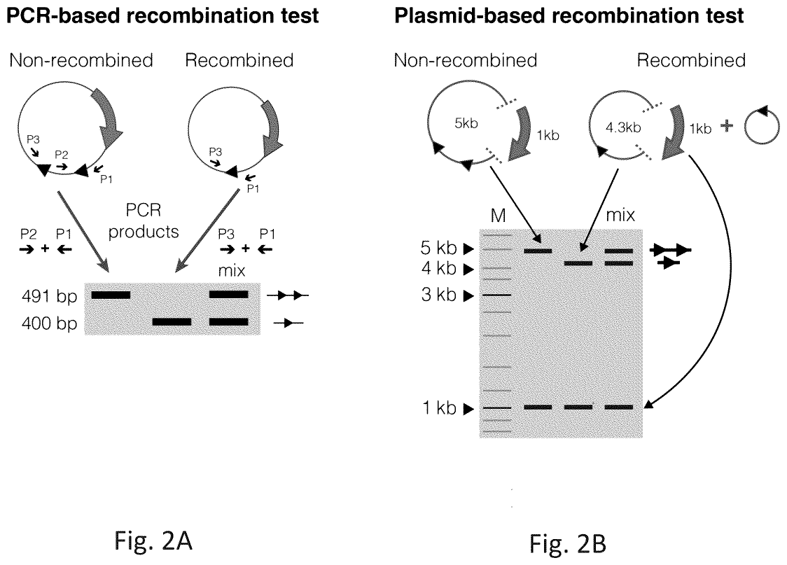

According to a further aspect, the present invention provides a DNA modifying enzyme as described herein comprising an insertion of a heterologous DBD, wherein the DNA modifying enzyme is essentially inactive on its target site when the heterologous DBD does not bind to its target DNA, and wherein the DNA modifying enzyme is essentially active on its target site when the heterologous DBD binds to its target DNA. The heterologous DBD optionally comprises at its N- and/or C-terminus a peptide linker as described herein. Whether or not a DNA modifying enzyme is active on its target site can be defined by any suitable method known in the art. An exemplary and at the same time preferred method for determining the activity of the DNA modifying enzyme on its target site is as described herein, e.g. by using a plasmid based activity assay (also referred to herein as plasmid-based recombination test) or a PCR-based activity assay (also referred to herein as plasmid-based recombination test) (FIGS. 2A and B). In the plasmid-based assay, the DNA modifying enzyme expression is driven by an (arabinose) inducible promoter in the pEVO vector, carrying the target sites of interest. Upon induction, expression of the DNA modifying enzyme would lead to excision of the DNA fragment between two target sites, resulting in a smaller plasmid size. Plasmid DNA is then extracted from the induced cultures and digested with restriction enzymes, whose sites are flanking the DNA modifying enzyme, the recombined and unrecombined backbones are linearized and size differences can be distinguished on agarose gel. Recombination efficiency is calculated as a ratio between the recombined and unrecombined bands. The PCR-based assay can be used for a quick clonal analysis. To this end, single colonies carrying the pEVO with the DNA modifying enzyme and two target sites are grown in the presence of arabinose overnight. A small amount such as 1 μl of the grown cell suspension is used for a colony three-primer PCR, in which a short elongation time is used that allows generation of only small products. Two bands can then be seen on an agarose gel: a bigger band generated by primer pair P1 and P2 from the unrecombined plasmid, and a smaller band generated by primer pair P1 and P3 from the recombined plasmid.

According to a preferred embodiment, an active DNA modifying enzyme exhibits at least about 5% activity, about 6%, about 7%, about 8%, about 9%, about 10%, preferably at least about 15%, about 20%, about 25%, about 30%, about 35%, about 40%, about 45%, or at least about 50% activity on the respective target site(s). According to a preferred embodiment, DNA modifying enzymes that show less than about 1%, less than about 2%, less than about 3%, less than about 4%, or less than about 5% activity on the respective target site(s) are considered as being inactive on the respective target site(s).

In accordance with a preferred embodiment of the present invention, the DNA modifying enzyme comprises at least two protein monomers, i.e. is in a dimeric form (dimer). Such a dimer can comprise two monomers of the same type (i.e. two identical protein monomers, homodimer) or two monomers of a different type (i.e. two different protein monomers, heterodimer). According to a further preferred embodiment of the present invention, the DNA modifying enzyme comprises at least four protein monomers, i.e. is in a tetrameric form (tetramer). Such a tetramer can comprise four monomers of the same type (i.e. four identical protein monomers, homotetramer), or monomers of a different type such as two, three or four different monomers (heterotetramer). According to a specifically preferred embodiment, the DNA modifying enzyme comprises a heterodimer or heterotetramer comprising different protein monomers, i.e. is a heterodimer or a heterotetramer.

According to a further aspect, the present invention provides a method of changing the specificity and/or activity of a DNA modifying enzyme. The method comprises the steps of (i) identifying an amino acid position of a DNA modifying enzyme for insertion of a heterologous DBD according to the method of the present invention, and (ii) inserting a heterologous DBD at the position identified in step (i). By inserting the DBD at the identified position, the DNA modifying enzyme essentially loses its activity (i.e. becomes essentially inactive) when binding to its target site without the DBD biding to the DBD target site(s). Only if both, the DNA modifying enzyme and the DBD bind to their respective target sites, the DNA modifying activity is essentially restored (i.e. the DNA modifying enzyme becomes essentially active). In that way, a previously unspecific DNA modifying enzyme binding to a plurality of target sites and inducing DNA modifications at various locations in a genome can be tamed to become more specific in that it additionally requires binding of the heterologous DBD to its respective target site(s). This allows modifying DNA modifying enzymes to become active on desired target sites without the necessity of modifying the DNA binding specificity of the enzyme itself. For example, a heterologous DBD highly specific for a certain DNA target site can be inserted into a highly unspecific DNA modifying enzyme, rendering the enzyme highly specific by requiring not only binding of the DNA modifying enzyme to its unspecific target site, but also binding of the inserted DBD to its highly specific target site(s). As shown in Examples 2 and 3, insertional DBD-fusions yield conditional DNA modifying enzymes, and relaxed-type DNA modifying enzymes can be made more specific by insertional fusions with DBDs.

The present invention further provides a nucleic acid or a plurality of nucleic acids encoding the DNA modifying enzyme according to the present invention. According to one embodiment, the nucleic acid sequence or sequences encoding the DNA modifying enzyme of the present invention is/are either already present in a cell or is/are introduced into a cell by conventional means known to the skilled person, such as by recombinant techniques. This may include the step of introducing into the cell said nucleic acid or nucleic acids encoding the DNA modifying enzyme of the invention. According to an alternative embodiment of the present invention, the cell already comprises the nucleic acid or nucleic acids encoding the DNA modifying enzyme of the invention.

The nucleic acid(s) of the present invention may also have the coding sequence fused in frame to a marker sequence, which allows for purification of the DNA modifying enzyme of the present invention. The marker sequence may be an affinity tag or an epitope tag such as a polyhistidine tag, a streptavidin tag, a Xpress tag, a FLAG tag, a cellulose or chitin binding tag, a glutathione-S transferase tag (GST), a hemagglutinin (HA) tag, a c-myc tag or a V5 tag. The HA tag would correspond to an epitope obtained from the influenza hemagglutinin protein, and the c-myc tag may be an epitope from human Myc protein.

The nucleic acid sequence or sequences encoding the DNA modifying enzyme of the invention is/are preferably introduced (e.g. cloned) into a suitable expression vector. Thus, the present invention also provides an expression vector comprising the nucleic acid or plurality of nucleic acids according to the present invention. Introducing the nucleic acid sequence or sequences of the invention into an expression vector can be done by any conventional method known in the art, such as digesting the coding nucleic acid using suitable restriction enzymes and ligating the coding nucleic acid into the expression vector. The expression vector to be used for the library can be any expression vector considered useful by the person of ordinary skill in the art. A preferred expression vector to be used in the context of the present invention is the pEVO expression vector described in Buchholz and Stewart 2001 or a modified version thereof described as pEVO4gg. Further suitable and preferred vectors include pIRES and pEF1a (Lansing et al., 2020, Rojo-Romanos et al., 2023).

For activation of the expression of the nucleic acid or nucleic acids encoding for the DNA modifying enzyme, the nucleic acid or nucleic acids encoding for the DNA modifying enzyme further preferably comprise(s) a regulatory nucleic acid sequence, preferably a promoter region. Hence, expression of the nucleic acid or nucleic acids encoding for the DNA modifying enzyme can be initiated or regulated by activating the regulatory nucleic acid sequence. Accordingly, to induce a DNA modification event, the regulatory nucleic acid sequence (preferably the promoter region) is activated to express the gene encoding for the DNA modifying enzyme. Preferably, the regulatory nucleic acid sequence (preferably the promoter region) is either introduced into a cell, preferably together with the sequence encoding for the DNA modifying enzyme, or the regulatory nucleic acid sequence is already present in a cell. In the second case, merely the nucleic acid encoding for the DNA modifying enzyme must be introduced into the cell (and placed under the control of the regulatory nucleic acid sequence).

The term “regulatory nucleic acid sequence” as used herein refers to gene regulatory regions of DNA. In addition to promoter regions, this term encompasses operator regions more distant from the gene as well as nucleic acid sequences that influence the expression of a gene, such as cis-elements, enhancers or silencers. The term “promoter region” as used herein refers to a nucleotide sequence on the DNA allowing a regulated expression of a gene. The promoter region allows regulated expression of the nucleic acid encoding for the respective protein. The promoter region is located at the 5′-end of the gene and thus before the RNA coding region. Both, bacterial and eukaryotic promoters are applicable for the invention.

The introduction of the nucleic acids into cells is preferably performed using techniques of genetic manipulation known by a person skilled in the art. Among suitable methods are cell transformation, transfection or viral infection, whereby a nucleic acid sequence encoding the protein is introduced into the cell as a component of a vector or part of virus-encoding DNA or RNA. Further preferred methods include delivery as RNA as disclosed e.g. in EP 2590676 Å2 and EP 3115064 Å2, both of which are incorporated herein by reference in their entirety.

Accordingly, the present invention further provides a host cell or culture of host cells comprising the nucleic acid or plurality of nucleic acids according to the present invention, or the expression vector according to the present invention. According to a preferred embodiment, the host cell expresses the DNA modifying enzyme encoded by the nucleic acid or plurality of nucleic acids. According to the present invention, a host cell also includes cellular vesicles derived from such cell and comprising the DNA modifying enzyme of the present invention. A preferred example of a cellular vesicle are exosomes. The present invention can be put into practice using any suitable eukaryotic or prokaryotic cell. Preferred prokaryotic cells are bacterial cells. Specifically preferred prokaryotic cells are cells of Escherichia coli. Preferred eukaryotic cells are yeast cells (preferably Saccharomyces cerevisiae), insect cells, non-insect invertebrate cells, amphibian cells, or mammalian cells (preferably somatic or pluripotent stem cells, including embryonic stem cells and other pluripotent stem cells, like induced pluripotent stem cells, and other native cells or established cell lines, including NIH3T3, CHO, HeLa, HEK293, hiPS). In case of human embryonic stem cells, cells are preferably obtained without destroying human embryos, e.g. by outgrowth of single blastomeres derived from blastocysts, by parthenogenesis, e.g. from a one-pronuclear oocyte, or by parthenogenetic activation of human oocytes. Also preferred are cells of a non-human host organism, preferably non-human germ cells, somatic or pluripotent stem cells, including embryonic stem cells, or blastocytes.

Particularly preferred are isolated host cells that contain a nucleic acid encoding for the DNA modifying enzyme of the present invention, its target site(s), and the target sites for the heterologous DBD.

According to one embodiment of the present invention, the respective recognition or target sites for the DNA modifying enzyme of the present invention and for the heterologous DBD are either included in the cell or introduced into the cell, preferably by recombinant techniques.

Culturing of host cells is preferably carried out by methods known to a person skilled in the art for the culture of the respective cells. Therefore, the host cells of the present invention are preferably transferred into a conventional culture medium, and cultured at temperatures and in a gas atmosphere that is conducive to the survival of the cells and allowing expression of the DNA modifying enzyme of the present invention.

The present invention further provides a pharmaceutical composition comprising the DNA modifying enzyme according to any one of claims 12 to 14, the nucleic acid or plurality of nucleic acids according to the present invention, the expression vector according to the present invention, or the host cell or culture of host cells according to the present invention, and a pharmaceutically acceptable excipient or carrier. The pharmaceutical composition may be in any form that is suitable for the selected mode of administration.

In one embodiment, a pharmaceutical composition of the present invention is administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

The therapeutically active agents as referred to herein include but are not limited to the DNA modifying enzyme of the present invention and may further include the target site(s) of the DNA modifying enzyme of the present invention and of the heterologous DBD. The therapeutically active agents of the invention can be administered, as sole active agent, or in combination with other active agents, in a unit administration form, as a mixture with conventional pharmaceutical supports, to animals and human beings.

In further embodiments, the pharmaceutical composition contains one or more carriers (also termed vehicles) which are pharmaceutically acceptable for a formulation capable of being injected.

These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and storage and must be preserved against the contaminating action of microorganisms, such as bacteria and fungi.

Solutions comprising the therapeutically active agents as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The therapeutically active agents can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be as solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of injectable compositions can be brought about by the use in the compositions of agents delaying absorption, for example, aluminum monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions can be administered in a manner compatible with the dosage formulation and in such amount as is therapeutically effective. The formulations are easily administered in a variety of dosage forms, such as the type of injectable solutions described above, but drug release capsules and the like can also be employed. Multiple doses can also be administered. As appropriate, the therapeutically active agents described herein may be formulated in any suitable vehicle for delivery. For instance, they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but are not limited to water, alcoholic/aqueous solutions, emulsions or suspensions, including saline and buffered media. Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

A physician or veterinarian having ordinary skill in the art may readily determine and prescribe the effective amount of the pharmaceutical composition required. Administration may e.g. be intravenous, intramuscular, intraperitoneal, or subcutaneous, and for instance administered proximal to the site of the target. While it is possible for a delivery system of the present invention to be administered alone, it is preferable to administer the delivery system as a pharmaceutical composition as described above.

Further provided are kits comprising a therapeutically active agent as described herein. In one embodiment, the kit provides the therapeutically active agents prepared in one or more unitary dosage forms ready for administration to a subject, for example in a preloaded syringe or in an ampoule. In another embodiment, the therapeutically active agents are provided in a lyophilized form.

The present invention further provides the use of the DNA modifying enzyme according to the invention, of the nucleic acid or plurality of nucleic acids according to the invention, of the expression vector according to the invention, of the host cell or culture of host cells according to the invention, or the pharmaceutical composition according to the invention, for modifying a nucleic acid sequence of interest. Modifying a nucleic acid sequence of interest can be performed in the genome of at least one isolated or non-isolated cell of a subject. According to one embodiment, the modifying of a nucleic acid sequence of interest in a cell does not include a cell of the human germ line. According to one embodiment, the modification of the nucleic acid takes place in vitro.

The modification of the nucleic acid sequence of interest can be by inserting or exchanging a heterologous nucleic acid sequence, deleting an autologous nucleic acid sequence, or inverting an autologous nucleic acid sequence in said cell or organism. Using the present invention, it is thus possible to induce tissue-specific or site-specific DNA modifications in host organisms, such as mammals, preferably in non-human mammals.

Also provided are host organisms may comprise a vector according to the invention or a nucleic acid according to the invention as described above that is, respectively, stably integrated into the genome of the host organism or of individual cells of the host organism. Preferred host organisms according to the present invention are plants, invertebrates and vertebrates, particularly Bovidae, Drosophila melanogaster, Caenorhabditis elegans, Xenopus laevis, medaka, zebrafish, or Mus musculus, or embryos of these organisms. According to one embodiment, the host organism does not include humans.

According to a further aspect, the present invention provides a method for modifying a nucleic acid sequence of interest, comprising contacting a cell or tissue comprising the nucleic acid sequence of interest with the DNA modifying enzyme according to the invention, the nucleic acid or plurality of nucleic acids according to the invention, the expression vector according to the invention, the host cell or culture of host cells according to the invention, or the pharmaceutical composition according to the invention under conditions allowing the DNA modifying enzyme to modify the nucleic acid sequence of interest. The nucleotide of interest is preferably present in a cell. The cell can be any cell as described herein. According to one embodiment, the method is an in vitro method.

The present disclosure shows that by inserting a DBD into a DNA modifying enzyme, the enzyme's activity and/or specificity may be varied, depending also on the binding of the DBD to its respective target site(s). In order to create respective DNA modifying enzymes for different purposes, a large variety of DBDs with specificity to essentially any DNA target site is needed. To this end, the present invention provides a method of evolving a DBD on a target sequence of interest. The method comprises the steps of:
(i) creating a library of variants of the DBD;
(ii) cloning the library of step (i) into expression vectors comprising a first region encoding a DNA recombining enzyme, a second region comprising a first target site of said DNA recombining enzyme and regions flanking said first target site, and a third region comprising a second target site of said DNA recombining enzyme and regions flanking said second target site, such that a DBD is inserted directly or via peptide linkers into the DNA recombining enzyme, wherein the first, second and third regions are separated from another;
(iii) introducing the expression vectors into host cells and culturing the host cells, thereby expressing the encoded DNA recombining enzyme comprising the DBD;
(iv) isolating plasmids from the cell culture of step (iii) and determining whether the DNA recombining enzyme catalyzed a recombination reaction at both target sites on the vector;
(v) amplifying the DBD of those plasmids that were found to encode a DNA recombining enzyme comprising the DBD and showing recombination activity using error-prone PCR to generate a new library of variants of the DBD;
(vi) repeating steps (ii) to (iv) with the library of step (v).

Regarding the individual steps of the method, respective embodiments and features are described in the present disclosure and in particular herein above and are explicitly incorporated herein. For example, the library of the method of evolving a DBD on a target sequence of interest can be constructed at the DNA level, just like the library according to a preferred embodiment of the method of identifying an amino acid position of a DNA modifying enzyme for insertion of a heterologous DNA binding domain. Thus, according to a preferred embodiment of the present invention, the library of variants of the DBD provided in step (i) of the method is encoded by a nucleic acid library.

The method of evolving a DBD on a target sequence of interest allows the generation of a DBD having a certain specificity for the desired target site. Such a DBD can then be used for being inserted into a DNA modifying enzyme as described herein, thereby rendering the DNA modifying enzyme conditional and specific for the target site of the DBD.

According to a particularly preferred embodiment of the invention, the evolution cycle as defined by steps (i) to (v) of the method of evolving a DBD and in particular a ZF-DBD on a target sequence of interest starts with cloning the DBD library preferably between residues 278 and 279 of a recombinase sequence encoded in the expression vector. The vector carries two lox-zif target sites of interest. After expression of the ZFD-recombinase fusion, plasmid DNA is isolated and analyzed. Upon successful recombination, a unique restriction site between two target sites is excised. By applying restriction digestion, the non-recombined plasmids can be linearized, while the recombined plasmids remain circular. The digestion is followed by a PCR using primers, which generate product only from recombined plasmids. Successful ZF-DBD variants are then used in the next round of evolution by repeating steps (ii) to (iv) as indicated under step (vi). Preferably, counter-selection is applied with vectors containing the lox-sites of interest alone, without the zif motifs.

The present invention also provides a recombinase comprising SEQ ID NO: 41 or an amino acid sequence having at least 90% sequence identity to SEQ ID NO: 41, and SEQ ID NO: 42 or an amino acid sequence having at least 90% sequence identity to SEQ ID NO: 42 for use in treating hemophilia A. According to a preferred embodiment, the recombinase is a dimer. As shown in Example 5, a recombinase comprising SEQ ID NO: 41 or an amino acid sequence having at least 80% sequence identity to SEQ ID NO: 41, and SEQ ID NO: 42 or an amino acid sequence having at least 80% sequence identity to SEQ ID NO: 42 is capable of inverting a genomic locus in the F8 gene. Specifically, said recombinase is capable of correcting an int1h inversion in the F8 gene present in about half of the patients with severe Factor VIII deficiency.

The present invention thus also provides a method for treating hemophilia A comprising administering to the patient a recombinase enzyme comprising SEQ ID NO: 41 or an amino acid sequence having at least 90% sequence identity to SEQ ID NO: 41, and SEQ ID NO: 42 or an amino acid sequence having at least 90% sequence identity to SEQ ID NO: 42 in a therapeutically effective amount.

The present invention also provides a method for treating hemophilia A comprising administering to the patient one or more nucleic acids encoding a recombinase enzyme comprising SEQ ID NO: 41 or an amino acid sequence having at least 90% sequence identity to SEQ ID NO: 41, and SEQ ID NO: 42 or an amino acid sequence having at least 90% sequence identity to SEQ ID NO: 42, in a therapeutically effective amount.

The present invention also provides a method for treating hemophilia A comprising administering to the patient a vector comprising one or more nucleic acids encoding a recombinase enzyme comprising SEQ ID NO: 41 or an amino acid sequence having at least 90% sequence identity to SEQ ID NO: 41, and SEQ ID NO: 42 or an amino acid sequence having at least 90% sequence identity to SEQ ID NO: 42, in a therapeutically effective amount.

The present invention also provides a method for treating hemophilia A comprising administering to the patient a host cell comprising the vector or the one or more nucleic acids of the invention in a therapeutically effective amount.

The present invention also provides a method for treating hemophilia A comprising administering to the patient a pharmaceutical composition comprising a recombinase comprising SEQ ID NO: 41 or an amino acid sequence having at least 90% sequence identity to SEQ ID NO: 41, and SEQ ID NO: 42 or an amino acid sequence having at least 90% sequence identity to SEQ ID NO: 42, the one or more nucleic acids of the invention, the vector of the invention, or the host cell of the invention in a therapeutically effective amount.

The present invention further pertains to the following items.

Item 1: A method of identifying an amino acid position of a DNA modifying enzyme for insertion of a heterologous DNA binding domain (DBD), the method comprising the steps of:
(i) providing a library of DNA modifying enzymes, wherein the members of the library comprise heterologous amino acid sequence insertions throughout the DNA modifying enzyme;
(ii) identifying those DNA modifying enzymes of the library that have DNA modifying activity; and
(iii) identifying the position of the insertion in those DNA modifying enzymes identified in step (ii).

Item 2: The method according to item 1, wherein the one or more positions of the insertions identified in step (iii) are mapped to structural data of the DNA modifying enzyme.

Item 3: The method according to item 1 or 2, wherein the library of DNA modifying enzymes provided in (i) is encoded by a nucleic acid library.

Item 4: The method according to item 3, wherein step (iii) comprises determining at least part of the nucleic acid sequence encoding those DNA modifying enzymes identified in step (ii).

Item 5: The method according to any one of items 1 to 4, comprising the step of selecting one or more amino acid positions for insertion of the heterologous DBD that are surface exposed in the DNA modifying enzyme and in proximity to the DNA binding site of the DNA modifying enzyme.

Item 6: The method according to any one of items 1 to 5, wherein the heterologous amino acid sequence comprised in each of the members of the library of DNA modifying enzymes independent of each other have a length of between three and ten amino acids, preferably a length of five amino acids.

Item 7: A method of producing a DNA modifying enzyme comprising an insertion of a heterologous DBD, the method comprising the steps of:
(i) inserting a nucleic acid sequence encoding the heterologous DBD into a nucleic acid sequence encoding the DNA modifying enzyme at the nucleotide triplet(s) encoding the one or more positions identified in the method of claims 1 to 6; and
(ii) expressing the nucleic acid sequence produced in step (i).

Item 8: The method according to item 7, wherein the nucleic acid sequence encoding the heterologous DBD further comprises a nucleic acid sequence encoding a peptide linker upstream and a peptide linker downstream of the nucleic acid sequence encoding the heterologous DBD.

Item 9: The method according to item 8, wherein the linker is a glycine-serine linker, preferably a glycine-serine linker with at least one G to R substitution.

Item 10: The method according to any one of items 1 to 9, wherein the DBD is a zinc-finger (ZF) DBD or a transcription activator-like effector (TALE) DBD.

Item 11: The method according to any one of items 1 to 10, wherein the DNA modifying enzyme is a transposase or a recombinase, preferably a serine recombinase or a tyrosine recombinase, more preferably a tyrosine recombinase.

Item 12: A DNA modifying enzyme comprising an insertion of a heterologous DBD obtained by a method according to any one of items 7 to 11.

Item 13: A DNA modifying enzyme comprising an insertion of a heterologous DBD, wherein
(i) the DNA modifying enzyme is Cre or a Cre-derived recombinase and the DBD is inserted between amino acid positions 278 and 279; or
(ii) the DNA modifying enzyme is Vika or a Vika-derived recombinase and the DBD is inserted between amino acid positions 172 and 173.

Item 14: A DNA modifying enzyme comprising an insertion of a heterologous DBD, wherein the DBD optionally comprises at its N- and/or C-terminus a peptide linker, wherein the DNA modifying enzyme is inactive on its target site when the heterologous DBD does not bind to its target DNA, and wherein the DNA modifying enzyme is active on its target site when the heterologous DBD binds to its target DNA.

Item 15: A nucleic acid or a plurality of nucleic acids encoding the DNA modifying enzyme according to any one of items 12 or 14.

Item 16: An expression vector comprising the nucleic acid or plurality of nucleic acids according to item 15.

Item 17: A host cell or culture of host cells comprising the nucleic acid or plurality of nucleic acids according to item 15, or the expression vector according to item 16, preferably wherein the host cell expresses the DNA modifying enzyme encoded by the nucleic acid or plurality of nucleic acids.

Item 18: A pharmaceutical composition comprising the DNA modifying enzyme according to any one of items 12 to 14, the nucleic acid or plurality of nucleic acids according to item 15, the expression vector according to item 16, or the host cell or culture of host cells according to item 17, and a pharmaceutically acceptable excipient or carrier.

Item 19: Use of the DNA modifying enzyme according to any one of items 12 to 14, of the nucleic acid or plurality of nucleic acids according to item 15, of the expression vector according to item 16, of the host cell or culture of host cells according to item 17, or of the pharmaceutical composition according to item 18, for modifying a nucleic acid sequence of interest.

Item 20: A method for modifying a nucleic acid sequence of interest, comprising contacting a cell or tissue comprising the nucleic acid sequence of interest with the DNA modifying enzyme according to any one of items 12 to 14, the nucleic acid or plurality of nucleic acids according to item 15, the expression vector according to item 16, the host cell or culture of host cells according to item 17, or the pharmaceutical composition according to item 18 under conditions allowing the DNA modifying enzyme to modify the nucleic acid sequence of interest.

Item 21: Method of changing the specificity and/or activity of a DNA modifying enzyme comprising the steps of:
(i) identifying an amino acid position of a DNA modifying enzyme for insertion of a heterologous DBD according to any one of the item 1 to 6; and
(ii) inserting a heterologous DBD at the position identified in step (i).

Item 22: Method of evolving a DBD on a target sequence of interest, comprising the steps of:
(i) creating a library of variants of the DBD;
(ii) cloning the library of step (i) into expression vectors comprising a first region encoding a DNA recombining enzyme, a second region comprising a first target site of said DNA recombining enzyme and regions flanking said first target site, and a third region comprising a second target site of said DNA recombining enzyme and regions flanking said second target site, such that a DBD is inserted directly or via peptide linkers into the DNA recombining enzyme, wherein the first, second and third regions are separated from another;
(iii) introducing the expression vectors into host cells and culturing the host cells, thereby expressing the encoded DNA recombining enzyme comprising the DBD;
(iv) isolating plasmids from the cell culture of step (iii) and determining whether the DNA recombining enzyme catalyzed a recombination reaction at both target sites on the vector;
(v) amplifying the DBD of those plasmids that were found to encode a DNA recombining enzyme comprising the DBD showing recombination activity using error-prone PCR to generate a new library of variants of the DBD;
(vi) repeating steps (ii) to (iv) with the library of step (v).

### EXAMPLES

### Materials and Methods

### Molecular Cloning

All oligonucleotides were purchased from Sigma-Aldrich. Except for the ZF-SLiDE (described below), all PCR for cloning were performed with high-fidelity Herculase II Phusion DNA polymerase (Agilent). All restriction enzymes were purchased from New England Biolabs. ISOLATE II PCR and Gel Kit (Bioline, Meridian Bioscience) were used for purification of PCR products and DNA fragments isolated from agarose gels. T4 DNA Ligase (Thermo Fisher Scientific) was used for ligation reactions, and 2 μl of the ligation reaction was directly transformed into electrocompetent XL1-blue E. coli bacteria. In case of libraries, the ligation reaction was membrane purified (MF-Millipore) and 4 μl of the ligation was used for transformation. The transformed bacteria were grown overnight in LB medium with addition of antibiotics (30 pg/ml chloramphenicol for pEVO, 100 μg/ml ampicillin for pIRES, pEF1a and pCAGGs plasmids) and with addition of L-arabinose, when induction of recombinase or ZF-recombinase fusions from pEVO were of interest. Plasmids were purified from the overnight cultures using GeneJET Plasmid Miniprep Kit (Thermo Fisher Scientific). Sequence verification was done with Sanger sequencing (Microsynth).

### Plasmids

The plasmid vectors used for tests in bacteria were based on pEVO (described in Buchholz & Stewart 2001; Buchholz & Hauber, 2011; Lansing et al., 2020). The target sites were cloned as described in Lansing et al., 2020. Shortly, primers containing the target sites of interest, BglII restriction site, and an overlap with the pEVO plasmid were designed. These primers were used to produce a PCR product from a pEVO plasmid, which was subsequently cloned into a BglII digested pEVO vector using ColdFusion Cloning Kit (Systems Bioscience). For the target site libraries, each target site was cloned one by one and the plasmids were mixed together in equal ratios. In pEVO, the recombinase or ZF-recombinase fusions were cloned between BsrGI and XbaI or SacI and SbfI restriction sites. Dimer recombinases were cloned between SacI and XhoI (left monomer) and BsrGI and XbaI (right monomer). A Shine-Dalgarno sequence is located in front of each recombinase gene, which in the case of the dimer allowed bicistronic expression of both recombinases. In pEVO, expression of a recombinase or ZF-recombinase fusion complex was induced by arabinose promoter (araBAD). Different L-arabinose (Sigma) concentrations were used for adjusting expression levels of the proteins (from 1 to 200 μg/ml).

Zif268 and ZFCCR5L genes were assembled by using a polymerase cycling assembly method, or for some of the fusions, the sequence of Zif268 was produced by Twist Bioscience. The designed ZFDs were produced by Twist Bioscience.

For the insertional fusion library, first, the XhoI and BsrGI restriction sites were introduced between residues 278 and 279 of Brec1 by overlap extension PCR. Subsequently, the linker library was created by performing PCR with a mix of primers containing different numbers (from 1 to 8) of GGS repeats, overlapping with Zif268 and XhoI (for the forward primers) or BsrGI (for the reverse primers) restriction sites. Digested PCR product of Zif268 flanked by the linker libraries was subsequently cloned into pEVO_Brec1 vector between residues 278 and 279 via XhoI and BsrGI restriction sites. During the cloning of libraries, a coverage of at least 100,000 clones was reached.

For cloning of single ZF-recombinase fusion complexes, either the same cloning strategy as described for the libraries was employed, or BbvCI and PspOMI restriction sites were introduced between the residues 278 and 279 of the recombinase cloned into pEVO between BsrGI and XbaI restriction sites using overlap extension PCR. The primers with the overhangs containing the BbvCI and PspOMI restriction sites and the (GGS)₈ linker sequences were used for amplifying the Zif268 or designed ZFPs, and digested PCR product was cloned into the recombinase sequence. The construct RecFlex278-Zif268 was produced by Twist Bioscience.

For transient expression of Brec1 or Brec1-Zif268 in HEK293T cells, these genes were cloned via BsrGI and XbaI restriction sites into the pIRES-NLS-EGFP vector (Lansing et al., 2020) (FIG. 1B). For transient expression of D7 or D7-ZF heterodimer in HEK293T cells, the monomers were cloned via BsrGI and XbaI restriction sites into a mammalian expression vector (pEF1a-mTagBFP-P2 Å-NLS-RecL or pEF1a-EGFP-P2 Å-NLS-RecR) (FIG. 1C). In this vector, the recombinase or ZF-recombinase complex was translationally linked with mTagBFP or EGFP using a P2A self-cleaving peptide sequence. Expression of this construct was driven by EF1a promoter.

The pCAGGs-lox-pA-lox-mCherry reporter plasmid was generated as described in Lansing et al., 2020, in Lansing et al., 2022, and in Rojo-Romanos et al., 2023 (FIG. 1A). In short, the loxBTR, loxBTR-5-zif (A) and loxBTR-5-zif (B) target sites were introduced by PCR with overhang primers and were cloned into the pCAGGs vector via SalI and EcoRI restriction sites.

### Pentapeptide Scanning Mutagenesis

Pentapeptide scanning mutagenesis was done using the Mutation Generation System Kit (Thermo Fisher Scientific), according to manufacturer's instructions. In short, the M1-KanR Entranceposon was inserted into the pEVO containing the recombinase using in vitro Mu transposition reaction. To select for the variants where the transposon was inserted within the recombinase sequence, plasmid DNA of the obtained libraries was digested with BsrGI and XbaI restriction enzymes, and a DNA fragment that indicated successful integration of the transposon into the recombinase sequence (around 2 kb) was extracted and subcloned into a fresh pEVO vector. Next, the Entranceposon was removed from the library by NotI restriction digestion, and the mutated library containing five amino acid in-frame insertions throughout the recombinase sequence was cloned into the pEVO vectors containing recombinases respective lox-sites. Expression was induced by arabinose supplement to the medium. At this step, to confirm randomness of the mutations, single clones were sequenced and analyzed for recombination activity by PCR (described herein as “PCR for assessing recombination activity”). To select only the mutated recombinase variants which retained recombination activity, 500 ng of the induced library plasmid DNA was digested with NdeI and AvrII, that are located between the two lox-sites on the pEVO plasmid. Thereby, the variants that did not excise the DNA sequence between the two lox-sites were digested and removed from the pool, while the plasmids carrying the active variants remained intact. The digested library was then membrane purified, transformed and grown overnight with arabinose supplement. The next day, 500 ng of the active library DNA was again digested with NdeI and AvrII and 25 ng of the digested DNA was used as a template for high-fidelity PCR to amplify the active mutated recombinases, which were digested with BsrGI and XbaI and subcloned to a fresh pEVO vector containing the respective lox-sites and induced with arabinose. The selection cycle was repeated twice. At the final step, the plasmid DNA of active mutated recombinase libraries was extracted and prepared for the long-read PacBio sequencing (described herein as “deep sequencing”).

### Deep Sequencing

Long-read PacBio sequencing of the active libraries of the mutated Cre, Brec1, D7L, D7R after the pentapeptide scanning mutagenesis was performed as previously described by Schmitt et al., 2022.

Nanopore sequencing of the active libraries of the mutated Vika, Vika2, Vika3, Bxb1, SH1_c129, SH2_c121, SH2_c126, SH3_c1326, and SH4_c1779 recombinases after the pentapeptide scanning mutagenesis was performed in the following way: The plasmid DNA of the obtained libraries of the active mutants of Vika, Vika-like recombinases, Bxb1 and Bxb1-derived recombinases was extracted and fragments containing the recombinases and target sites were obtained by digesting with BsrGI and ScaI restriction enzymes and subsequent gel extraction. The library preparation was performed following the protocol “Native barcoding amplicons” using the SQK-LSK110 and the EXP-NBD104 kit (Oxford Nanopore Technologies). The three libraries were mixed before the preparation in a 1:1:1 ratio. Sequencing was performed on MinION Mk1B Nanopore sequencer with the FLO-MIN106 r9.4.1 or FLO-MIN106 r0.4.1 flowcell (Oxford Nanopore Technologies).

The high-throughput screen for testing different combinations of linkers and spacing lengths to develop the ZF-recombinase fusion architecture was performed as follows: The libraries of Brec1-Zif268 fusions were cloned to the pEVO target site library, transformed into XL1-blue E. coli and grown for 14-16 h in LB supplemented with chloramphenicol. Brec1-Zif268 expression was induced by 200 μg/ml L-arabinose. The plasmid DNA was extracted and fragments containing Brec1-Zif268 fusion complexes and target sites were obtained by digesting with SacI and ScaI restriction enzymes and subsequent gel extraction. The library preparation was performed following the protocol “Native barcoding amplicons” using the SQK-LSK110 and the EXP-NBD104 kit (Oxford Nanopore Technologies). Sequencing was performed on a MinION Mk1B Nanopore sequencer with the FLO-MIN106 r9.4.1 flowcell (Oxford Nanopore Technologies).

For an analysis, PacBio HiFi DNA sequences after the pentapeptide scanning mutagenesis screen for Cre-type recombinases were aligned to the wild-type DNA reference sequence (Brec1, Cre, D7L or D7R) using exonerate (version 2.3.0) with the “affine:bestfit” model. From this alignment, the CIGAR values for each read were processed with a custom R script that counts 15 bp insertions for each position (R version 4.1.1 with tidyverse packages version 1.3.164). All nanopore sequencing data was base-called with guppy (Oxford Nanopore Technologies, version 5.0.7) in high accuracy mode. Only Reads with a Phred quality score of 10 or more were retained for further processing.

Sequencing reads from the pentapeptide scanning mutagenesis screen for Vika-type recombinases were aligned in two phases. In the first demultiplexing phase, all reads were aligned to sequences of backbones containing Vika (SEQ ID NO: 8), Vika2 (SEQ ID NO: 14) and Vika3 (SEQ ID NO: 15) and their respective target sites in unrecombined and recombined variants—six reference sequences in total. Subsets of reads unambiguously mapping to each of the references were individually subjected to the second alignment phase, in which each subset was mapped to a library of corresponding recombinase sequences containing pentapetide insertions at each possible position. The final recombination rates of each protein variant were obtained by calculating a fraction of counts of reads mapped to a recombinase variant with recombined target sites, to counts of all reads mapped the recombinase variant with either recombined or unrecombined target sites.

Sequencing reads from the high-throughput screen for testing different combinations of linkers and spacing lengths were aligned to all possible sequence combinations (ZF fusion type, linker length, spacing between the loxBTR and zif268 target sites, recombined or non-recombined target sites) using minimap2 (version 2.17,65) with the “secondary” option set to “no”. The alignment file was then filtered with samtools (version 1.1166) using the view command and the −L option to only include reads that cover the target site and the recombinase by supplying bed files that contain specific coordinates for these regions. Relevant information from this alignment file was then extracted using GNU Awk (version 5.1.1) and processed and visualized in R (version 4.1.1 with tidyverse packages version 1.3.164). The recombination rates were calculated by counting the number of recombined and non-recombined reads for each ZF-recombinase complex and target site combination.

### PCR for Assessing Recombination Activity

For a quick clonal analysis, the recombination activity was assessed by a PCR-based assay, described in Lansing et al. 2020 and Lansing et al., 2022. In short, after transformation, the recovery was plated on agar with chloramphenicol (15 μg/ml). Single colonies were picked and grown in 500 μl of LB in the presence of chloramphenicol and L-arabinose in a 96 deep-well plate for 16 h. One microliter of the grown cell suspension was used for colony three-primer PCR with MyTaq Polymerase (Bioline) (FIG. 2A). A first primer binds between two lox-sites, and a second primer binds upstream of the lox-sites. Therefore, this primer combination generates a PCR product of approximately 500 bp, indicating the non-recombined substrate. A third primer binds downstream the second lox-site and a combination with the second primer will generate a shorter approximately 400 bp product, indicating the recombined plasmid.

### Plasmid-Based Recombination Test in Bacteria

To assess recombination activity, the efficiency of the excision of the two target sites on the pEVO plasmid was evaluated. To this end, the expression of the recombinase or ZF-recombinase fusion in overnight cultures was induced by addition of L-arabinose. Testing of the fused ZF-recombinase complexes and recombinases alone on the same target sites was performed at the same concentration of L-arabinose, but the induction levels varied between the different experiments, depending on the activity of the non-fused recombinase on the respective target sites (200 μg/ml were used for Brec1-loxBTR, D7R-loxF8R, RecFlex-loxMECP2 and SH2c121-T285; 100 μg/ml for RecHTLV-loxHTLV, D7L-loxF8L, D7-loxF8 and all off-targets, RecFlex-loxFlex1, RecFlex-loxFlex4, Vika2-vox2, Vika3-vox3, Vika4-vox4; 10 μg/ml for RecHTLV, RecFlex-loxHex2, RecFlex-loxFlex3, RecFlex-loxFlex5, and Bxb1-T285; 7 μg/ml for SH2c121-D478; 5 μg/ml for SH2c121-R467; 1 μg/ml for Bxb1-R467; 0 μg/ml for SH2c121-G489). 500 ng of plasmid DNA extracted from induced cultures was digested with BsrGI and XbaI or SacI and SbfI restriction enzymes. 200 ng of the digested DNA was loaded onto a 0.8% agarose gel stained with RedSafe (Intron Biotechnology) and the gel was run at 70 V for 90 min. Three bands could be seen on the agarose gel after gel electrophoresis. The smallest band of 1 kb shows the recombinase (approximately 1.5 kb for the recombinase fused with a ZFD, approximately 2 kb for the recombinase heterodimer (D7), approximately 3 kb for the recombinase heterodimer fused with ZFP (D7-ZF)), and was used as a control for the presence of the tested recombinase or recombinase fusion in the digested plasmid pool. The biggest band of approximately 5 kb shows the unrecombined pEVO backbone, and a smaller band of approximately 4.3 kb shows the recombined substrate. Gel images were taken with Infinity VX2-3026 transilluminator, using the Infinity Capt software (Vilber). Band intensities (bands at 5 kb and 4.3 kb) were calculated using Fiji (Version 2.0.0.-rc-65/1.52a). Recombination efficiency was quantified by the ratio of non-recombined to recombined band intensities. A scheme of the test is shown in FIG. 2B.

### Sequence Analysis of the Evolved ZFDs

Active clones from the final ZFL (75 clones) and ZFR (59 clones) libraries were picked and sent for E. coli overnight Sanger sequencing (Microsynth). The obtained sequences were analyzed to determine the mutational changes in the ZFD and linker sequence by comparing to the respective ZFL1 and ZFR4 sequences. Analysis of the sequencing data was performed in R v4.1.0 using dplyr sequence tools (https://github.com/ltschmitt/SequenceTools) and ggplot2 packages.

### Cell Cultures

The HEK293T (ATCC) cells were cultured in Dulbecco's modified Eagle's medium (DMEM, Gibco) with 10% fetal bovine serum (Gibco) and 1% Penicillin-Streptomycin (10,000 U/ml, ThermoFisher) at 37° C., 5% CO₂ in HERAcell Incubator 240i (ThermoFischer Scientific). Patient-derived F8 hiPSCs were reprogrammed at the Stem Cell Engineering Facility of the Center for Molecular and Cellular Bioengineering (CMCB) at the TU Dresden (described in Lansing et al., 2022). hiPSCs were cultured in StemFit Basic04 Complete Type (AJINOMOTO), the first 24 h after splitting the medium was supplemented with 10 μM Rock-inhibitor (Y-27632, Tocris). Accutase (TheromFisher) was used for detachment of the cells for splitting. The coating was performed with iMatrix-511 silk laminin (NIPPI) according to manufacturer's instructions.

### Cell Culture Plasmid Recombination Assay

To test the activity of Brec-Zif268 fusion complexes, a plasmid assay in HEK293T cells was performed. 30,000 HEK293T cells/well were seeded in a 96 well plate overnight. On the following day, 25 ng of the pIRES expression plasmid and 25 ng of the pCAGGs reporter plasmid were transfected using Lipofectamine 2000 Transfection Reagent (ThermoFisher). Cells in each well were analyzed with a MacsQuant VYB (Miltenyi) 48 hours after transfection. HEK293T cells were gated for single cells, for transfected population (GFP⁺ cells), and finally for transfected cells that successfully performed the recombination of the reporter (mCherry*GFP⁺ cells). Recombination efficiency was calculated by the percentage of double positive cells (mCherry*GFP⁺) divided by the percentage of all GFP⁺ cells. Analysis of the data was performed using FlowJo™ 10 (BD).

To test the inversion efficiency of the genomic loxF8 locus, HEK293T cells were transfected with pEF1a expression plasmids expressing D7 or D7-ZF. For this, 200,000 HEK293T cells/well were seeded in a 12 well plate. On the next day 400 ng of pEF1a plasmid expressing D7L or D7L-ZFL and 400 ng of pEF1a plasmid expressing D7R or D7R-ZFR were transfected using Lipofectamine 2000 Transfection Reagent (ThermoFisher). 72 hours after transfection the cells were analysed with a MacsQuant VYB (Miltenyi) and harvested. To determine transfection efficiency, HEK293T cells were gated for single cells and for transfected population (GFP+BFP+ cells). In both experiments, analysis of the flow cytometry data was performed using FlowJo™ 10 (BD).

### In Vitro Transcription

DNA templates for in vitro transcription (IVT) were generated by PCR from the pEF1a plasmids with EGFP, D7L or D7L-ZFL, D7R or D7R-ZFR. D7L, D7R, D7L-ZFL(G10), D7R-ZFR(G10) and eGFP mRNA was produced using the HiScribe™ T7 ARCA mRNA Kit (NEB) and purified using the Monarch© RNA Cleanup Kit (NEB), according to manufacturer's instructions.

mRNA Transfection

Patient-derived F8 hiPSCs were transfected with IVT produced mRNA using Lipofectamine™ MessengerMAX™ Transfection Reagent (ThermoFisher). F8 hiPSCs were seeded at a density of 600,000 cells/well in a 6-well format the day before transfection. For each well 740 fmol of recombinase mRNA (250 ng D7L and D7R mRNA, or 360 ng D7L-ZFL mRNA and 380 ng D7R-ZFR mRNA), 50 ng eGFP mRNA was used for transfection. Cells were analyzed 48 h post transfection by fluorescent microscopy and harvested.

Detection and quantification of the int1h inversion by PCR on genomic DNA Genomic DNA from HEK293T and F8 hiPSCs cells transfected with D7 or D7-ZF was isolated using the QIAamp DNA Blood Mini Kit (Qiagen). The inversion of the 140 kb DNA fragment between the two loxF8 target sites was detected by PCR, as described previously by Lansing et al., 2022.

Inversion efficiency was quantified using a qPCR-based assay as described previously by Lansing et al., 2022. A TaqMan amplicon specific probe was used. Samples of 1%, 5%, 10%, 25%, 50%, and 100% inversion were generated by mixing genomic DNA of WT iPSCs and F8 hiPSCs at appropriate ratios. The Cq values of these mixtures were used to build a standard curve and extrapolate the inversion efficiency of the genomic DNA samples of interest. The calculated inversion efficiencies from the transfected HEK293T cells were normalized by transfection efficiencies. Since genomic DNA of male iPSCs (one X-chromosome) was used for generation of the standard curve used in the quantification, the calculated inversion efficiencies from the transfected HEK293T cells (female, two X-chromosomes) were divided by two. For quantification in F8 hiPSCs, an average of the triplicate samples transfected with D7 was calculated and fold-change of each replicate treated with D7-ZF was quantified using the formula: (D7-ZF inversion−D7 inversion average)/D7 inversion average.

### Off-Target Analysis

Position Weight Matrices (PWM) predicting the recognition motif for the evolved zinc finger domains ZFL and ZFR in the selected D7-ZF clone were obtained using the Interactive PWM Predictor (Persikov & Singh, 2014). Potential genomic off-targets of D7 recombinases (Lansing et al., 2022) were scanned for occurrences of the PWM motifs using FIMO tool from the MEME Suite (Grant et al., 2011), using a p-value threshold of 0.001. Reported results were filtered to ensure that coordinates of matches are within an expected distance of 4 bp to 6 bp from the corresponding “left” or “right” half-site.

### Example 1: Determination of Potential Sites for Insertional ZFD-Fusions

To identify positions within Cre-derived DNA modifying enzymes that can tolerate small insertions, pentapeptide scanning mutagenesis was performed as described in Hayes & Hallet, 2000, Petyuk et al., 2004, and as detailed above. Four different recombinases were used, namely Cre, Brec (SEQ ID NO: 4) (described in Karpinski et al., 2016), D7L (SEQ ID NO: 17) and D7R (SEQ ID NO: 18) (both described in Lansing et al., 2022). Using in vitro Mu transposition (Haapa et al., 1999), four libraries with insertions of five amino acids were created. Enzyme variants that retained recombinase activity were selected, followed by long-read sequencing.

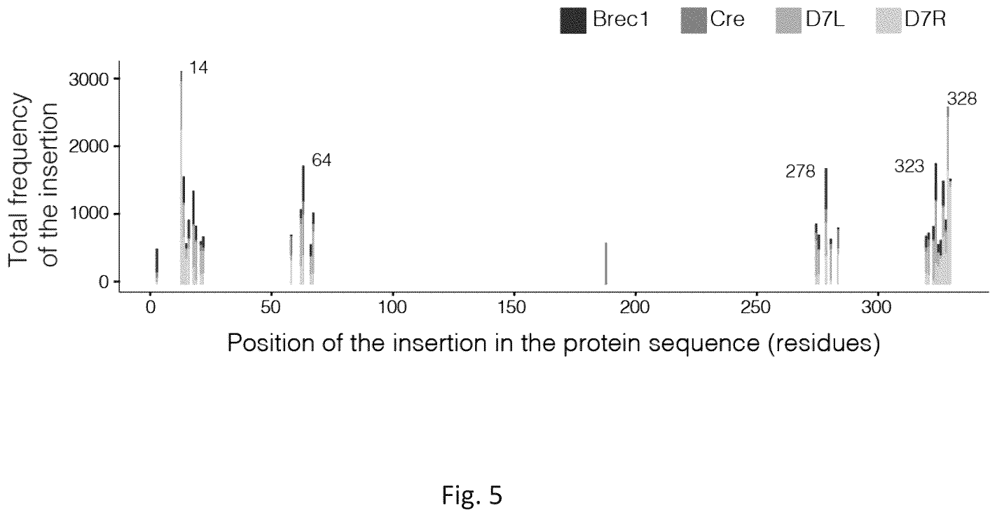

Mapping the reads revealed that insertions in Cre were tolerated at numerous positions, reflecting the robustness of the enzyme for insertional mutagenesis (Petyuk et al., 2004) (FIG. 4). In contrast, the designer-recombinases did not exhibit the same pattern, with fewer regions allowing insertions of the five amino acids. However, the active mutants of Cre, Brec1, D7L, and D7R contained insertions in identical regions of the proteins, suggesting these areas as potential universal insertion sites (FIG. 5).

To identify a universal position in the recombinase sequence for insertional fusions in Cre and in Cre-type recombinases, the most frequent positions that were found to be tolerated in the pentapeptide mutagenesis screen in all four recombinases were considered: aa14, aa64, aa278, aa323, and aa328. The crystal structure of the Cre/loxP synapse pre-cleavage complex (PDB ID 1Q3U (Ennifar, 2003)) was inspected to nominate the best position for the ZF insertion using the 3D Protein imager (Tomasello et al., 2020) for the analysis. The available experimental structures of Cre lack information about its N-terminal tail (aa 1-20), therefore, position aa14 was excluded from the analysis. Visual inspection of the selected positions revealed that residues N323 and L328 are positioned in an area of extensive protein-protein interaction of the C-terminal domains of the monomers, while residues F64 and D278 are located on the exposed surface of the dimer complex and are not involved in protein-protein or protein-DNA interactions (FIG. 6A). Next, the distance from these residues to the nucleotide following the loxP target site was estimated, which amounted to 52.2 Angstroms for F64 and 30.4 Angstroms for D278 (FIG. 6B). According to the spatial accessibility and closer proximity to the DNA, the position in the recombinase sequence between residues 278 and 279 was selected for the insertional fusion of a zinc finger protein.

To identify a universal position in the recombinase sequence for insertional fusions in Vika-type recombinases, the same approach using pentapeptide scanning mutagenesis was applied. A 3D model of wt Vika (SEQ ID NO: 8) using the online tool ColabFold was generated, which predicts protein structure using AlphaFold2 and Alphafold2-multimer. Protein alignments and templates were performed using MMseqs2 and HHsearch (Mirdita et al., 2022). The generated model was imported in 3D Protein Imager (Tomasello et al., 2020) and superimposed to the crystal structure of the Cre/loxP synapse pre-cleavage complex (PDB ID 1Q3U (Ennifar, 2003)) for visual inspection of the selected positions and calculation of the distance from these residues to the nucleotide following the loxP target site. The shortest distance was observed for the residue S172 which amounted to 17.60 Angstroms (FIG. 7). Insertion between positions S172 and V173 of ZFD was chosen and recombination activity tests on vox- and vox-like wt and vox-zif and vox-like-zif target sites were performed (FIG. 12).

To identify the positions within Bxb1 and Bxb1 evolved clones that can tolerate small insertions, pentapeptide scanning mutagenesis using in vitro Mu transposition was performed as described above. Six different recombinases were used, namely Bxb1, SH1 c129, SH2 c121, SH2 c126, SH3 c1326, SH4 c1779. The libraries with insertions of five amino acids were created and expressed on the respective recombinase target sites: Bxb1 library—on Batt-B_Batt-P52, SH1 c129 library—on Batt-SH1-B_Batt-SH1-P, SH2 c121 and SH2 c126 libraries—on Batt-SH2-B_Batt-SH2-P, SH3 c1326 library—on Batt-SH3-B_Batt-SH3-P, SH4 c1779 library—on Batt-SH4-B_Batt-SH4-P. Upon recombinase expression, the libraries were subjected to nanopore sequencing over the recombinase and target site sequence. The results of the sequencing were used for calculating recombination efficiency of the variants on their target sites through the ratio of the recombined vs unrecombined reads. The number of the reads from the variants that retained at least 10% recombination activity were used for mapping.

Mapping the reads revealed that similar to Cre, insertions in Bxb1 were tolerated in numerous positions, reflecting the robustness of the enzyme for insertional mutagenesis. In contrast, the designer-recombinases showed fewer regions allowing insertions of the five amino acids. Nevertheless, the active mutants of Bxb1, SH1 c129, SH2 c121, SH2 c126, SH3 c1326, SH4 c1779 contained insertions in identical regions of the proteins, suggesting these areas as potential universal insertion sites (FIG. 24).

To identify a universal position in the recombinase sequence for insertional ZFD-fusions in LSRs, the wt Bxb1 and Bxb1-type recombinases, the most frequent positions were found to be in the pentapeptide scanning mutagenesis screen in all six recombinases were considered: aa285, aa467, aa478, aa489 (FIG. 25). A 3D model of Bxb1 using the online tool ColabFold was generated, which predicts protein structure using AlphaFold2 and Alphafold2-multimer. The generated model was imported in 3D Protein Imager (Tomasello et al., 2020) and superimposed to the C-terminal domains of a serine integrase A118 bound to its attP DNA half-site (PDB ID 4KIS (Rutherford, 2013)). The most frequent positions that tolerated the insertions in the sequence of Bxb1 and five Bxb1-derived evolved recombinases were inspected on the 3D protein model of Bxb1. The distance from these residues to the nucleotide following the attP target site was estimated, which amounted to 42.84 Angstroms for T285, 9.36 Angstroms for R467, 38.24 Angstroms for D478, and 48.44 Angstroms for G489 (FIG. 26). Since all four positions are in close proximity to DNA with less than 50 Angstroms, all of them were selected for the insertional fusion of a zinc finger protein.

### Example 2: Insertional ZFD-Fusions Yield Conditional Recombinases

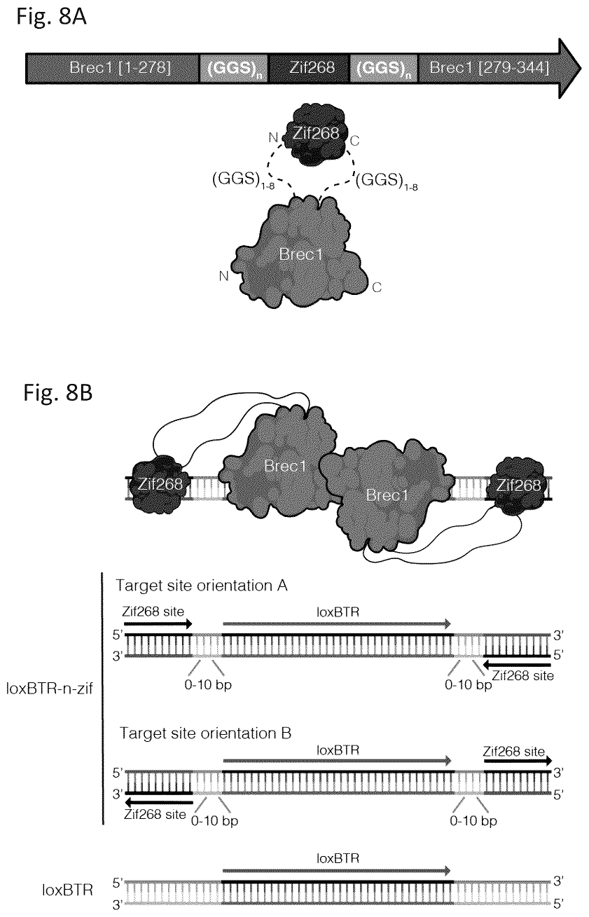

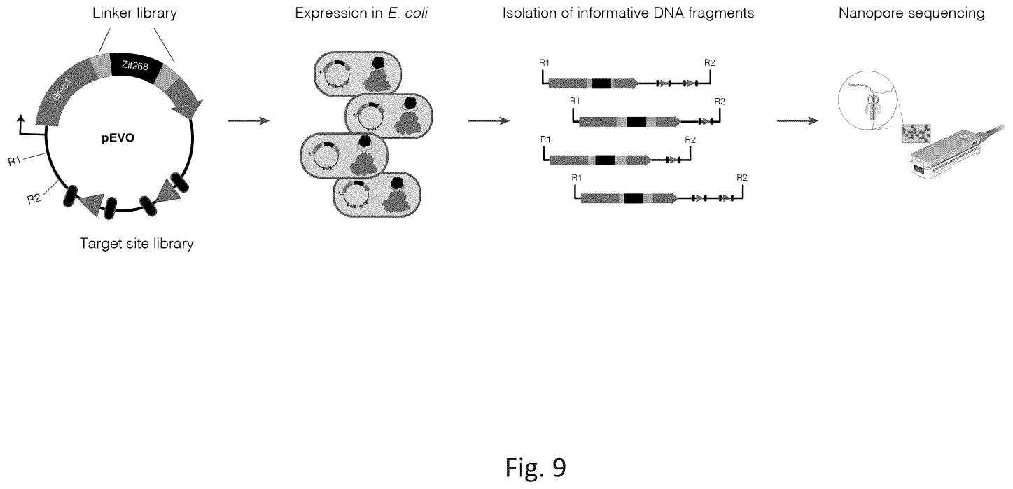

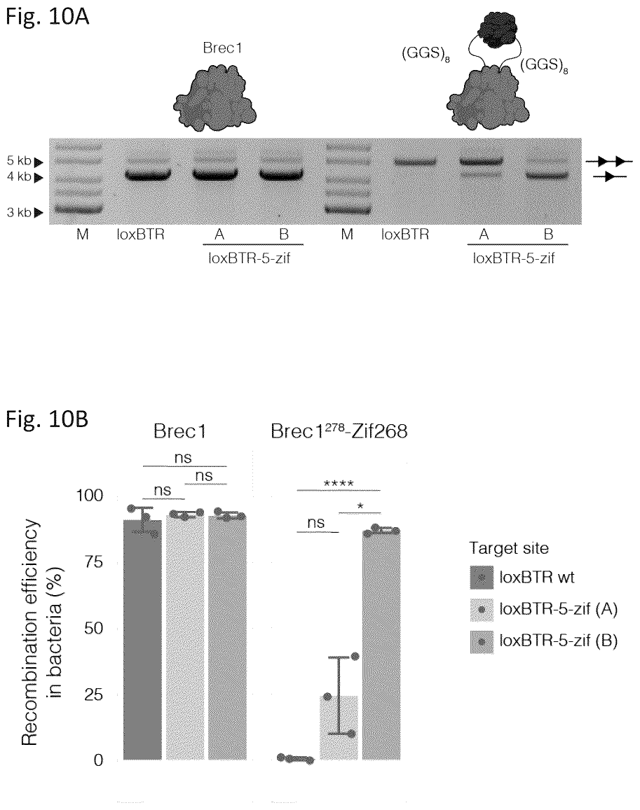

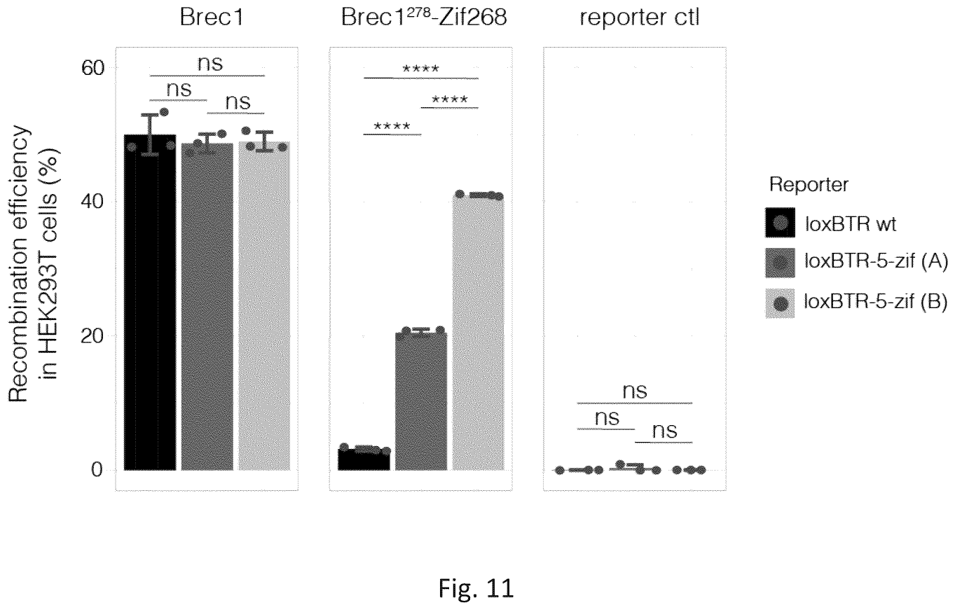

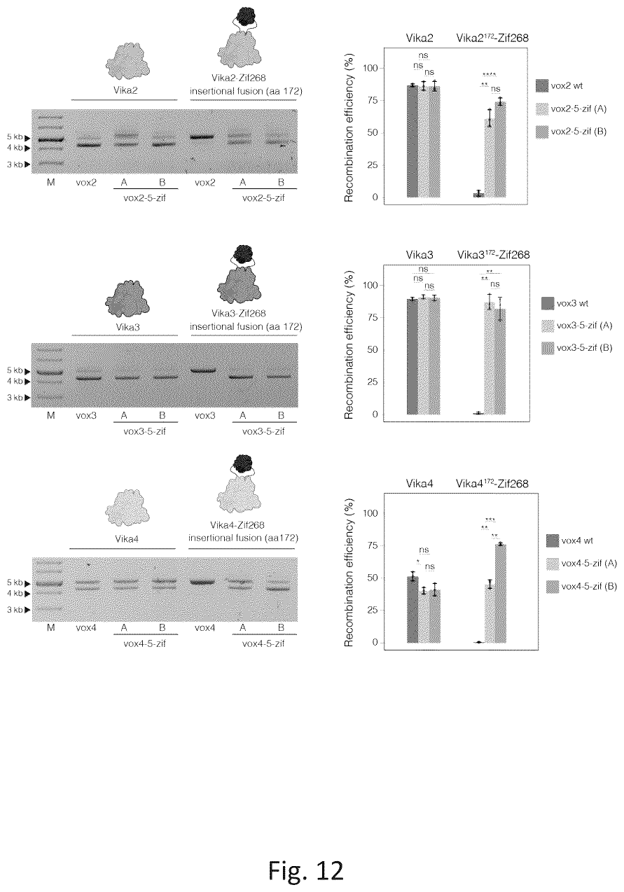

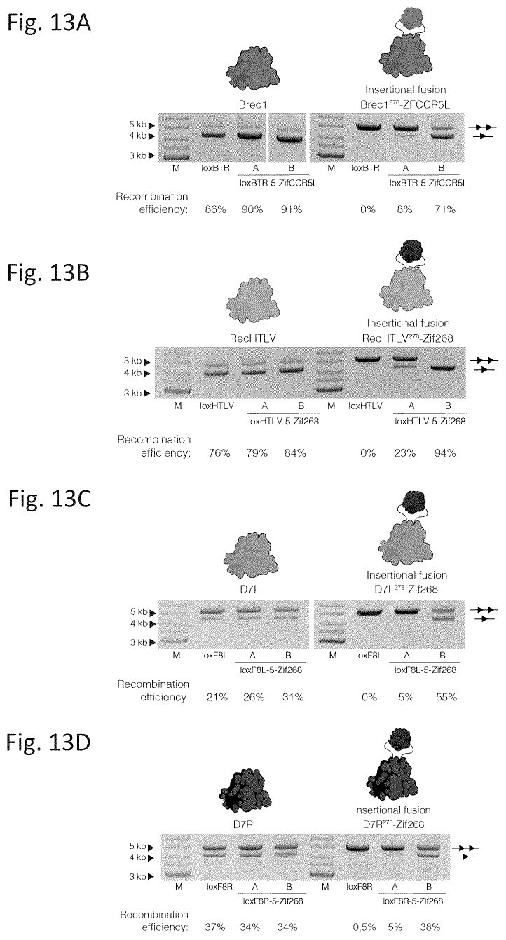

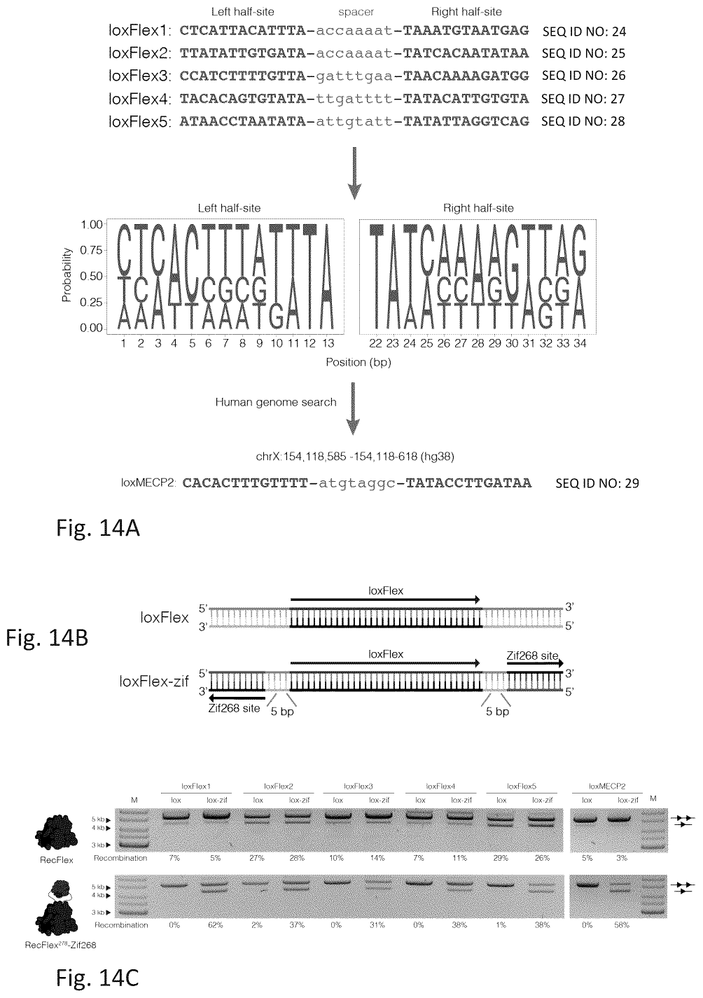

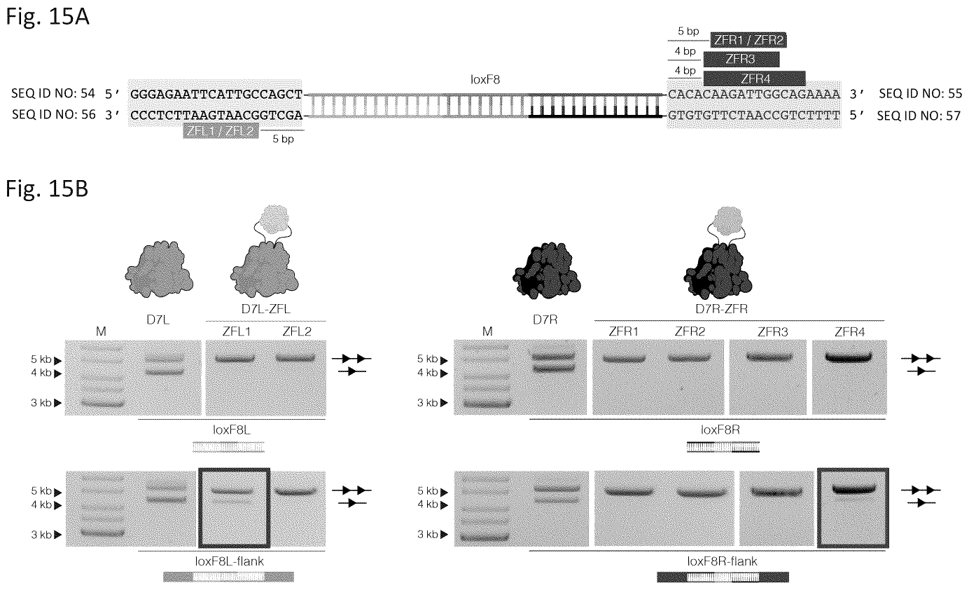

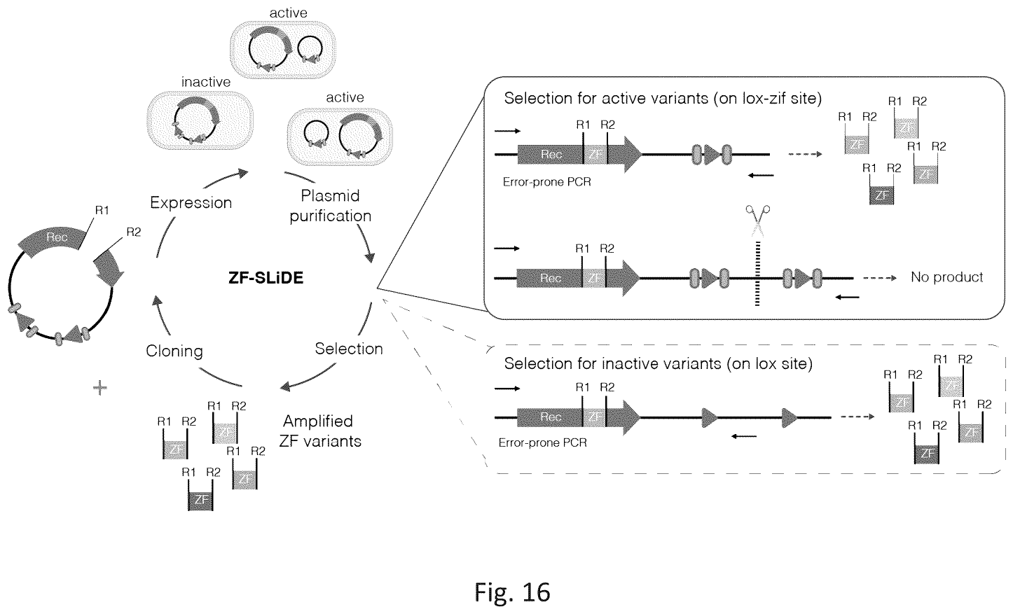

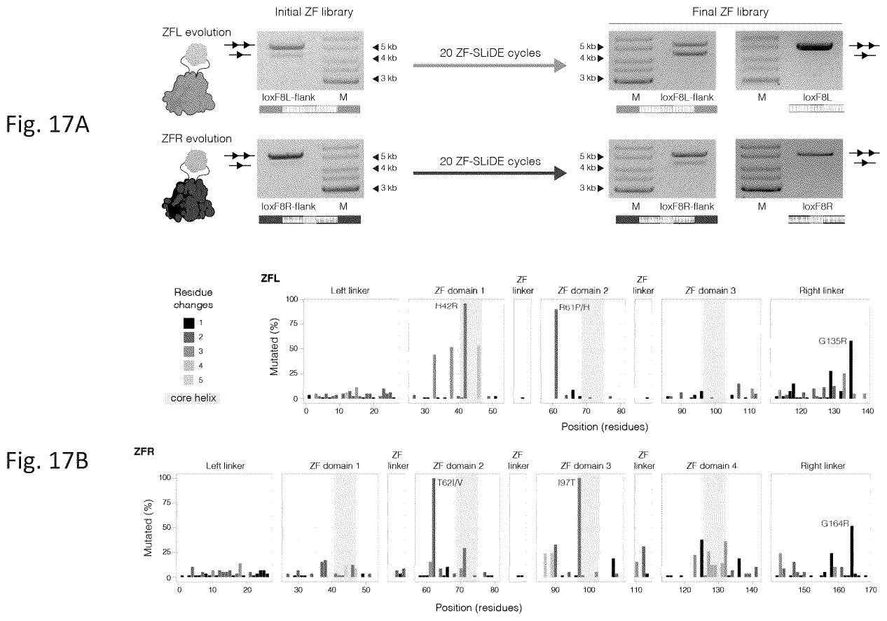

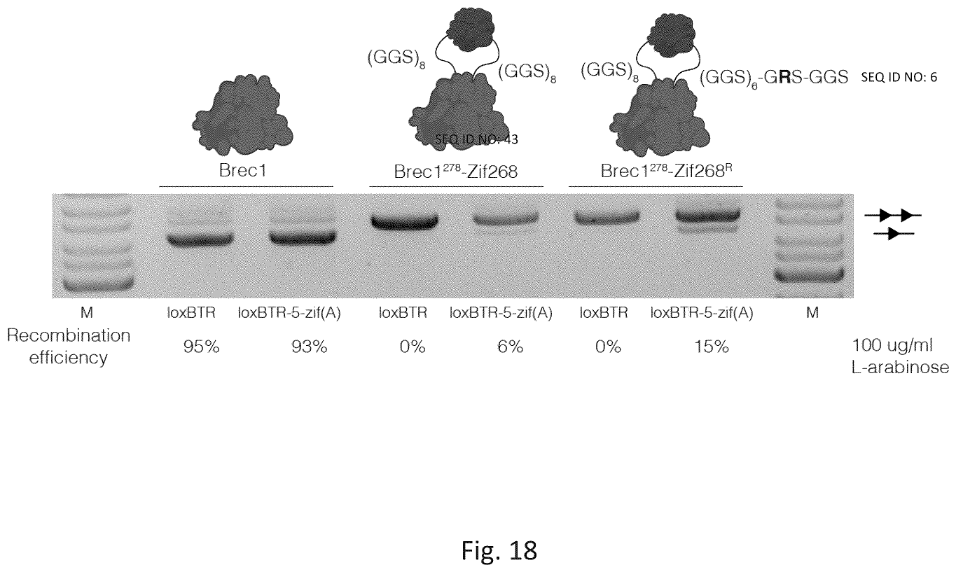

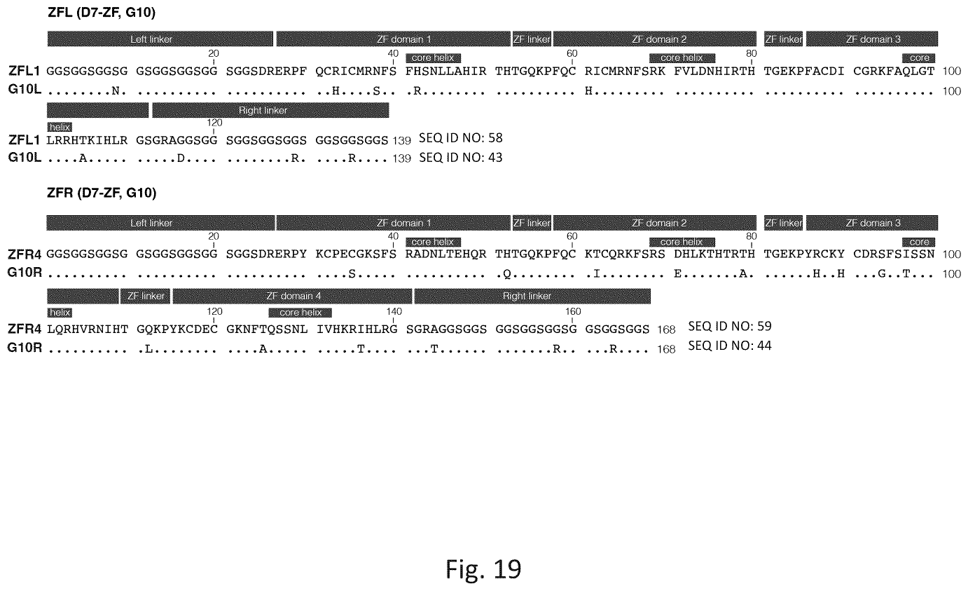

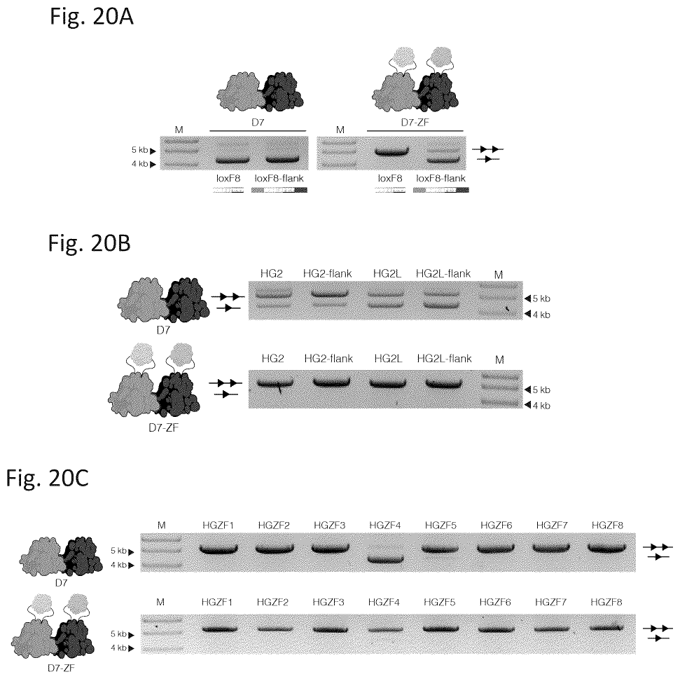

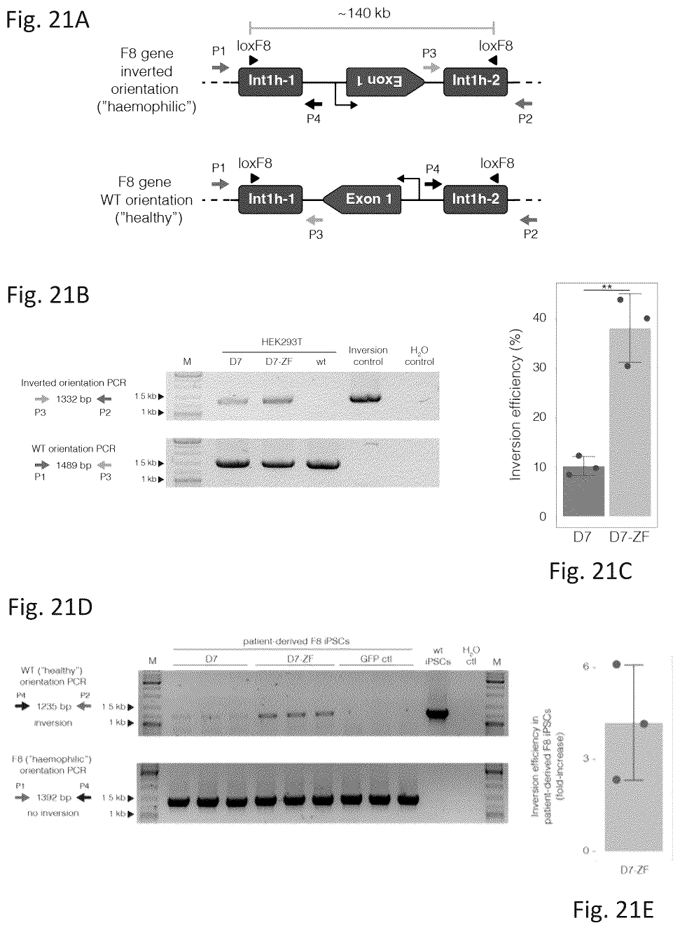

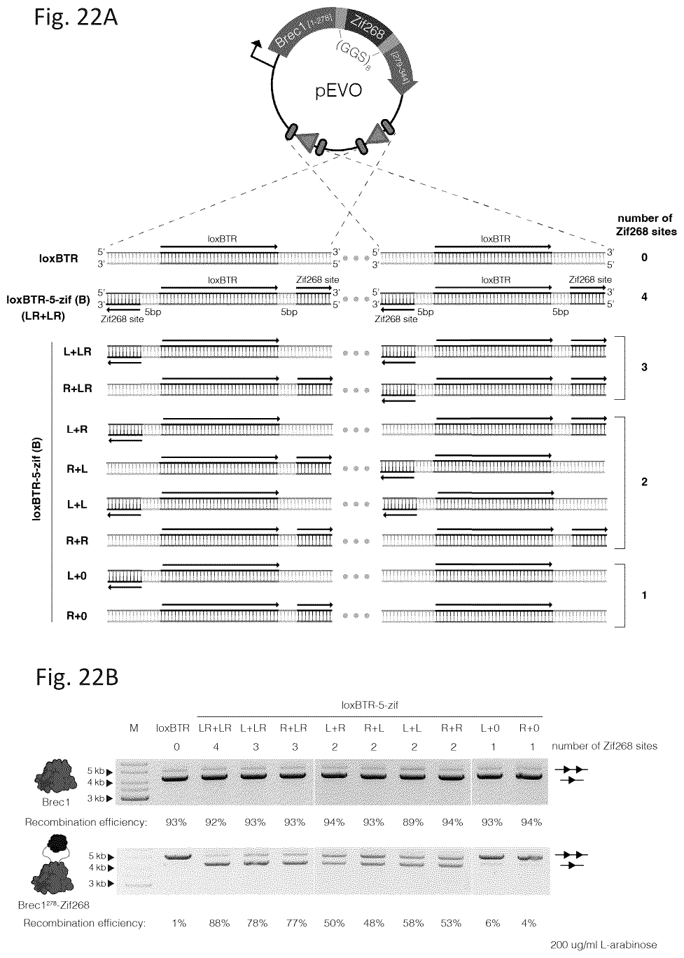

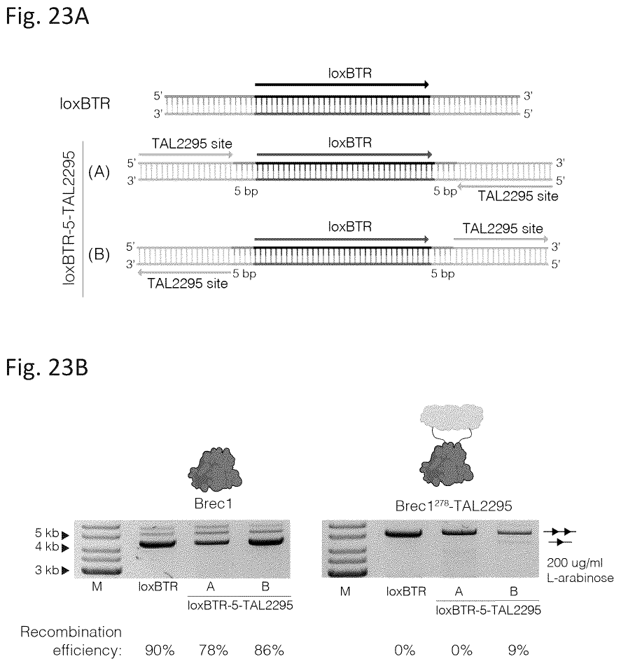

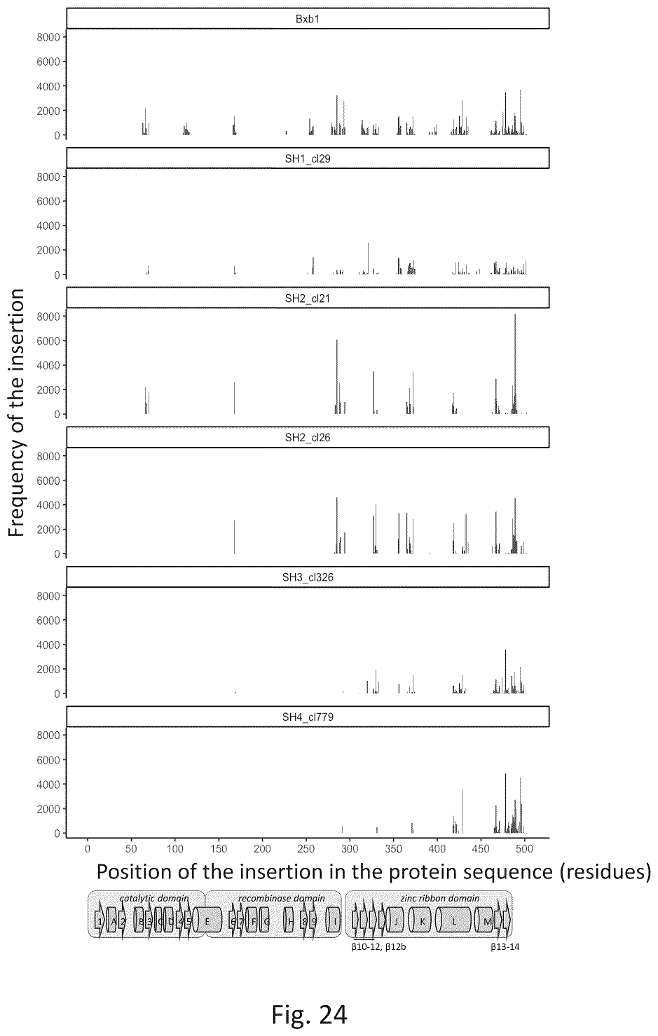

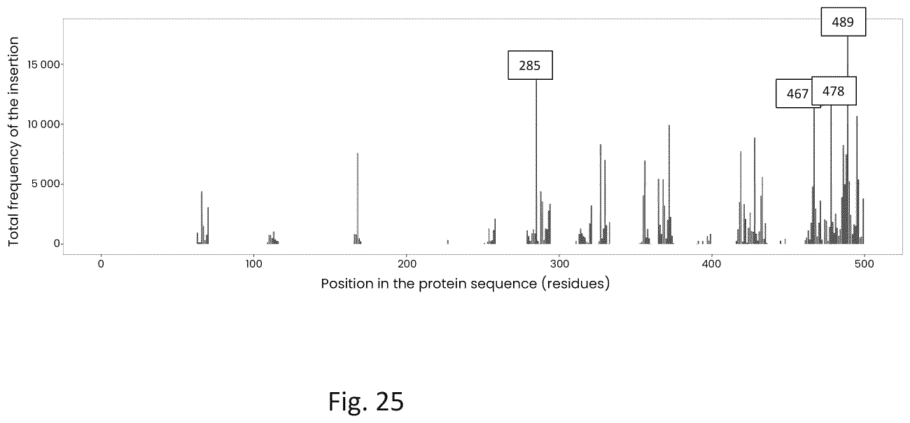

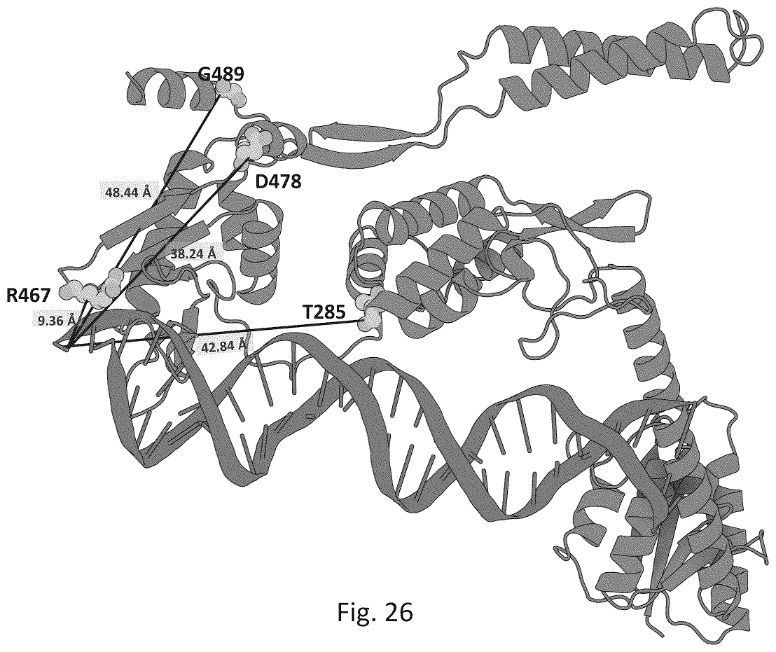

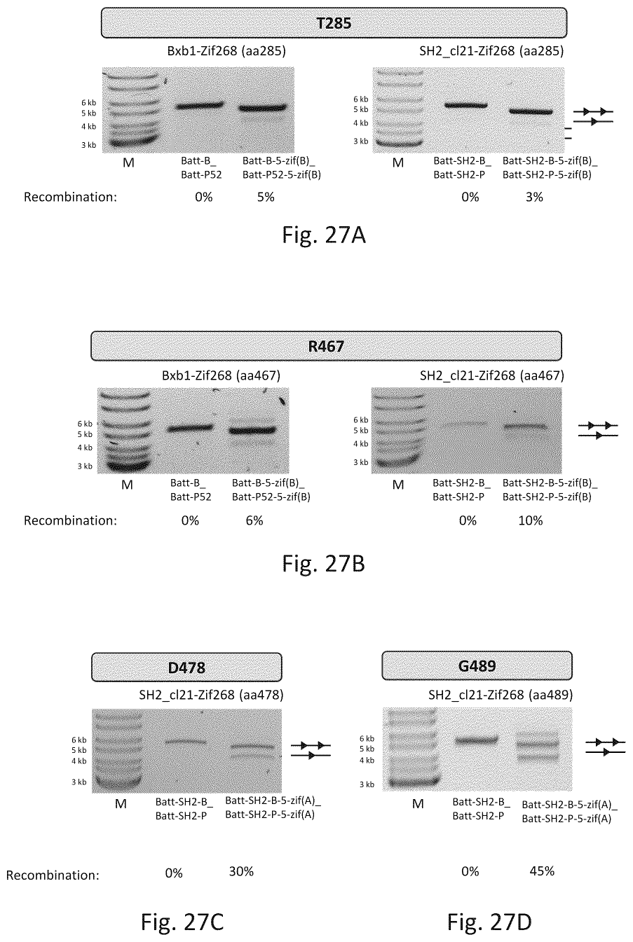

To develop the architecture for the insertional fusions with ZFDs, a library was created by fusing Zif268 between residues G278 and S279 in Brec (SEQ ID NO: 4) using two linkers, each consisting of one to eight GGS repeats (FIG. 8A). In addition, a library of lox-zif target sites was created where the spacing between the Brec1 recognition site (loxBTR, 34 bp) and the 9 bp Zif268 binding motif varied from 0 to 10 bp. Moreover, two different orientations of the Zif268 binding sequences relative to the loxBTR half-sites were included (FIG. 8B). To test all 1472 combinations, the designed fusion complexes were expressed on the target site library from the pEVO plasmid in E. coli and quantified the recombination efficiencies using nanopore sequencing by calculating the ratio of the recombined to non-recombined plasmids (FIG. 9).

Surprisingly, all variants carrying the insertional Zif268 fusion were inactive on the wt loxBTR target site, even at high induction level, indicating that the insertion of Zif268 between residues G278 and S279 disrupted the activity of Brec1. In sharp contrast, recombination activity on the loxBTR-n-zif target sites was observed, implying that binding of the ZFDs to their target site was required to recover recombination activity. The best performing variant (Zif268 fused to G278 and S279 of Brec1 via an 8×GGS linker each; construct referred to as Brec1²⁷⁸-Zif268; SEQ ID NO: 5) was further tested in a plasmid-based assay (schematically shown in FIG. 2B). Consistent with the screening results, the fusion complex showed impaired activity on the wt loxBTR, even at high induction level of 200 μg/ml L-arabinose, but regained full activity on the loxBTR-5-zif sites (FIG. 10A, 10B). The data shows that the insertional ZFD fusion to the recombinase generated a conditional recombinase that is dependent on binding of the ZFD to its target sequence for recombination activity.

To investigate functionality of the ZF-recombinase fusion in human cells, Brec or Brec1²⁷⁸-Zif268 expression constructs were transiently co-transfected together with fluorescent recombination-reporter plasmids into HEK293T cells (FIG. 1A). As expected, Brec1 did not distinguish between the loxBTR and loxBTR-5-zif target sites, and recombined both reporter plasmids at a rate of around 60%. The activity of Brec1²⁷⁸-Zif268 in HEK293T cells was severely impaired on the wt loxBTR plasmid, whereas almost full recombination activity was observed on the loxBTR-5-zif (B) (FIG. 11). Overall, these results indicate that the insertional ZF-recombinase fusion architecture generates recombination systems that require binding of the introduced DBDs for efficient recombination events also in human cells.

To test the versatility of the developed insertional fusion architecture, fusions of Zif268 with Vika, a recombinase isolated from Vibrio coralliilyticus (Karimova et al., 2013) (SEQ ID NO: 8) (FIG. 7), and three evolved Vika-based designer-recombinases (SEQ ID NOs: 14, 15 and 16) were created. Strikingly, observations similar to those obtained with the Cre-type Brec recombinase were made for the Vika constructs, when Zif268 was fused between residues 172 and 173 (SEQ ID NOs: 9 to 12), demonstrating the portability of the approach to other recombinase types (FIG. 12).

To test if the developed approach works with Large Serine Recombinases, fusions of Zif268 with Bxb1 recombinase and an evolved Bxb1-based designer-recombinase were created. Fusion of Zif268 between residues 285 and 286, residues 467 and 468, residues 478 and 479, and between residues 489 and 490 exhibited similar phenotype to the one observed with Cre-type and Vika-type recombinases (FIG. 27A to 27D).

To further test the applicability of the approach to other ZFDs and designer-recombinases, insertional fusions at the same position between residues 278 and 279 of Brec with ZFCCR5L (Perez et al., 2008) (SEQ ID NO: 13), which carries an additional zinc-finger and recognizes a 12 bp sequence, and Zif268 fusions with recombinases D7L (SEQ ID NO: 19), D7R (SEQ ID NO: 20) (Lansing et al., 2022), and RecHTLV (SEQ ID NO: 22) (Rojo-Romanos et al., 2023) were tested. All fusion complexes exhibited the conditional phenotype observed for Brec1²⁷⁸-Zif268 (FIGS. 13A to D). The results show that fusions of ZFDs within the recombinases create conditional-type recombinases depending not only on the recognition of the recombinase target sites, but also on the binding of the ZFD to its target site.

### Example 3: Relaxed-Type Recombinases can be Made More Specific by ZFD Fusions

The possibility of conditioning a relaxed specificity designer-recombinase by introducing an insertional ZFD was investigated. The recombinase RecHex (SEQ ID NO: 23) displays relaxed target site specificity and is capable of recombining a range of lox-like sites, including loxFlex1 (SEQ ID NO: 24), loxFlex2 (SEQ ID NO: 25), loxFlex3 (SEQ ID NO: 26), loxFlex4 (SEQ ID NO: 27), and loxFlex5 (SEQ ID NO: 28), which differ by 6 to 9 base pairs per half-site (FIG. 14A). These target sites exhibit only 31-54% sequence similarity to each other, suggesting that RecHex has the potential to recombine thousands of different target sequences. Based on these sequences, the loxFlex motif was determined. Through an extensive genome-wide investigation of loxHex motif occurrences within the human genome, a loxFlex-like target site situated within the MECP2 locus (loxMECP2, SEQ ID NO: 29) was identified on the human X chromosome. Duplication events at this specific genomic locus have been directly implicated in the onset of the MECP2 duplication syndrome, underscoring its therapeutic potential (Van Esch, 2012; D'Mello, 2021).

A RecFlex²⁷⁸-Zif268 fusion protein (SEQ ID NO: 30) was generated and its activity was assessed on the five lox-sites with and without flanking zif-motifs (FIG. 14B). Remarkably, the RecFlex²⁷⁸-Zif268 fusion disrupted activity on all five lox-sites, whereas recombination activity was restored in the presence of zif-motifs flanking these sites (FIG. 14C). Besides, RecFlex²⁷⁸-Zif268 also disrupted the activity on the loxMECP2 target, which was only restored in the presence of the zif-motifs flanking loxMECP2 (FIG. 14C). Notably, RecFlex²⁷⁸-Zif268 activity was increased at all lox-zif target sites compared with wild-type RecFlex, suggesting that the enzyme was not only conditioned for the flanking zif268 motif but also that the overall activity of the enzyme was enhanced, possibly by increased affinity for DNA provided by the ZFD. Results show that insertional ZFD fusions enable the programming of recombinases with only partial specificity to become highly specific within a short timeframe.

### Example 4: Design and Directed Evolution of ZFDs for a Genomic Locus

To test the present invention on a natural human genomic locus, it was applied to the heterodimeric designer-recombinase D7, recently developed for correcting the 140 kb genomic int1h-inversion causing hemophilia A (Lansing et al., 2022). Zinc fingers ZFL1 (SEQ ID NO: 31) and ZFL2 (SEQ ID NO: 32) for the human genomic sequence upstream of the loxF8 target sites in the F8 gene, and zinc fingers ZFR1 to ZFR4 (SEQ ID NOs: 33 to 36) for the downstream sequence were designed using publicly available platforms (Kim et al., 2009; Persikov et al., 2015) (FIG. 15A). The activity of the monomers (D7L (SEQ ID NO: 17) and D7R (SEQ ID NO: 18)) fused between positions 278 and 279 with the designed ZFDs (ZFL1, ZFL2, ZFR1, ZFR2, ZFR3, and ZFR4) was tested on the respective symmetric sites (loxF8L and loxF8R (SEQ ID NOs: 37 and 38)) and extended versions thereof, which included 20 bp of the genomic flanking sequences (loxF8L-flank and loxF8R-flank (SEQ ID NOs: 39 and 40)) (FIG. 15B). Consistent with previous results, none of the tested complexes showed activity on loxF8L and loxF8R target sites. In contrast, two of the fusion proteins (D7L-ZFL1 (SEQ ID NO: 91) and D7R-ZFR4 (SEQ ID NO: 92)) showed activity on the extended target sites, albeit at a lower efficiency when compared to the non-fused recombinases. To improve the designed ZFDs, a substrate-linked directed evolution (SLiDE) protocol was established for the directed evolution of the ZFDs as described in the following.

Zinc finger domains designed for the loxF8 flanking sequences were evolved based on the established substrate-linked directed evolution of recombinases (Buchholz & Stewart, 2001; Sarkar et al., 2007; Karpinski et al., 2016; Lansing et al., 2020 and 2022). SLiDE links excision activity of the lox-sites by a recombinase to the plasmid that encodes its gene. Because the activity of the recombinase was induced by ZFD binding to its target sites next to the lox-sites, this property was used for evolving the ZFDs in this system. A scheme of the procedure is depicted in FIG. 16. A library of the ZFDs was created by performing 50 cycles of error-prone PCR using MyTaq polymerase (Bioline), which lacks a proof-reading activity and therefore introduces mutations. The PCR products were digested with BbvCI and PspOMI and the band of around 400 bp for the zinc finger ZFL and around 500 bp for the zinc finger ZFR (this included in both cases the ZFs and the flanking linkers) was extracted from an agarose gel. This insert containing a ZF library was cloned into the digested pEVO vectors, that contained the loxF8L-flank (SEQ ID NO: 39) or loxF8R-flank (SEQ ID NO: 40) target sites and D7L (SEQ ID NO: 17) or D7R (SEQ ID NO: 18) recombinase sequences, respectively, with the insertion between the amino acids 278 and 279, that is flanked by BbvCI and PspOMI restriction sites. XL1-blue E. coli were transformed with the pEVO libraries and grown in 100 ml LB medium with chloramphenicol (30 μg/ml) and L-arabinose (200, 10, or 1 μg/ml). 10 ml of the culture was used for the plasmid extraction and 500 ng of plasmid DNA was digested with NdeI and AvrII, which restriction sites are located between the two lox target sites on the plasmid. Thereby, inactive variants which did not perform excision, were eliminated from the pool. The remaining active variants were amplified using error-prone PCR with primers binding upstream the Recombinase-ZF gene and downstream of the target site. The PCR product was digested with BbvCI and PspOMI to extract only the ZFD and its flanking linker sequences, and was cloned in the pEVO into the intact, wild type recombinase gene, as described above, thereby starting a new cycle of ZFD evolution. Additionally, to prevent the evolving ZFD from gaining a generally relaxed specificity, a counter-selection was performed on the loxF8L and loxF8R target sites, which did not have flanking ZF target sequences. For the counter-selection, the digested ZF library fragments were cloned into pEVO containing the D7L or D7R recombinase sequence and the loxF8L or loxF8R sites, respectively. In this case, a high L-arabinose concentration was used (200 μg/ml). After plasmid DNA extraction, error-prone PCR was performed, and inactive variants were amplified using the primers binding upstream of the ZF-recombinase gene and between the lox-sites. The cycling process was repeated with lowering ZF-recombinase expression levels on the flanked target sites (by lowering the concentration of L-arabinose) to select for most improved variants, while keeping it high on the lox-sites for counter-selection. Overall, 17 cycles of evolution on the flanked target sites and 3 cycles of counter-selection evolution on the lox-sites were performed for both ZFL and ZFR libraries. Finally, both recombinases fused with ZF libraries were combined, and the dimers inactive on the loxF8 target site (8 cycles) and active on the loxF8-flank sites (3 cycles) were selected in a similar way, as described in Hoersten et al. 2022, and in Lansing et al. 2022. A high-fidelity Herculase II Phusion DNA polymerase (Agilent) was used for dimer selection, in order to select the compatible combinations without introducing new mutations into the recombinase sequences. A substantial increase in recombination activity of the final libraries on the extended target sites was observed, showing that ZFDs with improved properties can be generated by using substrate-linked directed evolution (FIG. 17A).

For analyzing the sequence of the evolved ZFDs, active clones from the final ZFL (75 clones) and ZFR (59 clones) libraries, were picked and sent for E. coli overnight Sanger sequencing (Microsynth). The obtained sequences were analyzed to determine the mutational changes in the ZFD and linker sequence by comparing to the respective ZFL1 and ZFR4 sequences. The analysis was performed in R v4.1.0 using the dplyr, Sequence tools(https://github.com/ltschmitt/SequenceTools) and ggplot2 packages.

The sequence analyses of active clones in the final ZF libraries uncovered conserved acquired mutations (FIG. 17B). Some of these mutations were observed in the core helices of domain 1 in the ZFL library and domain 3 in the ZFR library. Conserved mutations in the scaffold of the ZFDs were also observed, as well as a conserved G-to-R mutation in the right (GGS)ₙ linker of both ZF libraries. To test whether this G-to-R mutation alone contributes to improved properties of the fusion proteins, the mutation was introduced into the right linker of the Brec1²⁷⁸-Zif268 complex (SEQ ID NO: 5). An increased recombination efficiency (2.5-fold) was observed for this construct on the loxBTR-5-zif (A) site (FIG. 18).

### Example 5: Recombinase-Zinc Finger Fusions Exhibit Improved Applied Properties

The two monomer libraries (D7L-ZFL and D7R-ZFR) were combined and several rounds of selection for activity of the recombinase heterodimer fused with the ZFDs were performed on the final extended loxF8 target site as it is found in the human genome. Clone G10 (also referred to as D7-ZF, a dimer comprising D7L-ZFL (SEQ ID NO: 41) and D7R-ZFR (SEQ ID NO: 42)) was selected for further studies because it showed high recombination activity on loxF8-flank and no activity on loxF8 (FIG. 20A). Sequence analysis revealed that during directed evolution, the zinc finger domains ZFL (SEQ ID NO: 43) and ZFR (SEQ ID NO: 44) acquired five and twelve mutations, respectively, as well as three mutations in the linkers, including two conserved G-to-R changes in the right linkers, which further contributed to their advantageous properties (FIG. 19).

To further investigate possible improvements of D7-ZF, it was tested on human genomic off-targets HG2 (SEQ ID NO: 45) and HG2L (SEQ ID NO: 46) that were reported to be recombined by the D7 heterodimer (Lansing et al., 2022). In contrast to D7, no activity was observed for the recombinase ZFD fusion D7-ZF on these off-targets or on their extended versions, which included the genomic sequences upstream and downstream of the 34 bp pseudo-loxF8-sites (FIG. 20B). To test whether D7-ZF possibly gained activity on new off-targets due to the presence of the additional DNA-binding domains, the human genome was bioinformatically screened for lox-sites with flanking sequences potentially recognized by the evolved ZFDs. D7 and D7-ZF were further tested on eight of the identified potential human off-targets (HGZF1 to HGZF8). D7 recombined two of these off-targets (HGZF4 and HGZF5), whereas no recombination activity was detected for D7-ZF on these sites, demonstrating its high specificity and showing that this approach does not lead to new off-targets (FIG. 20C). Altogether, these results demonstrate that insertional ZFD fusions within a designer-recombinase with therapeutic potential can improve its applied properties.

To investigate the construct's ability to perform inversion on the genomic locus in the F8 gene, D7 and D7-ZF expression plasmids (FIG. 1C) were transfected into HEK293T cells. HEK293T cells carry the F8 gene in the normal orientation, and successful recombination would invert it into the int1h disease orientation (FIG. 21A). As shown in FIGS. 21B and 21C, expression of the recombinases resulted in inversion of the loxF8 locus, with D7-ZF treatment improving the inversion efficiency noticeably. Finally, D7-ZF mRNA transfection of patient-derived F8 int1h-hiPSCs led to a 4-fold increase in inversion efficiency of the loxF8 locus over D7 (FIGS. 21D and 21E). The obtained results document the improved properties of D7-ZF, making it a preferred candidate for future therapeutic exploitation.

### Example 6: Activity of Brec1²⁷⁸-Zif268 in View of the Number of Zif Motifs

In the previously tested lox-zif target sites, both lox sites were flanked by the zif binding sites from the left and the right side, resulting in overall four zif motifs per two lox-sites. However, for some genomic targets it could be challenging to design ZFDs for all four different sequences flanking the target sites. It was therefore tested whether recombination activity can be observed in the presence of three, two or only one zif motif flanking the lox-target. To this end, target sites were developed where loxBTR is flanked by different combinations of zif binding sites (SEQ ID NOs: 62 and 63). Recombination activity of Brec1²⁷⁸-Zif268 (SEQ ID NO: 5) was tested on these sites. While the presence of only one out of four possible zif motifs flanking the loxBTR site led to some but low activity, two zif-motifs recovered approximately half of the recombination activity. Interestingly, almost full recovery of recombination activity was observed when three of the zif motifs were present, indicating that three out of four possible zif motifs flanking the recombinase target site are sufficient to obtain highest recombination rates (FIGS. 22A-22B). Although different combinations of the positions of the zif motifs were tested, according to the results from the recombination test, only the number of the zif motifs impact recombination efficiency and not the position of the zif motifs around the loxBTR target sites. Overall, these results indicate that efficiency of the ZF-Recombinase complex can be influenced by the number of zif motifs, which demonstrates the flexibility of potential genome targeting by the developed system.

### Example 7: Insertional Fusion of Brec1 with a TAL Domain

To test whether a different domain larger than a zinc finger domain can be fused within the recombinase sequence, Brec was fused at the selected position between residues G278 and S279 with a commercially available TAL2295 (Reyon et al., 2012) (SEQ ID NO: 47). TAL2295 binds to a 18 bp DNA sequence (SEQ ID NO: 48) and consists of 18 domains, resulting in a molecular size of 702 amino acids, which is almost eight times larger than the size of Zif268 (89 amino acids, SEQ ID NO: 49), and twice as large as a Cre-type recombinase (343 amino acids). For a proof-of-principle test, the same architecture developed for the Brec1-Zif268 fusion was used: (GGS)₈ linkers and 5 bp spacing between the target sites. The obtained fusion protein (SEQ ID NO: 51) was tested on both loxBTR-5-TAL2295 (A) and (B) target site orientations (SEQ ID NOs: 52, 53), as well as on the loxBTR target site (SEQ ID NO: 65) alone (FIG. 23A). The results revealed that activity of Brec was drastically disrupted by insertion of the TAL2295, however, despite the significantly larger size of the insertion, some activity could be restored when the TAL2295 binding sites (SEQ ID NO: 48) were flanking the loxBTR site in the orientation B (FIG. 23B). Although the resulting recombination efficiency on that target site was not as high as that for the zinc finger fusions, the present results indicate that insertional fusion of TALE domains within the recombinase sequence can be used to create conditional recombinases.

### CITED NON-PATENT LITERATURE

Adikusuma, F. et al. (2018). Large deletions induced by Cas9 cleavage. Nature 560, E8-E9.
Anzalone, A. V., Koblan, L. W., Liu, D. R. (2020). Genome editing with CRISPR-Cas nucleases, base editors, transposases and prime editors. Nat. Biotechnol. 38, 824-844.
Bhakta, M. S., Segal, D. J. (2010). The generation of zinc finger proteins by modular assembly. Methods Mol Biol. 649, 3-30.
Boch, J. et al. (2009). Breaking the code of DNA binding specificity of TAL-type III effectors. Science. 326(5959), 1509-1512.
Bogdanove, A. J. & Voytas, D. F. TAL effectors: customizable proteins for DNA targeting. Science. 333(6051), 1843-1846.
Buchholz, F. & Hauber, J. (2011). In vitro evolution and analysis of HIV-1 LTR-specific recombinases. Methods 53, 102-109.
Buchholz, F., and Stewart, A. F. (2001). Alteration of Cre recombinase site specificity by substrate-linked protein evolution. Nat Biotechnol 19, 1047-1052.
Carroll, D. (2017). Genome Editing: Past, Present, and Future. Yale J. Biol. Med. 90, 653-659.
Cassandri, M. et al. (2017). Zinc-finger proteins in health and disease. Cell Death Discov. 3, 17071
Christy, B. & Nathans, D. DNA binding site of the growth factor-inducible protein Zif268. Proc. Natl. Acad. Sci. 86, 8737-8741 (1989).
D'Mello, S. R. (2021). MECP2 and the biology of MECP2 duplication syndrome. J. Neurochem. 159, 29-60.
Elrod-Erickson, M., Rould, M. A., Nekludova, L. and Pabo, C. O. (1996). Zif268 protein-DNA complex refined at 1.6å: a model system for understanding zinc finger-DNA interactions. Structure 4, 1171-1180.
Enache, O. M. et al. (2020). Cas9 activates the p53 pathway and selects for p53-inactivating mutations. Nat. Genet. 52, 662-668.
Engler, C., Kandzia, R., Marillonnet, S. (2008). A one pot, one step, precision cloning method with high throughput capability. PLoS One 3(11):e3647.
Ennifar, E. (2003). Crystal structure of a wild-type Cre recombinase-loxP synapse reveals a novel spacer conformation suggesting an alternative mechanism for DNA cleavage activation. Nucleic Acids Res. 31, 5449-5460.
Grant, C. E., Bailey, T. L. and Noble, W. S. (2011). FIMO: scanning for occurrences of a given motif. Bioinformatics 27, 1017-1018.
Haapa, S. et al. (1999). An efficient and accurate integration of mini-Mu transposons in vitro: a general methodology for functional genetic analysis and molecular biology applications. Nucleic Acids Res. 27 (13), 2777-2784.
Hayes, F. & Hallet, B. (2000). Pentapeptide scanning mutagenesis: encouraging old proteins to execute unusual tricks. Trends Microbiol. 8, 571-577.
Ichikawa, D. M. et al. (2023). A universal deep-learning model for zinc finger design enables transcription factor reprogramming. Nat Biotechnol. 41, 1117-1129.
Jelicic, M. et al. (2023). Discovery and characterization of novel Cre-type tyrosine site-specific recombinases for advanced genome engineering. Nucleic Acids Res. 51(10), 5285-5297.
Jinek, M. et al. (2016). A Programmable Dual-RNA-Guided DNA Endonuclease in Adaptive Bacterial Immunity. Science. 337, 816-821. Karimova, M. et al. (2013). Vika/vox, a novel efficient and specific Cre/loxP-like site-specific recombination system. Nucleic Acids Res. 41, e37-e37.
Karpinski, J., Hauber, I., Chemnitz, J., Schafer, C., Paszkowski-Rogacz, M., Chakraborty, D., Beschorner, N., Hofmann-Sieber, H., Lange, U. C., Grundhoff, A., et al. (2016). Directed evolution of a recombinase that excises the provirus of most HIV-1 primary isolates with high specificity. Nat Biotechnol, 34, 401-409.
Kim, H. J., Lee, H. J., Kim, H., Cho, S. W. & Kim, J.-S (2009). Targeted genome editing in human cells with zinc finger nucleases constructed via modular assembly. Genome Res. 19, 1279-1288.
Kosicki, M., Tomberg, K. & Bradley, A. (2018). Repair of double-strand breaks induced by CRISPR-Cas9 leads to large deletions and complex rearrangements. Nat. Biotechnol. 36, 765-771.
Landschulz, W. H. et al. (1988). The leucine zipper: a hypothetical structure common to a new class of DNA binding proteins. Science. 240(4860), 1759-1764.
Lansing, F. et al. (2020). A heterodimer of evolved designer-recombinases precisely excises a human genomic DNA locus. Nucleic Acids Res. 48, 472-485.
Lansing, F. et al. (2022). Correction of a Factor VIII genomic inversion with designer-recombinases. Nat. Commun. 13, 422.
Leibowitz, M. L. et al. (2021). Chromothripsis as an on-target consequence of CRISPR-Cas9 genome editing. Nat. Genet. 53, 895-905.
Maeder, M. L. et al. (2008). Rapid “open-source” engineering of customized zinc-finger nucleases for highly efficient gene modification. Mol Cell. 31, 294-301.
Matthews, K. S. & Nichols, J. C. Lactose Repressor Protein: Functional Properties and Structure. Progress in Nucleic Acid Research and Molecular Biology. Academic Press. 58, 127-164. ISSN 0079-6603, ISBN 9780125400589.
Meinke, G., Bohm, A., Hauber, J., Pisabarro, M. T. and Buchholz, F. (2016). Cre Recombinase and Other Tyrosine Recombinases. Chem Rev, 116, 12785-12820.
Mirdita, M. et al. (2022). ColabFold: making protein folding accessible to all. Nat. Methods 19, 679-682.
Nunez, N. et al. (2011). The multi-zinc finger protein ZNF217 contacts DNA through a two-finger domain. J Biol Chem. 286(44), 38190-38201.
Papathanasiou, S. et al. (2021). Whole chromosome loss and genomic instability in mouse embryos after CRISPR-Cas9 genome editing. Nat. Commun. 12, 5855.
Perez, E. E. et al. (2008). Establishment of HIV-1 resistance in CD4⁺ T cells by genome editing using zinc-finger nucleases. Nat. Biotechnol. 26, 808-816.
Persikov, A. V. et al. (2015). A systematic survey of the Cys₂His2 zinc finger DNA-binding landscape. Nucleic Acids Res. 43, 1965-1984.
Persikov, A. V. & Singh, M. (2014). De novo prediction of DNA-binding specificities for Cys₂His2 zinc finger proteins. Nucleic Acids Res. 42, 97-108.
Petyuk, V., McDermott, J., Cook, M. & Sauer, B. (2004). Functional Mapping of Cre Recombinase by Pentapeptide Insertional Mutagenesis. J. Biol. Chem. 279, 37040-37048.
Richter, A. et al. (2014). A TAL effector repeat architecture for frameshift binding. Nat Commun. 5, 3447.
Rutherford, K., Yuan, P., Perry, K., Sharp, R., Van Duyne, G. D. (2013). Attachment site recognition and regulation of directionality by the serine integrases. Nucleic Acids Research. 41 (17), 8341-8356.
Rojo-Romanos, T. et al. (2023). Precise excision of HTLV-1 provirus with a designer-recombinase. Mol. Ther. S1525001623001351 doi:10.1016/j.ymthe.2023.03.014.
Sander, J. D. et al. (2011). Selection-free zinc-finger-nuclease engineering by context-dependent assembly (CoDA). Nat Methods. 8, 67-69.
Sarkar, I., Hauber, I., Hauber, J. & Buchholz, F. (2007). HIV-1 Proviral DNA Excision Using an Evolved Recombinase. Science 316, 1912-1915.
Schmitt, L. T., Paszkowski-Rogacz, M., Jug, F. & Buchholz, F. (2022). Prediction of designer-recombinases for DNA editing with generative deep learning. Nat. Commun. 13, 7966.
Schnarr, M. et al. (1991). DNA binding properties of the LexA repressor. Biochimie. 73(4), 423-431.
Sinha, S. et al. (2021). A systematic genome-wide mapping of oncogenic mutation selection during CRISPR-Cas9 genome editing. Nat. Commun. 12, 6512.
Tomasello, G., Armenia, I. & Molla, G. (2020). The Protein Imager: a full-featured online molecular viewer interface with server-side HQ-rendering capabilities. Bioinformatics 36, 2909-2911.
Van Duyne, G. D., Rutherford, K. (2013). Large serine recombinase domain structure and attachment site binding. Crit Rev Biochem Mol Biol 48(5), 476-91.
Van Esch, H. (2012). MECP2 Duplication Syndrome. Mol. Syndromol. 2, 128-136.
Wang, J. Y. & Doudna, J. A. (2023). CRISPR technology: A decade of genome editing is only the beginning. Science 379, eadd8643.
Xu, X. & Qi, L. S. (2019). A CRISPR-dCas Toolbox for Genetic Engineering and Synthetic Biology. J Mol Biol. 431, 34-47.
Zetsche, B. et al. (2015). Cpf1 Is a Single RNA-Guided Endonuclease of a Class 2 CRISPR-Cas System. Cell. 163, 759-771.

1. A method of identifying an amino acid position of a DNA modifying enzyme for insertion of a heterologous DNA binding domain (DBD), the method comprising the steps of:
(i) providing a library of DNA modifying enzymes, wherein the members of the library comprise heterologous amino acid sequence insertions throughout the DNA modifying enzyme;
(ii) identifying those DNA modifying enzymes of the library that have DNA modifying activity; and
(iii) identifying the position of the insertion in those DNA modifying enzymes identified in step (ii).

2. The method according to claim 1, wherein the one or more positions of the insertions identified in step (iii) are mapped to structural data of the DNA modifying enzyme and/or wherein the library of DNA modifying enzymes provided in (i) is encoded by a nucleic acid library, optionally wherein step (iii) comprises determining at least part of the nucleic acid sequence encoding those DNA modifying enzymes identified in step (ii), and/or
the method further comprising the step of selecting one or more amino acid positions for insertion of the heterologous DBD that are surface exposed in the DNA modifying enzyme and in proximity to the DNA binding site of the DNA modifying enzyme, and/or
wherein the heterologous amino acid sequence comprised in each of the members of the library of DNA modifying enzymes independent of each other have a length of between three and ten amino acids, optionally a length of five amino acids.

3. A method of producing a DNA modifying enzyme comprising an insertion of a heterologous DBD, the method comprising the steps of:
(i) inserting a nucleic acid sequence encoding the heterologous DBD into a nucleic acid sequence encoding the DNA modifying enzyme at the nucleotide triplet(s) encoding the one or more positions identified in the method of claims 1 or 2; and
(ii) expressing the nucleic acid sequence produced in step (i).

4. The method according to claim 3, wherein the nucleic acid sequence encoding the heterologous DBD further comprises a nucleic acid sequence encoding a peptide linker upstream and a peptide linker downstream of the nucleic acid sequence encoding the heterologous DBD; optionally wherein the linker is a glycine-serine linker, optionally a glycine-serine linker with at least one G to R substitution.

5. The method according to any one of claims 1 to 4, wherein the DBD is a zinc-finger (ZF) DBD or a transcription activator-like effector (TALE) DBD, and/or wherein the DNA modifying enzyme is a transposase or a recombinase, optionally a serine recombinase or a tyrosine recombinase, optionally a tyrosine recombinase.

6. A DNA modifying enzyme comprising an insertion of a heterologous DBD,
obtained by a method according to any one of claims 3 to 5, and/or
wherein
(i) the DNA modifying enzyme is Cre or a Cre-derived recombinase and the DBD is inserted between amino acid positions 278 and 279; or
(ii) the DNA modifying enzyme is Vika or a Vika-derived recombinase and the DBD is inserted between amino acid positions 172 and 173; or
(iii) the DNA modifying enzyme comprises an amino acid sequence having at least 80% sequence identity to any one of SEQ ID NOs: 5, 9 to 13, 19, 20, 22, 30, 41, 42, 51, 64 and 91 to 200.

7. A DNA modifying enzyme comprising an insertion of a heterologous DBD, wherein the DBD optionally comprises at its N- and/or C-terminus a peptide linker, wherein the DNA modifying enzyme is inactive on its target site when the heterologous DBD does not bind to its target DNA, and wherein the DNA modifying enzyme is active on its target site when the heterologous DBD binds to its target DNA.

8. A nucleic acid or a plurality of nucleic acids encoding the DNA modifying enzyme according to claim 6 or 7.

9. An expression vector comprising the nucleic acid or plurality of nucleic acids according to claim 8.

10. A host cell or culture of host cells comprising the nucleic acid or plurality of nucleic acids according to claim 8, or the expression vector according to claim 9, optionally wherein the host cell expresses the DNA modifying enzyme encoded by the nucleic acid or plurality of nucleic acids.

11. A pharmaceutical composition comprising the DNA modifying enzyme according to any one of claims 6 or 7, the nucleic acid or plurality of nucleic acids according to claim 8, the expression vector according to claim 9, or the host cell or culture of host cells according to claim 10, and a pharmaceutically acceptable excipient or carrier.

12. (canceled)

13. A method for modifying a nucleic acid sequence of interest, comprising contacting a cell or tissue comprising the nucleic acid sequence of interest with the DNA modifying enzyme according to any one of claims 6 or 7, the nucleic acid or plurality of nucleic acids according to claim 8, the expression vector according to claim 9, the host cell or culture of host cells according to claim 10, or the pharmaceutical composition according to claim 11 under conditions allowing the DNA modifying enzyme to modify the nucleic acid sequence of interest.

14. Method of changing the specificity and/or activity of a DNA modifying enzyme comprising the steps of:
(i) identifying an amino acid position of a DNA modifying enzyme for insertion of a heterologous DBD according to the method of claim 1 or 2; and
(ii) inserting a heterologous DBD at the position identified in step (i).

15. Method of evolving a DBD on a target sequence of interest, comprising the steps of:
(i) creating a library of variants of the DBD;
(ii) cloning the library of step (i) into expression vectors comprising a first region encoding a DNA recombining enzyme, a second region comprising a first target site of said DNA recombining enzyme and regions flanking said first target site, and a third region comprising a second target site of said DNA recombining enzyme and regions flanking said second target site, such that a DBD is inserted directly or via peptide linkers into the DNA recombining enzyme, wherein the first, second and third regions are separated from another;
(iii) introducing the expression vectors into host cells and culturing the host cells, thereby expressing the encoded DNA recombining enzyme comprising the DBD;
(iv) isolating plasmids from the cell culture of step (iii) and determining whether the DNA recombining enzyme catalyzed a recombination reaction at both target sites on the vector;
(v) amplifying the DBD of those plasmids that were found to encode a DNA recombining enzyme comprising the DBD showing recombination activity using error-prone PCR to generate a new library of variants of the DBD;
(vi) repeating steps (ii) to (iv) with the library of step (v).

16. A DNA modifying enzyme comprising an insertion of a heterologous DNA Binding Domain (DBD), wherein:
(i) the DNA modifying enzyme is Cre or a Cre-derived recombinase and the DBD is inserted between amino acid positions 278 and 279; or
(ii) the DNA modifying enzyme is Vika or a Vika-derived recombinase and the DBD is inserted between amino acid positions 172 and 173; or
(iii) the DNA modifying enzyme comprises an amino acid sequence having at least 80% sequence identity to any one of SEQ ID NOs: 5, 9 to 13, 19, 20, 22, 30, 41, 42, 51, 64 and 91 to 200.


SEQUENCE LISTING (226 sequences)

SEQ ID NO: 1-2 - skipped (2 entries)

SEQ ID NO: 3 - DNA, 27 bp, unidentified
  source 1..27: mol_type = other DNA
tataaaccccctccaaccaggtgctaa

SEQ ID NO: 4 - protein, 343 aa, unidentified
MSILLTLHQSLSALLVDATSDEARKNLMDVLRDRQAFSERTWKVLLSVCRTWAAWCKLNNRKWFPAEPEDVRDYLLHLQARGLAVNTILQHLAQLNMLHRRFGLPRPGDSDAVSLVMRRIRRENVDAGERTKQALAFERTDFDQVRALMENSERGQDIRTLALLGVAYNTLLRVSEIARIRIKDISRTDGGRMLIHISRTKTLVSTAGVEKALSLGVTKLVERWISVSGVASDPNNYLFCQVRINGVAVPSATSRLSTDVLRKIFEAAHRLIYGAKDGSGQRYLAWSGHSARVGAARDMARAGVSIAEIMQAGGWTTVESVMNYIRNLDSETGAMVRLLEDGD

SEQ ID NO: 5 - protein, 490 aa, synthetic construct
MSILLTLHQSLSALLVDATSDEARKNLMDVLRDRQAFSERTWKVLLSVCRTWAAWCKLNNRKWFPAEPEDVRDYLLHLQARGLAVNTILQHLAQLNMLHRRFGLPRPGDSDAVSLVMRRIRRENVDAGERTKQALAFERTDFDQVRALMENSERGQDIRTLALLGVAYNTLLRVSEIARIRIKDISRTDGGRMLIHISRTKTLVSTAGVEKALSLGVTKLVERWISVSGVASDPNNYLFCQVRINGVAVPSATSRLSTDVLRKIFEAAHRLIYGAKDGTSAGGSGGSGGSGGSGGSGGSGGSGGSDRERPYACPVESCDRRFSRSDELTRHIRIHTGQKPFQCRICMRNFSRSDHLTTHIRTHTGEKPFACDICGRKFARSDERKRHTKIHLRQKDGRAGGSGGSGGSGGSGGSGGSGGSGGSGPSGQRYLAWSGHSARVGAARDMARAGVSIAEIMQAGGWTTVESVMNYIRNLDSETGAMVRLLEDGD

SEQ ID NO: 6 - protein, 24 aa, synthetic construct
GGSGGSGGSGGSGGSGGSGRSGGS

SEQ ID NO: 7 - protein, 24 aa, synthetic construct
GGSGGSGGSGRSGGSGRSGGSGGS

SEQ ID NO: 8 - protein, 361 aa, unidentified
MTDLTPFPPLEHLEPDEFADLVRKAIKRDPQAGAHPAIQSAISHFQDEFVRRQGEWQPATLQRLRNAWNVFVRWCTHQGIPALPARHQDVERYLIERRNELHRNTLKVHLWAIGKTHVISGLPNPCAHRYVKAQMAQITHQKVRERERIEQAPAFRESDLDRLTELWSATRSVTQQRDLMIVSLAYETLLRKNNLEQMKVGDIEFCQDGSALITIPFSKTNHSGRDDVRWISPQVANQVHAYLQLPNIDADPQCFLLQRVKRSGKALNPESHNTLNGHHPVSEKLISRVFERAWRALNHETGPRYTGHSARVGAAQDLLQEGYSTLQVMQAGGWSSEKMVLRYGRHLHAHTSAMAQKRRQR

SEQ ID NO: 9 - protein, 503 aa, synthetic construct
MTDLTPFPPLEHLEPDEFADLVRKAIKRDPQAGAHPAIQSAISHFQDEFVRRQGEWQPATLQRLRNAWNVFVRWCTHQGIPALPARHQDVERYLIERRNELHRNTLKVHLWAIGKTHVISGLPNPCAHRYVKAQMAQITHQKVRERERIEQAPAFRESDLDRLTELWSATRSLEGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSLYNVTQQRDLMIVSLAYETLLRKNNLEQMKVGDIEFCQDGSALITIPFSKTNHSGRDDVRWISPQVANQVHAYLQLPNIDADPQCFLLQRVKRSGKALNPESHNTLNGHHPVSEKLISRVFERAWRALNHETGPRYTGHSARVGAAQDLLQEGYSTLQVMQAGGWSSEKMVLRYGRHLHAHTSAMAQKRRQR

SEQ ID NO: 10 - protein, 503 aa, synthetic construct
MTDLTPFPPLEHLEPDEFADLVRKAIKRDPQAGAHPAIQSAISHFQDEFVRRQGAWQPATLRRLRNAWNVFVRWCTLQGIPALPARHQDVERYLIERRNELHRNTLKVHLWAIGKTHVISGLPNPCAHRYVKAQMAQITHQKVRERERIKQAPAFRESDLDRLTELWSATRSLEGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSLYNVTQQRDLMIVSLAYETLLRKNNLEQMRVGDIEFCQDGSALITIPFSKTNHSGRDDVRWISPQVANQVRAYLQLPNIDADPQCFLLQRAGRSGRALNPESHNTLGGHHPVSEKLISRVFERAWRALNHGTGPRYTGHSARVGATLDLLQEGYSTLQVMQAGGWSSEKMVLRYGRHLHAHSSAMAQKRRQR

SEQ ID NO: 11 - protein, 503 aa, synthetic construct
MTDMTPFPPLEHLEPDEFADLVREAIKRDPQAGAHPAIQSAISHFQDEFVRRQGELQPATLQRLRYAWNVFVRWCTHRGIQALPARHQDVERYLIERRNELHRKTLKVHLWAIGRTHVISGLPNPCAHRYVKAQMAQITHQKVRERERIKQAPAFRESDLVRLTELWSATSSLEGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSLYNVTQQRDLMIISLAYETLLRKSNLEQMKVGDIEFCQDGSALITIPFSKTNHSGRDDVRWISPQVANLVRAYLQLPGVNADPQCFLLQRVRRSGKALNQEGHNTLNGHHPVGEKLIGLVFERAWRALGHETGPRYTGHSARVGAAQDLLQEGYSTLQVMQAGGWSSEEMVLRYGRHLLAHNSAMAQKRRQR

SEQ ID NO: 12 - protein, 503 aa, synthetic construct
MTELTPFPPLEHLEPDEFADLVRKAIKRDPHAGAHPAIQRAISHFQDEFVRRQGELQPTTLRRLRYAWNDFVRWCTRQGILALPARHQDVERYLIERSSGLHRNTLKANLWAIGKIHVISGLPNPCAHRHVKAQMAQIAHQKVRERERIRQAPAFRESDLERLTELWSGTRSLEGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSLYNVIQQRDLMIISLAYETLLRKNNLEQMKVGDIEFCQDGSALITIPFSKTNHSGRDDVRWISPRVASQVRAYLQLPNVDADPQCFLLQRVVRSGKALSPEGHNTLDGHHPVSGMLISSVFERAWRALGHEAGPRYTGHSARVGAAQDLLQEGYSILQVMQAGGWSSEEMVLRYGRHLHARNSAMAQKRRQR

SEQ ID NO: 13 - protein, 511 aa, synthetic construct
MSILLTLHQSLSALLVDATSDEARKNLMDVLRDRQAFSERTWKVLLSVCRTWAAWCKLNNRKWFPAEPEDVRDYLLHLQARGLAVNTILQHLAQLNMLHRRFGLPRPGDSDAVSLVMRRIRRENVDAGERTKQALAFERTDFDQVRALMENSERGQDIRTLALLGVAYNTLLRVSEIARIRIKDISRTDGGRMLIHISRTKTLVSTAGVEKALSLGVTKLVERWISVSGVASDPNNYLFCQVRINGVAVPSATSRLSTDVLRKIFEAAHRLIYGAKDGLEGGSGGSGGSGGSGGSGGSGGSGGSERPFQCRICMRNFSDRSNLSRHIRTHTGEKPFACDICGRKFAISSNLNSHTKIHTGSQKPFQCRICMRNFSRSDNLARHIRTHTGEKPFACDICGRKFATSGNLTRHTKIHLRGSGGSGGSGGSGGSGGSGGSGGSGGSLYSSGQRYLAWSGHSARVGAARDMARAGVSIAEIMQAGGWTTVESVMNYIRNLDSETGAMVRLLEDGD

SEQ ID NO: 14 - protein, 361 aa, unidentified
MTDLTPFPPLEHLEPDEFADLVRKAIKRDPQAGAHPAIQSAISHFQDEFVRRQGAWQPATLRRLRNAWNVFVRWCTLQGIPALPARHQDVERYLIERRNELHRNTLKVHLWAIGKTHVISGLPNPCAHRYVKAQMAQITHQKVRERERIKQAPAFRESDLDRLTELWSATRSVTQQRDLMIVSLAYETLLRKNNLEQMRVGDIEFCQDGSALITIPFSKTNHSGRDDVRWISPQVANQVRAYLQLPNIDADPQCFLLQRAGRSGRALNPESHNTLGGHHPVSEKLISRVFERAWRALNHGTGPRYTGHSARVGATLDLLQEGYSTLQVMQAGGWSSEKMVLRYGRHLHAHSSAMAQKRRQR

SEQ ID NO: 15 - protein, 361 aa, unidentified
MTDMTPFPPLEHLEPDEFADLVREAIKRDPQAGAHPAIQSAISHFQDEFVRRQGELQPATLQRLRYAWNVFVRWCTHRGIQALPARHQDVERYLIERRNELHRKTLKVHLWAIGRTHVISGLPNPCAHRYVKAQMAQITHQKVRERERIKQAPAFRESDLVRLTELWSATSSVTQQRDLMIISLAYETLLRKSNLEQMKVGDIEFCQDGSALITIPFSKTNHSGRDDVRWISPQVANLVRAYLQLPGVNADPQCFLLQRVRRSGKALNQEGHNTLNGHHPVGEKLIGLVFERAWRALGHETGPRYTGHSARVGAAQDLLQEGYSTLQVMQAGGWSSEEMVLRYGRHLLAHNSAMAQKRRQR

SEQ ID NO: 16 - protein, 361 aa, unidentified
MTELTPFPPLEHLEPDEFADLVRKAIKRDPHAGAHPAIQRAISHFQDEFVRRQGELQPTTLRRLRYAWNDFVRWCTRQGILALPARHQDVERYLIERSSGLHRNTLKANLWAIGKIHVISGLPNPCAHRHVKAQMAQIAHQKVRERERIRQAPAFRESDLERLTELWSGTRSVIQQRDLMIISLAYETLLRKNNLEQMKVGDIEFCQDGSALITIPFSKTNHSGRDDVRWISPRVASQVRAYLQLPNVDADPQCFLLQRVVRSGKALSPEGHNTLDGHHPVSGMLISSVFERAWRALGHEAGPRYTGHSARVGAAQDLLQEGYSILQVMQAGGWSSEEMVLRYGRHLHARNSAMAQKRRQR

SEQ ID NO: 17 - protein, 343 aa, synthetic construct
MSNLQTLHQNLSALLANATSDEARKNLMDVFRDRRAFSEATWKTLLSVCRTWAAWCKLNNRKWFPAEPEDVRDYLLHLQVRGLAVNTIQRHLALLNMLHRRSGLPRPGDSSAVSLVMRRIRKENVDAGERVRQALAFERTDFDKVRSLMGNSDRCQDIRNLAFLGVAYNTLLRISEIARIRIKDISRTDGGRMLIHIGRTKTLVSTAGVEKALSLGVTRLVGRWISVSGVAGDPNNYLFCRVRKNGVAAPSATSQLSTDVLRGVFAAAHRLVYGTKDDSGQGYLTWSGHSARVGAARDMARAGVSIAEIMQAGGWTTVESVMSYLRNLDSETGAMVRLLEDGD

SEQ ID NO: 18 - protein, 343 aa, synthetic construct
MSNIQTPHQSLSALLTDATSDVTRKNLADMFRDSQAFSEHTWKMLLSVCRSWAAWCELNNRKWLPVEPEDVRDYLLHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLVYGAKGDSGQKYLAWSGHSARVGAARDMARAGVPIPEIMQAGGWTTVNSVMNYIRNLDSETGAMVRLLEDSD

SEQ ID NO: 19 - protein, 485 aa, synthetic construct
MSNLQTLHQNLSALLANATSDEARKNLMDVFRDRRAFSEATWKTLLSVCRTWAAWCKLNNRKWFPAEPEDVRDYLLHLQVRGLAVNTIQRHLALLNMLHRRSGLPRPGDSSAVSLVMRRIRKENVDAGERVRQALAFERTDFDKVRSLMGNSDRCQDIRNLAFLGVAYNTLLRISEIARIRIKDISRTDGGRMLIHIGRTKTLVSTAGVEKALSLGVTRLVGRWISVSGVAGDPNNYLFCRVRKNGVAAPSATSQLSTDVLRGVFAAAHRLVYGTKDDLEGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSLYSSGQGYLTWSGHSARVGAARDMARAGVSIAEIMQAGGWTTVESVMSYLRNLDSETGAMVRLLEDGD

SEQ ID NO: 20 - protein, 485 aa, synthetic construct
MSNIQTPHQSLSALLTDATSDVTRKNLADMFRDSQAFSEHTWKMLLSVCRSWAAWCELNNRKWLPVEPEDVRDYLLHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLVYGAKGDLEGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSLYSSGQKYLAWSGHSARVGAARDMARAGVPIPEIMQAGGWTTVNSVMNYIRNLDSETGAMVRLLEDSD

SEQ ID NO: 21 - protein, 343 aa, unidentified
MSKLLTLHQDLSALLADVTSDEARKNLMDMFRDRQAFPEHTWEMLLSVCRSWAAWCESNNRKWFPAEPEDVRDYLLHLQARGLTVNTVQKHLAELNTLHRRSGLPRPGDSNAVTLVMRRIRRENVDAGERAKQALAFERTDFDRVRSLMENSDRCLDIRNLAFLGVAYNTLLRISEIARIRVKDISRTDGGRMLIHIGRTKTLVSAAGVEKALSLGVTKLVERWITASGVADDPNNYLFCRVRRYGVVVSSATSRLSTHAMQGIFGTAHRLIYGAKDDSGQRYLAWSGHSARVGAARDLARAGVSIAEIMQAGGWTRVNSVMNYIRNLDSETGAMVRLLEDGD

SEQ ID NO: 22 - protein, 490 aa, synthetic construct
MSKLLTLHQDLSALLADVTSDEARKNLMDMFRDRQAFPEHTWEMLLSVCRSWAAWCESNNRKWFPAEPEDVRDYLLHLQARGLTVNTVQKHLAELNTLHRRSGLPRPGDSNAVTLVMRRIRRENVDAGERAKQALAFERTDFDRVRSLMENSDRCLDIRNLAFLGVAYNTLLRISEIARIRVKDISRTDGGRMLIHIGRTKTLVSAAGVEKALSLGVTKLVERWITASGVADDPNNYLFCRVRRYGVVVSSATSRLSTHAMQGIFGTAHRLIYGAKDDTSAGGSGGSGGSGGSGGSGGSGGSGGSDRERPYACPVESCDRRFSRSDELTRHIRIHTGQKPFQCRICMRNFSRSDHLTTHIRTHTGEKPFACDICGRKFARSDERKRHTKIHLRQKDGRAGGSGGSGGSGGSGGSGGSGGSGGSVNSGQRYLAWSGHSARVGAARDLARAGVSIAEIMQAGGWTRVNSVMNYIRNLDSETGAMVRLLEDGD

SEQ ID NO: 23 - protein, 343 aa, synthetic construct
MSKLQTIHQDLSALLVDVTSDEARRNLMDVLRDHQALSKHTWRVLLSVCRSWAAWCELNNRKWFPAEPEDVRDYLLHLQTRGLTVNTIQQHLCQLNLLHRRSGLPRPGDSNAVSLVMRRIRKENIDAGERVKQALAFERTDFDQVRSLMENSDRCQDIRNLAFLGVAYNTLLRISEIARIRVRDITRTDGGRMLIHIGRTKTLVSAAGEEKALSLGVTKLVERWISVSGVADDRNNYLFCRVKRNGVAAPSAFSQLSTPALHGVFAAAHRLIHGAKDASGQRYLTWSGHSARVGAARDMARAGVPVAEIMQAGGWTTVESVMRYLRNLDSETGAMVRLLEDGD

SEQ ID NO: 24 - DNA, 34 bp, synthetic construct
  source 1..34: mol_type = other DNA
ctcattacatttaaccaaaattaaatgtaatgag

SEQ ID NO: 25 - DNA, 34 bp, synthetic construct
  source 1..34: mol_type = other DNA
ttatattgtgataaccaaaattatcacaatataa

SEQ ID NO: 26 - DNA, 34 bp, synthetic construct
  source 1..34: mol_type = other DNA
ccatcttttgttagatttgaataacaaaagatgg

SEQ ID NO: 27 - DNA, 34 bp, synthetic construct
  source 1..34: mol_type = other DNA
tacacagtgtatattgatttttatacattgtgta

SEQ ID NO: 28 - DNA, 34 bp, synthetic construct
  source 1..34: mol_type = other DNA
ataacctaatataattgtatttatattaggtcag

SEQ ID NO: 29 - DNA, 34 bp, synthetic construct
  source 1..34: mol_type = other DNA
cacactttgttttatgtaggctataccttgataa

SEQ ID NO: 30 - protein, 490 aa, synthetic construct
MSKLQTIHQDLSALLVDVTSDEARRNLMDVLRDHQALSKHTWRVLLSVCRSWAAWCELNNRKWFPAEPEDVRDYLLHLQTRGLTVNTIQQHLCQLNLLHRRSGLPRPGDSNAVSLVMRRIRKENIDAGERVKQALAFERTDFDQVRSLMENSDRCQDIRNLAFLGVAYNTLLRISEIARIRVRDITRTDGGRMLIHIGRTKTLVSAAGEEKALSLGVTKLVERWISVSGVADDRNNYLFCRVKRNGVAAPSAFSQLSTPALHGVFAAAHRLIHGAKDATSAGGSGGSGGSGGSGGSGGSGGSGGSDRERPYACPVESCDRRFSRSDELTRHIRIHTGQKPFQCRICMRNFSRSDHLTTHIRTHTGEKPFACDICGRKFARSDERKRHTKIHLRQKDGRAGGSGGSGGSGGSGGSGGSGGSGGSGPSGQRYLTWSGHSARVGAARDMARAGVPVAEIMQAGGWTTVESVMRYLRNLDSETGAMVRLLEDGD

SEQ ID NO: 31 - protein, 87 aa, synthetic construct
MERPFQCRICMRNFSFHSNLLAHIRTHTGQKPFQCRICMRNFSRKFVLDNHIRTHTGEKPFACDICGRKFAQLGTLRRHTKIHLRGS

SEQ ID NO: 32 - protein, 87 aa, synthetic construct
MERPFQCRICMRNFSFHSNLLAHIRTHTGQKPFQCRICMRNFSRKFVLDNHIRTHTGEKPFACDICGRKFAHASCLSRHTKIHLRGS

SEQ ID NO: 33 - protein, 87 aa, synthetic construct
MERPFQCRICMRNFSNQSGLCRHIRTHTGQKPFQCRICMRNFSFRSGLLQHIRTHTGEKPFACDICGRKFAMRRYLRAHTKIHLRGS

SEQ ID NO: 34 - protein, 87 aa, synthetic construct
MERPFQCRICMRNFSTKRILTDHIRTHTGQKPFQCRICMRNFSFHSGLLAHIRTHTGEKPFACDICGRKFAARRYLVQHTKIHLRGS

SEQ ID NO: 35 - protein, 87 aa, synthetic construct
MERPFQCRICMRNFSRKAHLTMHIRTHTGQKPFQCRICMRNFSAQSNLSRHIRTHTGEKPFACDICGRKFAQLGNLRSHTKIHLRGS

SEQ ID NO: 36 - protein, 116 aa, synthetic construct
MERPYKCPECGKSFSRADNLTEHQRTHTGQKPFQCKTCQRKFSRSDHLKTHTRTHTGEKPYRCKYCDRSFSISSNLQRHVRNIHTGQKPYKCDECGKNFTQSSNLIVHKRIHLRGS

SEQ ID NO: 37 - DNA, 34 bp, unidentified
  source 1..34: mol_type = other DNA
ataaatctgtggaaacgctgctccacagatttat

SEQ ID NO: 38 - DNA, 34 bp, unidentified
  source 1..34: mol_type = other DNA
ctaagattgtgtgaacgctgccacacaatcttag

SEQ ID NO: 39 - DNA, 72 bp, unidentified
  source 1..72: mol_type = other DNA
ggagaattcattgccagctataaatctgtggaaacgctgctccacagatttatagctggcaatgaattctcc

SEQ ID NO: 40 - DNA, 72 bp, unidentified
  source 1..72: mol_type = other DNA
tttctgccaatcttgtgtgctaagattgtgtgaacgctgccacacaatcttagcacacaagattggcagaaa

SEQ ID NO: 41 - protein, 487 aa, synthetic construct
MSNLQTLHQNLSALLANATSDEARKNLMDVFRDRRAFSEATWKTLLSVCRTWAAWCKLNNRKWFPAEPEDVRDYLLHLQVRGLAVNTIQRHLALLNMLHRRSGLPRPGDSSAVSLVMRRIRKENVDAGERVRQALAFERTDFDKVRSLMGNSDRCQDIRNLAFLGVAYNTLLRISEIARIRIKDISRTDGGRMLIHIGRTKTLVSTAGVEKALSLGVTRLVGRWISVSGVAGDPNNYLFCRVRKNGVAAPSATSQLSTDVLRGVFAAAHRLVYGTKDDTSAGGSGGSGGNGGSGGSGGSGGSGGSDRERPFQCHICMRSFSFRSNLLAHIRTHTGQKPFQCHICMRNFSRKFVLDNHIRTHTGEKPFACDICGRKFAQLGTLRRHAKIHLRGSGRADGSGGSGGSGGSGRSGGSGRSGGSGPSGQGYLTWSGHSARVGAARDMARAGVSIAEIMQAGGWTTVESVMSYLRNLDSETGAMVRLLEDGD

SEQ ID NO: 42 - protein, 516 aa, synthetic construct
MSNIQTPHQSLSALLTDATSDVTRKNLADMFRDSQAFSEHTWKMLLSVCRSWAAWCELNNRKWLPVEPEDVRDYLLHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYVLQGIFGAAHRLVYGAKGDTSAGGSGGSGGSGGSGGSGGSGGSGGSDRERPYKCPESGKSFSRADNLTEHQRTQTGQKPFQCKICQRKFSRSEHLKTHTRAHTGEKPYHCKHCDRGFSTSSNLQRHVRNIHTGLKPYKCDECGKNFAQSSNLIVHKRTHLRGSGRTGGSGGSGGSGGSGRSGGSGRSGGSGPSGQKYLAWSGHSARVGAARDMARAGVPIPEIMQAGGWTTVNSVMNYIRNLDSETGAMVRLLEDSD

SEQ ID NO: 43 - protein, 138 aa, synthetic construct
GGSGGSGGNGGSGGSGGSGGSGGSDRERPFQCHICMRSFSFRSNLLAHIRTHTGQKPFQCHICMRNFSRFVLDNHIRTHTGEKPFACDICGRKFAQLGTLRRHAKIHLRGSGRADGSGGSGGSGGSGRSGGSGRSGGS

SEQ ID NO: 44 - protein, 168 aa, synthetic construct
GGSGGSGGSGGSGGSGGSGGSGGSDRERPYKCPESGKSFSRADNLTEHQRTQTGQKPFQCKICQRKFSRSEHLKTHTRAHTGEKPYHCKHCDRGFSTSSNLQRHVRNIHTGLKPYKCDECGKNFAQSSNLIVHKRTHLRGSGRTGGSGGSGGSGGSGRSGGSGRSGGS

SEQ ID NO: 45 - DNA, 34 bp, Homo sapiens
  source 1..34: mol_type = genomic DNA
ttaagattgtgttgtttaatttccacaattttgt

SEQ ID NO: 46 - DNA, 34 bp, Homo sapiens
  source 1..34: mol_type = genomic DNA
atatatctatagatatagatatccacagatatat

SEQ ID NO: 47 - protein, 547 aa, unidentified
MNLTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIANNNGGKQALETVQRLLPVLCQDHGL

SEQ ID NO: 48 - DNA, 18 bp, unidentified
  source 1..18: mol_type = other DNA
tacagaagcgggcaaagg

SEQ ID NO: 49 - protein, 85 aa, unidentified
MERPYACPVESCDRRFSRSDELTRHIRIHTGQKPFQCRICMRNFSRSDHLTTHIRTHTGEKPFACDICGRKFARSDERKRHTKIH

SEQ ID NO: 50 - skipped

SEQ ID NO: 51 - protein, 1165 aa, synthetic construct
MSILLTLHQSLSALLVDATSDEARKNLMDVLRDRQAFSERTWKVLLSVCRTWAAWCKLNNRKWFPAEPEDVRDYLLHLQARGLAVNTILQHLAQLNMLHRRFGLPRPGDSDAVSLVMRRIRRENVDAGERTKQALAFERTDFDQVRALMENSERGQDIRTLALLGVAYNTLLRVSEIARIRIKDISRTDGGRMLIHISRTKTLVSTAGVEKALSLGVTKLVERWISVSGVASDPNNYLFCQVRINGVAVPSATSRLSTDVLRKIFEAAHRLIYGAKDGTSAGGSGGSGGSGGSGGSGGSGGSGGSDRMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIANNNGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGRAGGSGGSGGSGGSGGSGGSGGSGGSGPSGQRYLAWSGHSARVGAARDMARAGVSIAEIMQAGGWTTVESVMNYIRNLDSETGAMVRLLEDGD

SEQ ID NO: 52 - DNA, 80 bp, unidentified
  source 1..80: mol_type = unassigned DNA
tacagaagcgggcaaaggtactcaacccactgcttaagcctcaataaagcttgccttatagacctttgcccgcttctgta

SEQ ID NO: 53 - DNA, 80 bp, unidentified
  source 1..80: mol_type = unassigned DNA
cctttgcccgcttctgtatactcaacccactgcttaagcctcaataaagcttgccttatagatacagaagcgggcaaagg

SEQ ID NO: 54 - DNA, 20 bp, unidentified
  source 1..20: mol_type = unassigned DNA
gggagaattcattgccagct

SEQ ID NO: 55 - DNA, 20 bp, unidentified
  source 1..20: mol_type = unassigned DNA
cacacaagattggcagaaaa

SEQ ID NO: 56 - DNA, 20 bp, unidentified
  source 1..20: mol_type = unassigned DNA
agctggcaatgaattctccc

SEQ ID NO: 57 - DNA, 20 bp, unidentified
  source 1..20: mol_type = unassigned DNA
ttttctgccaatcttgtgtg

SEQ ID NO: 58 - protein, 139 aa, synthetic construct
GGSGGSGGSGGSGGSGGSGGSGGSDRERPFQCRICMRNFSFHSNLLAHIRTHTGQKPFQCRICMRNFSRKFVLDNHIRTHTGEKPFACDICGRKFAQLGTLRRHTKIHLRGSGRAGGSGGSGGSGGSGGSGGSGGSGGS

SEQ ID NO: 59 - protein, 168 aa, synthetic construct
GGSGGSGGSGGSGGSGGSGGSGGSDRERPYKCPECGKSFSRADNLTEHQRTHTGQKPFQCKTCQRKFSRSDHLKTHTRTHTGEKPYRCKYCDRSFSISSNLQRHVRNIHTGQKPYKCDECGKNFTQSSNLIVHKRIHLRGSGRAGGSGGSGGSGGSGGSGGSGGSGGS

SEQ ID NO: 60 - protein, 157 aa, synthetic construct
MDYKDHDGDYKDHDIDYKDDDDKMAPKKKRKVGIHGVPAAMAERPFQCRICMRNFSDRSNLSRHIRTHTGEKPFACDICGRKFAISSNLNSHTKIHTGSQKPFQCRICMRNFSRSDNLARHIRTHTGEKPFACDICGRKFATSGNLTRHTKIHLRGS

SEQ ID NO: 61 - DNA, 12 bp, unidentified
  source 1..12: mol_type = other DNA
gatgaggatgac

SEQ ID NO: 62 - DNA, 68 bp, unidentified
  source 1..68: mol_type = other DNA
gatgaggatgactactcaacccactgcttaagcctcaataaagcttgccttatagagtcatcctcatc

SEQ ID NO: 63 - DNA, 68 bp, unidentified
  source 1..68: mol_type = other DNA
gtcatcctcatctactcaacccactgcttaagcctcaataaagcttgccttatagagatgaggatgac

SEQ ID NO: 64 - protein, 490 aa, synthetic construct
MSILLTLHQSLSALLVDATSDEARKNLMDVLRDRQAFSERTWKVLLSVCRTWAAWCKLNNRKWFPAEPEDVRDYLLHLQARGLAVNTILQHLAQLNMLHRRFGLPRPGDSDAVSLVMRRIRRENVDAGERTKQALAFERTDFDQVRALMENSERGQDIRTLALLGVAYNTLLRVSEIARIRIKDISRTDGGRMLIHISRTKTLVSTAGVEKALSLGVTKLVERWISVSGVASDPNNYLFCQVRINGVAVPSATSRLSTDVLRKIFEAAHRLIYGAKDGTSAGGSGGSGGSGGSGGSGGSGGSGGSDRERPYACPVESCDRRFSRSDELTRHIRIHTGQKPFQCRICMRNFSRSDHLTTHIRTHTGEKPFACDICGRKFARSDERKRHTKIHLRQKDGRAGGSGGSGGSGGSGGSGGSGRSGGSGPSGQRYLAWSGHSARVGAARDMARAGVSIAEIMQAGGWTTVESVMNYIRNLDSETGAMVRLLEDGD

SEQ ID NO: 65 - DNA, 34 bp, unidentified
  source 1..34: mol_type = unassigned DNA
aacccactgcttaagcctcaataaagcttgcctt

SEQ ID NO: 66 - DNA, 64 bp, unidentified
  source 1..64: mol_type = unassigned DNA
agcgtgggcgtactgaacccactgcttaagcctcaataaagcttgccttcagtacgcccacgct

SEQ ID NO: 67 - DNA, 64 bp, unidentified
  source 1..64: mol_type = unassigned DNA
acgcccacgctactcaacccactgcttaagcctcaataaagcttgccttatagagcgtgggcgt

SEQ ID NO: 68 - DNA, 34 bp, unidentified
  source 1..34: mol_type = unassigned DNA
aataggtctgagaacgcccattctcagacgtatt

SEQ ID NO: 69 - DNA, 64 bp, unidentified
  source 1..64: mol_type = other DNA
tgcgtgggcgtgtctaataggtctgagaacgcccattctcagacgtatttactacgcccacgca

SEQ ID NO: 70 - DNA, 64 bp, unidentified
  source 1..64: mol_type = other DNA
acgcccacgcagtctaataggtctgagaacgcccattctcagacgtatttactcgcgtgggcgt

SEQ ID NO: 71 - DNA, 34 bp, unidentified
  source 1..34: mol_type = other DNA
gttaggtctgagaatctgtattctctctcttgaa

SEQ ID NO: 72 - DNA, 64 bp, unidentified
  source 1..64: mol_type = other DNA
tgcgtgggcgtgtctgttaggtctgagaatctgtattctctctcttgaatactacgcccacgca

SEQ ID NO: 73 - DNA, 64 bp, unidentified
  source 1..64: mol_type = other DNA
acgcccacgcagtctgttaggtctgagaatctgtattctctctcttgaatactcgcgtgggcgt

SEQ ID NO: 74 - DNA, 34 bp, unidentified
  source 1..34: mol_type = other DNA
aagaccttagtgatgcccagttgacccaggacgc

SEQ ID NO: 75 - DNA, 64 bp, unidentified
  source 1..64: mol_type = other DNA
tgcgtgggcgtgtctaagaccttagtgatgcccagttgacccaggacgctactacgcccacgca

SEQ ID NO: 76 - DNA, 64 bp, unidentified
  source 1..64: mol_type = other DNA
acgcccacgctactcaagaccttagtgatgcccagttgacccaggacgcatagagcgtgggcgt

SEQ ID NO: 77 - DNA, 70 bp, Homo sapiens
  source 1..70: mol_type = genomic DNA
aagaaatgtaggaaacccacaaatctgtggaaagtaaacgacacactcctaaaaaacaatgggtggaaga

SEQ ID NO: 78 - DNA, 70 bp, Homo sapiens
  source 1..70: mol_type = genomic DNA
atacagaatggaaattgaataaatgcgtggtgaatggaccacagtctctcagcacagaagcaagagcagg

SEQ ID NO: 79 - DNA, 70 bp, Homo sapiens
  source 1..70: mol_type = genomic DNA
tagcaatctatcgattagatatttgtgtggaagaaagcacagaaaatctgatagattttagctaaggaag

SEQ ID NO: 80 - DNA, 70 bp, Homo sapiens
  source 1..70: mol_type = genomic DNA
atagtactagcacctactataaatgggaggatatataggtatacaatgttaaataaaagatatagaagta

SEQ ID NO: 81 - DNA, 70 bp, Homo sapiens
  source 1..70: mol_type = genomic DNA
tatatatgcattctatctgaaaaattgtgtgcttagatatacaaaggtttaccccataataatgaatgtt

SEQ ID NO: 82 - DNA, 70 bp, Homo sapiens
  source 1..70: mol_type = genomic DNA
gaaattaattggatcctaataaaactggtaatggatttgcacccaatcatatctcacatctctaacaggg

SEQ ID NO: 83 - DNA, 70 bp, Homo sapiens
  source 1..70: mol_type = genomic DNA
ccctgttagagatgtgagatatgattgggtgcaaatccattaccagttttattaggatccaattaatttc

SEQ ID NO: 84 - DNA, 70 bp, Homo sapiens
  source 1..70: mol_type = genomic DNA
aataataaaataaagaagctaatattatatagagctagaaccagagatatattttgtaataatgcattgt

SEQ ID NO: 85 - DNA, 64 bp, unidentified
  source 1..64: mol_type = other DNA
acgcccacgctactcctcattacatttaaccaaaattaaatgtaatgagatagagcgtgggcgt

SEQ ID NO: 86 - DNA, 64 bp, unidentified
  source 1..64: mol_type = other DNA
acgcccacgctactcttatattgtgataaccaaaattatcacaatataaatagagcgtgggcgt

SEQ ID NO: 87 - DNA, 64 bp, unidentified
  source 1..64: mol_type = other DNA
acgcccacgctactgccatcttttgttagatttgaataacaaaagatggatagagcgtgggcgt

SEQ ID NO: 88 - DNA, 64 bp, unidentified
  source 1..64: mol_type = other DNA
acgcccacgctactctacacagtgtatattgatttttatacattgtgtaatagagcgtgggcgt

SEQ ID NO: 89 - DNA, 64 bp, unidentified
  source 1..64: mol_type = other DNA
acgcccacgctactgataacctaatataattgtatttatattaggtcagatagagcgtgggcgt

SEQ ID NO: 90 - DNA, 64 bp, unidentified
  source 1..64: mol_type = other DNA
acgcccacgctactgcacactttgttttatgtaggctataccttgataaatagagcgtgggcgt

SEQ ID NO: 91 - protein, 487 aa, synthetic construct
MSNLQTLHQNLSALLANATSDEARKNLMDVFRDRRAFSEATWKTLLSVCRTWAAWCKLNNRKWFPAEPEDVRDYLLHLQVRGLAVNTIQRHLALLNMLHRRSGLPRPGDSSAVSLVMRRIRKENVDAGERVRQALAFERTDFDKVRSLMGNSDRCQDIRNLAFLGVAYNTLLRISEIARIRIKDISRTDGGRMLIHIGRTKTLVSTAGVEKALSLGVTRLVGRWISVSGVAGDPNNYLFCRVRKNGVAAPSATSQLSTDVLRGVFAAAHRLVYGTKDDTSAGGSGGSGGSGGSGGSGGSGGSGGSDRERPFQCRICMRNFSFHSNLLAHIRTHTGQKPFQCRICMRNFSRKFVLDNHIRTHTGEKPFACDICGRKFAQLGTLRRHTKIHLRGSGRAGGSGGSGGSGGSGGSGGSGGSGGSGPSGQGYLTWSGHSARVGAARDMARAGVSIAEIMQAGGWTTVESVMSYLRNLDSETGAMVRLLEDGD

SEQ ID NO: 92 - protein, 516 aa, synthetic construct
MSNIQTPHQSLSALLTDATSDVTRKNLADMFRDSQAFSEHTWKMLLSVCRSWAAWCELNNRKWLPVEPEDVRDYLLHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLVYGAKGDTSAGGSGGSGGSGGSGGSGGSGGSGGSDRERPYKCPECGKSFSRADNLTEHQRTHTGQKPFQCKTCQRKFSRSDHLKTHTRTHTGEKPYRCKYCDRSFSISSNLQRHVRNIHTGQKPYKCDECGKNFTQSSNLIVHKRIHLRGSGRAGGSGGSGGSGGSGGSGGSGGSGGSGPSGQKYLAWSGHSARVGAARDMARAGVPIPEIMQAGGWTTVNSVMNYIRNLDSETGAMVRLLEDSD

SEQ ID NO: 93 - protein, 485 aa, synthetic construct
MSNLQTLHQNLSALLADATSDEARENLMDVFRDRQAFSEATWKTLLSVCRSWAAWCKLNNREWFPAEPEDVRDYLLHLQVRGLAVNTIQRHLALLNMLHRRSGLPRPGDSDAVSLVMRRIRKENVDAGERVKQALAFERTDLDQVRSLMENSDRCKDMRNLAFLGVAYNTLLRISEIARIRVKDISRTDGGRMLVHIGRTKTLVSTAGVEKALSLGVIRLVERWISVSGVAGDPNNYLFCRVRKNGVAVPSATSQLTTDVLRGIFVAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTEESVMSYLRNLDSETGAMVRLLEDGD

SEQ ID NO: 94 - protein, 485 aa, synthetic construct
MSDLQALHQNLSALLVDAASDEVRKHLMIMFRDSQAFSEHTWKMLLSVCRSWAAWCELNNRKWLPAEPEDVRDYLLHLQARGLAVKTIQHHLGSLNMLHRQSGLPRPGDSNAVSLVMRRIRRENVDAGERAKQALAFERTDFDQVRALVENSDRCQDIRNLAFLGVAYNTLLRISEIARIRVGDISHTVGGRMLIHIGRTKTLVSTAGVEKALSLGVTKLVERWISVSGVADDPNNYLFCRVRRYGIAKPSATIQLSTYALQRIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMAEIMQAGGWTTVNSVMNYIRNLDSETGAMVRLLEDDD

SEQ ID NO: 95 - protein, 485 aa, synthetic construct
MSDLLTLHQNLSALLVDAASDVVRKNLMVMFRDSQAFSEHTWKMLLSVCRSWAAWCELNDRKWFPAEPEDVRDYLLHLQARGLAVKTIQHHLGSLNMLHRRSGLPRPGDSNAVSLVIRRIRKENVDAGERAKQALAFERTDLDQVRALMEDSDRCQDIRNLAFLGVAYNTLLRISEIARIRVRDISRTDGGRMLVHIGRTKTLVSTAGVEKALSLGVTELVERWISVSGVADDPNNYLFCRVRRYSVAKPSATSQLSTYALQGIFGAAHRLIYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIAEIMQAGGWTTVNSVMNYIRNLDSETGAMVRLLEDGD

SEQ ID NO: 96 - protein, 485 aa, synthetic construct
MSNLLTLHQNLSALLADATCDEARKNLVDMLRDHQAFSEHTWKMLLSVCQSWAAWCKLNNRKWFPAEPEDVRDYLLHLQARGLAIKTIQHHLGSLNMLHRQSGLPRPGDSNAVSLVMRRIRKENVDAGERAKQALAFERTDFDQVRSLMENSDRCQDIRNLAFLGVAYNTLLRISEIARIRVKDISRTDGGRMLIHIGRTKTLVSTAGVEKALSLGVTKLVERWISVSGVADDPDNYLFCRVRRYGVAKPSATSQLSTYAMQGIFGAAHRLVYGAKGDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKCLAWSGHSARVGAARDMARAGVSLPEIMQAGGWTTVNSVMNYIRNLDSETGAMVRLLEDDD

SEQ ID NO: 97 - protein, 485 aa, synthetic construct; identical to SEQ ID NO: 95

SEQ ID NO: 98 - protein, 485 aa, synthetic construct
MSNLQTLHQNLSALLVDATSGEARRNLMIMFRDSQAFSEHTWKMLLSVCRSWAAWCELNNRKWFPAEPEDVRDYLLHLQARGLAVKTIQHHLGSLNMLHRRAGLPRPGDSDAVSLVMRRIRRENVDAGERAKQALAFERTDFDQVRSLMENSNRCQDIRNLAFLGVAYNTLLRISEIARIRVRYISRTDGGRMLIHIGRTKTLVSTAGVEKALSLGVSKLVERWISVSGVADDPNNYLFCRVRRYGVAKPSATSQLSTYALQQIFAAAHRLIYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVPIAEIMQAGGWTTVNSVMNYIRNLDSETGAMVRLLEDGD

SEQ ID NO: 99 - protein, 485 aa, synthetic construct
MSDLLTLHQDLSALLADATSGEVRKNLMGMFRDHQAFSEHTWKTLLSVCRSWAAWCELNNRKWFPAEPEDVRDYLLHLQARGLAVKTIQHHLGSLDMLHRRSGLPRPGDNNAVSLVMRRIRKGNVDAGERAKQALAFERTDFDQVRALMENSDRCQDMRNLAFLGVAYNTLLRISEIARIRVKDISRTDGGRMLIHIGRTKTLVSTAGVEKALSLGVTRLVERWISVSGVADDPNNYLFCRVRRYGVAKPSATSQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSIAEIMQAGGWTTVNSVMNYIRNLDSETGAMVRLLEDDD

SEQ ID NO: 100 - protein, 485 aa, synthetic construct
MSDLPTIHQTLSALLADVTSDEARRNLMTMFRDHQAFSEHTWKMLLSVCQSWAAWCELNDRKWFPAEPEDVRDYLLHLQARGLAVKTIQHHLGSLNMLHRRSGLPRPGDSNAVSLVMRRIRKENVDAGERAKQALAFERTDFDRVRSLMGNSERCQDIRNLAFLGVAYNTLLRISEIARIRVRDISRTDGGRMLIHIGRTKTLVSTAGVEKALSLGVTRLVERWISVSGVADGPNNYLFCRVRRYGVAKPSATSQLSTYAMQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIAEIMQAGGWTTVNSVMTYIRNLDSETGAMVRLLEDGD

SEQ ID NO: 101 - protein, 485 aa, synthetic construct
MSDLQTLHQNLPALLVDATSDEARKNLVGVFRDSQAFSEATWKTLLSVCRSWAAWCKSNNREWFPAEPEDVRDYLLHLQVRGLAVNTIQRHLALLNMLHRRSGLPRPGDSNAVSLVMRRIRKENVDAGERVKQALAFERTDFDQVRSLMENSDRCQDMRNLAFLGVAYNTLLRMSEIARIRVKDISRTDGGRMLIHIGRTKTLVSSAGVEKALSLGVTGLVERWISVSGVADDPNNYLFCRVRKNGVAAPSATSQLTADVLRGIFAAAHRQVYGARDGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVPIAEIMQAGGWTAVESVMSYLRNLDSETGAMVRLLEDGD

SEQ ID NO: 102 - protein, 485 aa, synthetic construct
MSNLQTLHQNLSALLVGAASDEARKNLMTVFRDRQAFSEHTWKMLLSVCRSWAAWCELNNRKWFPAGPEDVRDYLLHLQARGLAVKTIQHHLGSLNMLHRQSGLPRPGDSNAVPLVVRRIRRENVDAGERAKQALAFERTDFDQVRSLMENSDRCQDIRNLAFLGVAYNTLLRISEIARIRVRDISRTDGGRMLIHIGRTKTLVSTAGVEKALSLGVTKLVERWISVSGVADDPNNYLFCRVRRYGVAKPSATSQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMVRAGVSMAEIMQAGGWTTVNSVMNYIRNLDSEAGAMVRLLEDGD

SEQ ID NO: 103 - protein, 485 aa, synthetic construct
MSNLQTLHQNLSALLVDAASDEARRDLVDVFRDRQAFSEATWKTLLSVFRSWAAWCKLNNRKWFPAEPEGVRDYLLHLQARGLAVNTIQRHLALLNMLHRRSGLPRPGDSNAVSLVMRRIRKENVDAGERVNQALAFERTDFDQVRSLMEDSDRCQDIRNLAFLGVAYNTLLRISEIARIRVKDISRTDGGRMLIHIGRTKTLVSSAGVEKALSLRVTRLVKRWISVSGVADDPNNYLFCRVRKNGAAAPSATSQLSTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 104 - protein, 485 aa, synthetic construct
MSNLQTLHQNLSALLVDAASDEARRDLVDVLRDSQAFSGATWKTLLSVCRSWAAWCKLNNRRWFPAEPEDVRDYLLHLQARGLAVNTIQRHLALLNMLHRRSGLPRPGDSNAVSLVMRRIRKGNVDAGERVKQALAFERTDLDQVRSLMEHSDRCKDIRDLAFLGVAYNTLLRISEIARIRIKDISRTDGGRMLIHIGRTKTLVSTAGVEKALSLGVTRLVERWITVSGVAGDPNNYLFCRVRKNGVAVPSAASQLTTDVLRGIFAAAHRLLHGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGRRYLTWSGHSARVGAARDMARAGVSIAEIMQAGGWTTVESVMSYLRNLDSETGAMVCLLEDGD

SEQ ID NO: 105 - protein, 485 aa, synthetic construct
MSNLQTLHQNLSALLVDATSDEARKNLMGVFRDRQAFSEHTWKTLLSVCRSWAAWCRLNNREWFPAEPEDVRDYLLHLQVRGLTVNTIQRHLALLSMLHRRSGLSRPGDSNAVSLVMRRIRKENVDAGERVKQALAFERTDFDQVRSLMENSDRCKDIRNLAFLGVAYNTLLRISEIARIRIKDISRTDGGRMLIHIGRTKTLVSTAGAEKALSLGVTRLVERWISVSGVADDPNNYLFCRVRKNDVAAPSATSKLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQGYLTWSGHSARVGAARDMARAGVSIAEIMQAGGWTTVESVMGYLRNLDSETGAMVRLLEDGE

SEQ ID NO: 106 - protein, 485 aa, synthetic construct
MSNLQTLHQNLTALLDDATSDEARRNLMDVFRDRQAFSEATWKTLLSVCRSWAAWCKLNNREWFPAEPEDVRDYLLHLQARGLTVNTIQRHLALLNMLHRRSGLPRPGDSNAVSLVMRRVRKENVDAGERVKQALAFERTDFDQVRSLMENSDRCQDMRNLAFLGVAYNTLLRMSEIARIRVKDISRTDGGRMLIHIGRTKTLVCTAGVEKALSLGVTRLVERWISVSGVADDPNNYLFCRVRKNGVAVPSATSQLSTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQGYLTWSGHSARVGAARDMARAGVSIAEIMQAGGWTTVESVMVYLRNLDSETGAMVRLLEDGD

SEQ ID NO: 107 - protein, 485 aa, synthetic construct
MSSLLTLHQDLFALLVDATCDEARKNLTDMFRDRQAFSEHTWKMLLSVCQSWAAWCESNNRKWFPAEPEDVRDYLLHLQARGLAVKTIQHHLGSLNMLHRRSGLPRPGDSDAVSLVMRRIRRENVDAGERAQQALAFERTDFDQVRALMENSDRCQDIRNLAFLGVAYNTLLRISEIARIRVRDISRTDGGRMLIHIGRTKTLVSTAGVEKALSLGVTRLVERWISVSGVADDPNNYLFCRVRRYGVAKPSATSQLSTYALQGIFGAAHRLVYGVKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIAEIMQAGGWTTVNSVMNYIRNLDSETGAMVRLLEDGD

SEQ ID NO: 108 - protein, 485 aa, synthetic construct
MSNLQTLHQNLSASLVDVASDEVRKNLIIMFRDSQAFSEQTWKMLLSVCRSWAAWCELNNRRWFPAEPEDVRDYLLHLQARGLAVKTIQHHLGSLNMLHRRSGLPRPGDSNAVSLVMRRIRKENVDGGERAKQALAFERTDFDQVHSLMGNSDRCQDIRNLAFLGIAYNTLLRISEIARIRVRDISRTDGGRMLIHIGRTKTLVSTAGVEKALSLGVTKLVERWISVSGVADDPSNYLFCRVRRYGVAKPSATSQLSTYALQGIFGAAHRLVYGAKGDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMPEIMQAGGWTKVESVMNYIRNLDSETGAMVRLLEDDD

SEQ ID NO: 109 - protein, 485 aa, synthetic construct
MSNLQTSHQSLTALLVDATSDEARRDLMDVFRDRQAFSEHTWKTLLSVCRSWAAWCKPNNRKWFPAEPEDVRDYLLHLRARGLAVNTIQRHLALLNMLHRRSGQPRPGDSNAVSLVMRRIRKENVEAGERVRQALAFERTDFDQVRSLMENSVRCKDIRNLAFLGVAYNTLLRISEIARIRIKDISRTDGGRMLIHIGRTKTLVSTAGVEKALSLGVTRLVERWISVSGVAGDPNNYLFCRVRKNGVAVPSATSQLTADVLRGIFVAAHRLIYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNMDSETGAMVRLLEDGD

SEQ ID NO: 110 - protein, 485 aa, synthetic construct
MSNLQTLHQSLSALLTDATSDEVRKNLMEMFRDYQAFSEHTWKMLLSVCRSWAAWCGLNNRKWFPAEPEDVRDYLLHLQARGLAVKTIQYHLGSLNVLHRRSGLPRPGDSNAVSLVMRRIRKENVDAGERAKQALAFERTDFDQVRALMGSSDRCQDIRNLAFLGVAYNTLLRISEIARIRVKDISHTDGGRMLIHIGRTKTLVSTAGVEKALSLGVTRLVERWISVSGVADDPNNYLFCRVRRYGVAKPSASSQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIAEIMQAGGWTTVNSVMNYIRNLDSETGAMVRLLEDGD

SEQ ID NO: 111 - protein, 485 aa, synthetic construct
MSDLQTLHQNLSALLADATSGEVRKNLMTMFRDSQAFSEQTWKMLLSVCRSWAAWCELNNRKWFPAEPEDVRDYLLHLQARGLAVKTIQHHLGSLNMLHRRTGLPRPGDGNAVSLVMRRIRRENVDAGERAKQALAFERADFDQVRSLMENSDRCQDIRNLAFLGVAYNTLLRIGEIARIRVKDISHTDGGRMLIHIGRTKTLVSTAGVEKALSLGVTKLVERWISVSGVADDPNNYLFCRVRRYGVAKPSATCQLSTYAMQGIFGAAHRLVHGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMAEVMQAGGWTTVNSVMNYIRNLDSETGAMVRLLEDDD

SEQ ID NO: 112 - protein, 485 aa, synthetic construct
MSNLQTLHQNLTALLADATSDEARKDLMGMFRDHQAFSEHTWKMILSVCRSWAAWCKRSNRKWFPAEPEDVRDYLLHLQARGLAVKTIQHHLGSLNMLHRRAGLPRPGDSNAVSLVMRRIRKENVDAGERAKQALAFERTDFDQVRALMGNSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFAAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIPEIMQAGGWTTVNSVMNYIRNLDSETGAMVRLLEDGD

SEQ ID NO: 113 - protein, 485 aa, synthetic construct
MSDLPTIHQTLSALLADVTSDEARRNLMTMFRDHQAFSEHTWKMLLSVCQSWAAWCELNDRKWFPAEPEDVRDYLLHLQARGLAVKTIQHHLGSLNMLHRRSGLPRPGDSNAVSLVMRRIRKENVDAGERAQQALAFERADLDQVRSLMEGSNRCQDIRNLAFLGVAYNTLLRISEIARIRVRDISRTDGGRMLIHIGRTKTLVSTAGVEKALSLGVTKLVERWISVSGVADDPNNYLFCRVRRYGIAKPSATSQLSTYALQGIFGAAHRLVYGTRDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPPGQKYLAWSGHSARVGAARDMARAGVSIAEIMQAGGWTTVSSVMNYIRNLDSETGAMVRLLEDGE

SEQ ID NO: 114 - protein, 485 aa, synthetic construct
MSDLLTLHQTLSALLVDAASDEARKNLMDMFRDHQAFSEHTWKMLLSACRSWVAWCRLSNRKWFPAEPEDVRDYLLHLQSRGLAVKTIQHHLGSLNMLHRRAGLPRPGDSDAVSLVMRRIRRENVDAGERAKQALAFERTDFDQVRALMENSDRCQDIRNLAFLGVAYNTLLRISEIARIRIRDISRTDGGRMLIHIGRTKTLVSTAGVEKALSLGVTKLVERWISVSGVADDPNNYLFCRVRRYGVAKPSATSQLSTYALQGIFGAAHRLVYGVKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIAEIMQAGGWTTVNSVMNYIRNLDSETGAMVRLLEDGD

SEQ ID NO: 115 - protein, 485 aa, synthetic construct
MSNLQTLHQNLSASLVDATSDGAHKNLMDMLRDHQAFSEHTWKMLLSVCRSWMAWCELNNREWFPAEPEDVRDYLLHLQARGLAVKTIQHHLGSLNMLHRQSGLPRPGDSNAVSLVMRRIRRENVDAGERAKQALAFERTDFDQVRALMENSNRCQDIRNLALLGVAYNTLLRISEIARIRVRDISRTDGGRMLIHIGRTKTLVSTTGVEKALSLGVTKLVERWISVSGVADDPNNYLFCRVRRYGVAKPSATSQLSTYALQGIFGAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQGYLAWSGHSARVGAARDMARAGVPIAEIMQAGGWTTVNSVMNYIRNLDSETGAMVRLLEDGD

SEQ ID NO: 116 - protein, 485 aa, synthetic construct
MSDLLTLHQDLSALLVDATSGEARKNLLDMFRDHQAFSEHTWKMLLSVCRSWAAWCELNNRKWFPAEPEDVRDYLLHLQARGLAVKTIQHHLGSLNMLHRRAGLPRPGDSNAVSLVMRRIRKENVDAGERAKQALAFERTDFDQVRGLMEDSDRCQDIRNLAFLGIAYNTLLRISEIARIRVKDISRTDGGRMLIHIGRTKTLVSTAGVEKALCLGVTKLVERWISVSGVADNPNNYLFCRVRRYGVAEPSTISQLSTYALQGIFGAAHRLVHGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIAEIMQAGGWTTVNSVMNYIRNLDSETGAMVRLLEDDD

SEQ ID NO: 117 - protein, 485 aa, synthetic construct
MSNLQTLHQNLSALLTDATSDEARRNLMGVFRDRQAFSEATWKTLLSVCRSWATWCKLNNRGWFPAEPEDVRDYLLHLQVRGLAVNTIQRHLALLNMLHRRSGLPRPGDSNAVSLVMRRIRKENVDAGERVKQALAFERTDFDRVRSLMENSDRFQDIRNLAFLGVAYNTLLRISEIARIRIKDISRTDGGRMLIHIGRTKTLVSTAGVEKALSLGVTRLVERWISVSGVADDPNNYLFCRVRKNGVAVPSATSQLTTDVLRGIFAAAHRLVYGARDNTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPPGQRYLAWSGHSARVGAAWDMARAGVSIAEIMQAGGWTTVESVMVYLRNLDSETGAMVRLLEDGD

SEQ ID NO: 118 - protein, 485 aa, synthetic construct
MSDLQTLHQSLSALLVDATSDEARKNLMDMFRDSQAFSEHTWKMLLSVCRSWVAWCESNNRKWFPAEPEDVRDYLLHLQTRGLAVKTIQHHLGSLNMLHCQSGLPRPGDSNAVSLVMRRIRKENVDAGERAKQALAFERTDFDQVRLLMESSDRCQDIRNLAFLGIAYNTLLRISEIARIRVRDISRTDGGRMLVHIGRTKTLVSTSGVEKALSLGVTKLVERWISVSGVADDPDNYLFCRVRRYGVAKPSATSQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMPEIMQAGGWTTVNSVMNYIRNLDSETGAMVRLLEDDD

SEQ ID NO: 119 - protein, 485 aa, synthetic construct
MSNLQTSHQNLSALLVNATSNEARKNRVDVFRDRQAFSEATWKTLLSVFRSWAAWCKLNNRKWFPAEPEDVRDYLLHLQVRGLAVNTIQRHLALLNMLHRRSGLPRPGGSNAVSLVMRRIRKENVDAGERVKQALAFERTDFDRVRSLMENSDRCKDIRDLAFLGVAYNTLLRISEIARIRIKDISRTDGGRMLIHIGRTKTLVSTAGVEKALSLGVTGLVERWISVSGVADDPDNYLFCRVRKNGVAVPSATSQLTTDVLRGIFATAHRLVYGARDGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 120 - protein, 485 aa, synthetic construct
MTNLLTLHHSLSALLVDATSDEVRKNLMGMIRDRQAFSEHTWTMLLSVCRSWAAWCELNNRRWFPAEPEDVRDYLLYLQARGLAVKTIQHHLGSLDMLHRQAGLPRPGDGNAVSLVMRRIRRENVDAGERAKQALAFERTDFDQVRSLMENSDHCQDIRNLAFLGVAYNTLLRVSEIARIRVRDISRTDGGRMLIHIGRTKTLVSTTGVEKALSLGVTKLVERWITVSGVADDPNNYLFCRVRRYGVAKPSATSQLSTYALQGIFGAAHRLIYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIAEIMQAGGWTTVSSVMSYIRNLDSEAGAMVRLLEDGD

SEQ ID NO: 121 - protein, 485 aa, synthetic construct
MSNLQNLHQNLSALLVDAPSDEVRKNLMDMFRDSQAFSEHTWKMLLSVCRLWAAWCELNNRKWLPAEPEDVRDYLLHLQARGLAVKTIQHHLGSLNMLHRQSGLPRPGDSNAVSLVMRRIRKENVDVGERAKQALAFERTDFDQVRSLMENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLIYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIPEIMQAGGWTTVASVMNYIRNLDSETGAMVRLLEDGD

SEQ ID NO: 122 - protein, 485 aa, synthetic construct
MSDLQTLHQNLSALLVGATSGEARKNLMTMFRDSRAFSEHTWKMLLSVCRSWAAWCKLNSRRWFPAEPEDVRDYLLHLQARGLAVKTIQHHLGSLNMLHRRAGLPRPGDSNAVSLVMRRIRRENVDAGERAKQALAFERTDFDQIRSLMENSDRCQDIRNLAFLGVAYNTLLRISEIARIRVRDISRTDGGRMLIHIGRTKTLVSTAGVEKALSLGATKLVERWISVSGVADDPNNYLFCRVRRYGVAKPSATSQLSTYALQGIFGAAHRLIYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIAEIMQAGGWTTVNSVMNYIRNLDSETGAMVRLLEDDD

SEQ ID NO: 123 - protein, 485 aa, synthetic construct
MSNLQTLHQNLSALLVDATSDEARENLMDVFRDRQAFSGATWKTLLSVCRSWAAWCRLNNRIWFPAEPEDVRDYLLHLQARGLAVNTIQRHLALLNMLHRRPGLPRPGDGNAVSLVMRRIRKENVDAGERVKQALAFERTDFDQVRSLMETSDRCQDIRNLAFLGAAYNTLLRISEIARIRIKDVSRTDGGRMLIHIGRTKTLVSTAGVEKALSLGVTRLVERWISVSGVADDPDNYLFCRVRKNGVAVPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 124 - protein, 485 aa, synthetic construct
MSNLQTLHQNLSALLVDATSDEARRNLVGVFRDCQAFSDATWKTLLSVCRSWTAWCKLNNRKWFPAEPEDVRDYLLHLQVRGLAVNTIQRHLALLNMLHRRPGLPRPGDSNAVSLVMRRIRKENVDAGERVKQALAFEHTDFDQVRSLMENSDRCKDIRNLAFLGVAYNTLLRISEIARIRVKDISRTDGGRMLVHIGRTKTLVSTAGVEKALSLGVTRLVERWISASGVADDPNNYLFCRVRKNGAAAPSATSQLSTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVPIAEIMQAGGWTTVESVMSYLRNLDSETGAMVRLLEDGD

SEQ ID NO: 125 - protein, 485 aa, synthetic construct
MSNLQTLHQNLSALLVGAASDEARKNLMTVFRDRQAFSEHTWKMLLSVCRSWAAWCGLNNRKWFPVEPEDVRDYLLHLQTRGLAVKTIQHHLGSLNMLHRRAGLPRPGDSNAVSLVMRRIRRENVDAGERAKQALAFERTDFDQVRSLMENSNRCQDIRNLAFLGVAYNTLLRISEIARIRVRDISRTDGGRMLIHIGRTKTLVSTAGVEKALGLGVTKLVERWISASGVADDPNNYLFCRVRRYGVAKPSATSQLSTYALQGIFGAAHRLVHGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMAEVMQAGGWTTVSSVMNYIRNLDSETGAMVRLLEDDD

SEQ ID NO: 126 - protein, 485 aa, synthetic construct
MSNIQTLHQNLSALLVGATSDEARKSLMDVFRDRQAFSEATWKTLLSVCRSWAAWCKSSNRKWFPAEPEDVRDYLLHLQVRGLAVNTIQRHLALLNMLHRRSGLPRPGDSNAVSLVMRRIRKENVGAGERVKQALAFERTDFDQVRSLMESSDRCQDIRNLAFLGAAYNTLLRISEIARIRIKDVSRTDGGRMLIHIGRTKTLVSTAGVEKALSLGVTRLVERWISVSGVADDPDNYLFCRVRKNGVAVPSAASQLTTDVLRGIFAATHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 127 - protein, 485 aa, synthetic construct
MSNLLTLHQNLTELLVDAASDETRKNLMDMFRDSQAFSEYTWKMLMSVCRSWVAWCELNNRRWFPAEPEDVRDYLLYLQARGLAVKTIQHHLGSLNMLHRRAGLSRPGDSNAVSLVMRRVRRENIDAGERAKQALAFERTDFDQVRSLMETSDRCQDIRNLAFLGVAYNTLLRISEIARIRVKDISRTDGGRMLIHIGRTKTLVSTAGVEKALSLGVTKLVERWISASGVAGDPNNYLFCRVRRYGVAKPSATCQLSTYALQGIFGAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMAEVMQAGGWTTVSSVMNYIRNLDSETGAMVRLLEDDD

SEQ ID NO: 128 - protein, 485 aa, synthetic construct
MSNIQTLHQNLSTLLVDATSDEAHKNLMDVFRDRQAFSEATWKTLLSVCRSWTAWCKSNNRKWFPAEPEDVRDYLLHLQVRGLAVNTIQRHLALLNMLHRRSGLPRPGDSDAVSLVMRRIRKENVDAGERVKQALAFERTDLDQVRSLMENSDRCKDIRNLAFLGVAYNTLLRISEIARIRVKDISRTDGGRMLIHIGRTKTLVSTAGVEKALSLGVTRLVERWISVSGVAGDPNNYLFCQVRKNGVAVPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSIAEIMQAGGWTAVESVMSYLRNLDSETGAMVRLLEDGD

SEQ ID NO: 129 - protein, 485 aa, synthetic construct
MSNLQTLHQNLSALLVDATSDEARKNLMGVFRDRQAFSEATWKTLLSVCRSWTAWCKLNNRKWFPAEPEDVRDYLLHLQVRGLAVNTIQRHLALLNMLHRRSRLPRPGDGNAVSLVMRRIRKENVDAGERVKQALAFERADFDQVRSLMENSDRCQDIRNLAFLGVAYNTLLRISEIARIRIKDISRTDGGRMLIHIGRTKTLVSPAGVEKALSLGVTGLVKRWISVSCVADDPNNYLFCRVRKNGVAVPSAASQLTTDVLRGIFVAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRHLTWSGHSARVGAARDMARAGVSIAEIMQAGGWTTEESVMSYLRNLDSETGAMVRLLEDGD

SEQ ID NO: 130 - protein, 485 aa, synthetic construct
MSNIQTLHQNLSALLVGATSDEARKSLMDVFRDRQAFSEATWKTLLSVCRSWAAWCKSNNREWFPAEPEDVRDYLLHLQVRGLAVNTIQRHLALLNMLHRRSGLPRPGDSNAVSLVMRRIRKENVDAGERVNQALPFERTDFDQVRSLMENSDRCLDIRNLAFLGVAYNTLLRISEIARIRIKDISRTDGGRMLIHIDRTKTLVSTAGVEKALSLGVTRLVERWISVSGVADDPNNYLFCRVRKNGVAVPSATSQLSTDVLRGIFVAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVPIAEIMQAGGWTTVESVMSYLRNLDSETGAMVRLLEDGD

SEQ ID NO: 131 - protein, 485 aa, synthetic construct
MSNLQTLHQNLSTLLVNAASDEARGNLMNVFRDRQAFSEATWKTLLSVCRSWAAWCKLNNRKWLPAEPEDARDYLLHLQVRGLAVNTIQRHLALLNMLHRRSGLPRPGDSNAVSLAMRRIRKENVEAGERVKQALPFERTDFDQVRSLMEDSDRCQDIRNLAFLGVAYNTLLRISEIARIRIKDISRTDGGRMLIHIGRTKTLVSTAGVEKALSLGVTRLVEQWISASGVADDPNNYLFCRVRKNGVAVPSATSQLTIDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSIAEIMQAGGWTTVESVMSYLRNLDSETGAMVRLLEDGD

SEQ ID NO: 132 - protein, 485 aa, synthetic construct
MSDLQTLHQNLSALLVDATSDEARRNLVGVFRDRQAFSEATWKTLLSVCRSWAAWCELNDRNWFPAEPGDVRDYLLHLQARSLAVNTIQRHLALLNMLHRRSGLPRPGDSNAVSLVMRRIRKENVDAGERVKQALAFERTDFDHVRSLMENSDRCQDIRNLAFLGVAYNTLLRISEIARIRVKDISRTDGGRMLVHIGRTKTLVSTAGVEKALSLGVTRLVGRWISVSGVAGDPNNYLFCRVRKNGVAVPSAASQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAETMQAGGWTTVESVMSYLRNLDSETGAMVRLLEDGD

SEQ ID NO: 133 - protein, 485 aa, synthetic construct
MSNTQTLHQNLPALLVDATGDEARKDLMGVFRDRQAFSEATWKTLLSVCRSWAAWCKSNNREWFPAEPEDVRDYLLHLQVRGLAVNTVQRHLALLSMLHRRSGLPRPGDSNAVSLVMRRIRKENVDAGERVKQALAFERTDFDKVRSIMEDSDRCKDIRNLAFLGVAYNTLLRISEIARIRVKDISRTDGGRMLIHIGRTKTLVSTTGVEKALSLGVTRLVERWISVSGVAGDPNNYLFCRVRKNGVAAPSATSQLSTDVLRGVFAAAHRLVYGTKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQGYLAWSGHSARVGAARDMARAGVSIAEIMQAGGWTTVESVMSYLRDLDSETGAMVRLLEDGD

SEQ ID NO: 134 - protein, 485 aa, synthetic construct
MSNLQTLHQNLSTLLVDATSDEARKNLMDVFRDRQAFSEATWKTLLSVCRSWAAWCKLNSRKWFPAEPGDVRDYLLHLQVHGLAVNTIQRHLALLNMLHRRAGLPRPGDSNAVSLVMRRIRKENVDAGERVKQALAFERTDFDQVRSLMENSDRCKDIRNLAFLGVAYNTLLRISEIARIRVKDISRTDGGRMLIHIGRTKTLVSTTGVGKALSLGVTRLVERWISVSGVAGDPNNYLFCRVRKNGVAVPSATSQLTADVLRGIFVAAHRLIYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNMDSETGAMVRLLEDGD

SEQ ID NO: 135 - protein, 485 aa, synthetic construct
MSNLQTLHQDLSALLTDATNDEVRKNLMDTFRDSQAFSEHTWKMLLSVCRSWAAWCELNNRKWLPAEPEDVRDYLLHLQARGLAVKTIQHHLGSLNMFHRQSGLPRPGDSNAVSLVMRRIRRENVDAGERAKQALAFERTDFDQVRALVENSDRCQDIRNLAFLGVAYNTLLRISEIARIRVGDISHTVGGRMLIHIGRTKTLVSTAGVEKALSLGVTKLVERWISVSGVADDPDNYLFCRVRRYGVAKPSATCQLSTYALQGIFGAAHRLIYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMAEIMQAGGWTTVNSVMNYIRNLDSETGAMVRLLEDGD

SEQ ID NO: 136 - protein, 485 aa, synthetic construct
MSNLQTLHQNLSALLVDATSDEARRNLMDVFRDRQAFSEHTWKTLLSVCRTWAAWCKLNNRKWFPAEPEDVRDYLLHLQVRGLAVNTIQRHLALLNMLHRRSGLPRPGDSNAVSLVMRRIRKENVDAGERAKQALAFERTDLDLVRSLLENSDRCLDIRNLAFLGVAYNTLLRISEIARIRIKDISRTDGGRMLIHLGRTKTLVSIAGVEKALSLGVTRLVERWISVSGVADDPNSYLFCRVRKNGVAVPSATSQLTTDVLRGIFATAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSIAEIMQAGGWTTVESVMSYLRNLDSETGAMVRLLEDGD

SEQ ID NO: 137 - protein, 485 aa, synthetic construct
MSNPQTLRQKLSALLAEAASYEARKNLMGVFRDRQAFSEATWKTLLSVCRSWAAWCKLNNREWFPAEPEDVRDYLLHLQVRGLAVNTIQRHLALLNMLHRRSGLPRPGDSNAVSLVMRRIRKENVDAGERVKQALPFERTDFDQVRSLMEDSDRCQDIRNLAFLGVAYNTLLRISEIARIRVKDTSRTDGGRMLIHIGRTKTLVSTAGVEKALSLGVTRLVERWTSVSGVADDPNNYLFCRVRKNGVAVPSATSQLTTDVLRKIFVAAHRLIYGAKDNTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPPGQRYLAWSGHSARVGAARDMARAGVSIAEIMQAGGWATVESVMSYLRNLDSETGAMVRLLEDGD

SEQ ID NO: 138 - protein, 485 aa, synthetic construct
MSDLQTLHQNLSALLINAASDEARTNLTGAFRDHQAFSEATWKTLLSVCRSWAAWCKLNNRKWFPAEPEDVRDYLLHLQVRGLAVNTIQRHLALLNMLHRRSGLPRPGDSNAVSLVMRRIRKENVDAGERVNQALAFERTDFDQVRSLMEDSDRFQDIRNLAFLGVAYNTLLRISEVARIRVKDISRTDGGRMLIHIGRTKTLVSTAGVEKALSLGVTRLVERWISVSGVAADPNNYLFCRVRKNGVAVPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 139 - protein, 485 aa, synthetic construct
MSKLLTLHQSLSALLVDATSDETRKNLVIVFRDSQAFSEHTWKMLLSVCRSWASWCKLNNRNWLPAKPEDVRDYLLHLQARGLAVKTIQHHLGSLNMLHRRSGLPRPGDNNAVSLVMRRIRRENVDAGERAKQALAFERTDFDQVRALMENSDRCQDIRNLAFLGVAYNTLLRISEIARIRVRDISHTEGGRMLIHIGRTKTLVSTAGVEKALSLGITKLVERWISVSGVASDPSNYLFCRVRRYGVAKPSASSQLSTYALQGIFGAAHHLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIAEIMQAGGWTTVSSVMNYIRNLDSETGAMVRLLEDGD

SEQ ID NO: 140 - protein, 485 aa, synthetic construct
MSNLQTLHQNLSALLVDATSDEARKNLMDVFRDRQAFSEATWKTLLSVCRSWAAWCKSSNRKWFPAEPEDVRDYLLHLQVRGLAVNTIQRHLALLNMLHRRSGLPRPGDSSAVSLVMRRIRKENVDAGERVRQALAFERTDFDQVRSLMENSVRCLDIRNLAFLGVAYNTLLRISEIARVRIKDISRTDGGRMLIHIGRTKTLVSTTGVEKALSQGVTRLVGRWISVSGVAGDPNNYLFCRVRKNGVAAPSATSQLTTDVLRGIFAAAHRLVHGTKDGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVPIAEIMQAGGWTTVESVMSYLRNLDSETGAMVRLLEDGD

SEQ ID NO: 141 - protein, 485 aa, synthetic construct
MSNLQTLHQNLSALLVDAASDEAHKNLMDVFRDRQAFSEATWKTLLSVCRSWAAWCELNNRRWFPAEPEDVRDYLLHLQVRGLAVNTIQRHLALLNMLHRRSGLPRPGDSNAVSLVMRRIRKENVGAGERVKQALAFKRTDFDRVRSLLENSDRCKDIRNLAFLGVAYNTLLRISEIARIRIKDISRTDGGRMLIHIGRTKTLVSSAGVEKALCLGITRLVERWISASGVADDPNNYLFCRVRKNGVAVPSATSQLTTDVLRGVFAAAHRLVYGAKDGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSIAEIMQAGGWTTEESVMSYLRNLDSETGAMVRLLEDGD

SEQ ID NO: 142 - protein, 485 aa, synthetic construct
MSNLQTLHQNLSALLVDATSDEARKNLMDVLRDRQAFSEATWKTLLSVCRSWTAWCKLNNRKWFPAEPEDVRDYLLHLQVRGLAVNTIQRHLALLNMLHRRSGLPRPGDSNAVSLVMRRIRKENVDAGERVKQALAYERTDFDQVRSLMEDSDRCQDIRNLAFLGVAYNTLLRISEIARIRVKDTSRTDGGRMLIHIGRTKTLVSTTGVEKALSLGVTRLVERWISVSGVADDPNNYLFCRVRKNGVAVPSATSQLSTDVLRGIFVTAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIAEIMQAGGWTTEESVMSYLRNLDSETGAMVRLLEDGD

SEQ ID NO: 143 - protein, 485 aa, synthetic construct
MSNLQTLHQNLTALLDDATSDEARRDLVDVLRDRQAFSEATWKTLLSVCRSWAAWCKSSNRKWFPAEPEDVRDYLLHLQVRGLAVNTIQRHLALLNMLHRRSGLPRPGDSNAVLLVMRRIRKGNVDAGERVKQALAFERTDLDQVRSLMEHSDRCKDIRDLAFLGVAYNTLLRISEIARIRVRDISRTDGGRMLIHIGRTKTLVSTAGVGKALSLGVTRLVERWISVSGAADDPNNYLFCRVRKNGVAVPSATSQLTTDVLRGIFVAAHRLVHGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 144 - protein, 485 aa, synthetic construct
MSKLLTLLQDLSALLVDAPSDEVRENLMDMFRDHQAFSEHTWKMLLSVCRSWAAWRGSNNRKWFPAEPEDVRDYLLHLQARGLAVKTIQHHLGSLNMLHCQSGLPRPGDSNAVSLAMRRIRKENVDAGERAKQALAFERTDFDQVRTLMESSDRCQDIRNLAFLGVAYNTLLRISEIARIRVRDISRTDGGRMLIHIGRTKTLVSTAGVEKALSLGVTKLVERWISVSGVAVDPNNYLFCRVRRYGVAMPSATSQLSTYAMQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMPEIMQAGGWTTVNSVMNYIRNLDSETGAMVRLLEDGD

SEQ ID NO: 145 - protein, 485 aa, synthetic construct
MSNLQTLHQNLSALLVDATSDEARKNLMGVFRDRQAFSEHTWKTLLSVCRSWAAWCRLNNREWFPAEPEDVRDYLLHLQVRGLTVNTIQRHLALLSMLHRRSGLSRPGDSNAVSLVMRRIRKENVDAGERVKQALAFERTDFDQVRSLMENSDRCKDIRNLAFLGVAYNTLLRISEIARIRIKDISRTDGGRMLIHIGRTKTLVSTAGAEKALSLGVTRLVERWISVSGVADDPNNYLFCRVRKNGVAVPSATSLLSTDVLRGIFVAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVPIAEIMQAGGWTAVESVMSYLRNLDSETGAMVRLLEDGD

SEQ ID NO: 146 - protein, 485 aa, synthetic construct
MSDLQTLHQNLSALLVGATSGEARRNLIITFRDSQAFSEHTWKMLLSVCRSWAAWCELHNRKWFPAEPEDVRDYLLHLQARGLAVKTIQHHLGSLDMLHRRSGLARPGDSNAVSLVMRRIRRENVDAGERAKQALAFERTDFDQVRSLMENSNRCQDIRNLAFLGVAYNTLLRISEIARIRVRDISRTDGGRMLIHIGRTKTLVSTAGVEKALSLGVTKLVERWISVSGVADDPNNYLFCRVRRYGIAKPSATSQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSMAEVMQAGGWTTVSSVMNYIRNLDSETGAMVRLLEDGD

SEQ ID NO: 147 - protein, 485 aa, synthetic construct
MSDLQTLHQNLSALLVGATSGEARRNLIITFRDSQAFSEHTWKMLLSVCRSWAAWCELNNRKWFPAEPEDVRDYLLHLQARGLAVKTIQHHLDSLNMLHRRAGLPRPGDSNAVSLVMRRIRRENVDAGERAKQALAFERTDFDQVLSLMENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFAAAHRLVYGTKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIAEIMQAGGWTTVNSVMNYIRNLDSETGAMVRLLEDGD

SEQ ID NO: 148 - protein, 485 aa, synthetic construct
MSNLQTLHQNLSALLADATSDEARRNLTDVFRDRLAFSEATWKTLLSVCRSWAAWCKLNDRKWFPAEPEDVRDYLLHLQVRGLAVNTIQRHLVLLNMLHRRSGLPRPGDSNAVLLVMRRIRKENVDAGERVKQALAFERTDFDQVRSLMENSDRCKDIRNLAFLGVAYNTLLRISEIARIRIKDISRTDGGRMLIHIGRTKTLVSTAGVEKALSLGVTGLVERWISVSGVADDPNNYLFCRVRKNGVAVPSATSQLTTDVLRKIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVGSVMSYLRNLDNETGAMVRLLEDGD

SEQ ID NO: 149 - protein, 485 aa, synthetic construct
MSNIQTLHQNLSALLVDATSDETRKNLMDLLRDHQAFSEHTWKMLLSVCQSWAAWCELNNRKWFPAEPEDVRDYLLHLQARGLAVKTIQHHLGSLNMLHRRAGLPRPGDSNAVSLVMRRIRRENVDAGERAEQALAFERTDFDQVRALMETSDRCQDIRNLAFLGIAYNTLLRISEIARIRVRDISRTDGGRMLIHIGRTKTLVSTAGVEKALSLGVTELVERWISVSGVADNPNNYLFCRVRRYGVAKPSATSQLSTYALQGIFGAAHRLVNGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIPEIMQAGGWTTVNSVMNYIRNLDSETGAMVRLLEDGN

SEQ ID NO: 150 - protein, 485 aa, synthetic construct
MSDLQTLHQNLSALLVDAASDEAHKNLMDVFRDRQAFSEATWKTLLSVCRSWAAWCKLNNRKWFPAEPEDVRDYLLHLQVRGLTVNTIQRHLALLNMLHRRSGLPRPGDSNAVSLVMRRVRKENVDAGERVKQALAFERTDFDQVRSLMENSDRCQDMRNLAFLGVAYNTLLRMSEIARIRVKDISRTDGGRMLIHIGRTKTLVCTAGVEKALSLGVTRLVERWISVSGVADDPNNYLFCRVRKNGVAVPSATSQLSTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQGYLTWSGHSARVGAARDMARAGVSIAEIMQAGGWTTVESVMVYLRNLDSETGAMVRLLEDGD

SEQ ID NO: 151 - protein, 485 aa, synthetic construct
MSNLQTLHQNLSALLVGATSDEARKNLTDVFRDRQAFSGATWKTLQSVCRSWAAWCRSNNRKWFPAEPGDVRDYLLHLQARGLAVNTTQRHLALLNMLHRRSGLPRPGDSNAVSLVMRRIRKENVDAGERVKQALAFERTDFDQVRSLMENSDRCQDIRNLAFLGVAYNTLLRISEIARIRIKDISRTDGGRMLIHIGRTKTLVSTAGVEKALSLGVTRLVERWISVSGVAGDPNNHLFCRVRKNGVAVPSATSQLTTDVLRGIFAAAHNLVYGARDGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSIAEIMQAGGWTTVESVMSYLRNLDSETGAMVRLLEDGD

SEQ ID NO: 152 - protein, 485 aa, synthetic construct
MSDLLTLHQDLSALLVDATCDEVRKNLMAMFRDSQAFSEHTWKMLLSVCQSWAAWCKLNNREWFPAEPEDVRDYLLHLQARGLAVKTIQHHLGSLNMLHRRSGLPRPGDSNAVSLVIRRIRKENVDAGERAKQALAFERTDFDQVRTLMESSDRCQDIRNLAFLGVAYNTLLRISEIARIRVRDISRTDGGRMLIHIGRTKTLVSTAGVEKALSLGVTKLVERWISVSGVAVDPNNYLFCRVRRYGVAMPSATSQLSTYAMQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMPEIMQAGGWTTVNSVMNYIRNLDSETGAMVRLLEDGD

SEQ ID NO: 153 - protein, 485 aa, synthetic construct
MSDLQTLHQNLSALLVGATSGEARRNLIITFRDSQAFSEHTWKMLLTVCRSWAAWCKLNNRKWFPAEPEDVRDYLLHLQARGLAVKTIQHHLGSLNMLHRRAGLPRPGDSNAVSLVMRRIRRENVDAGERAEQALAFERTDFDQVRALMETSDRCQDIRNLAFLGVAYNTLLRISEIARIRVRDISRTDGGRMLIHIGRTKTLVSTAGVEKALSLGVTELVERWISVSGVADNPNNYLFCRVRRYGVAKPSATSQLSTYALQGIFGAAHRLVNGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIAEIMQAGGWTTVNSVMNYIRNLDSETGAMVRLLEDGD

SEQ ID NO: 154 - protein, 485 aa, synthetic construct
MSNLQTLHQNLSALLVDATSDEARKSLMDVFRDRQAFSEATWKTLLSVCRSWAAWCKSNNREWFPAEPEDVRDYLLHLQVRGLAVNTVQRHLALLSMLHRRSGLPRPGDSNAVSLVMRRIRKGNVDAGERVKQALAFERTDLDRVRSLVEDSDRRQDIRNLAFLGVAYNTLLRISEIARIRIKDITRTDGGRMLIHIGRTKTLVSTAGVEKALSLGVTRLVERWISVSGVADDPNNYLFCRVRKNGVAVPSATSQLTTDVLRGIFVAAHRLVYGAQDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQGYLTWSGHSARVGAARDMARAGVSIAEIMQAGGWTTVESVMSYLRNLDSETGAMVRLLEDGD

SEQ ID NO: 155 - protein, 485 aa, synthetic construct
MSNLQTLHQNLSALLVDAASDEARRDLVDVLRDSQAFSGATWKTLLSVCRSWAAWCKLNNRRWFPAEPEDVRDYLLHLQARGLAVNTIQRHLALLNMLHRRSGLPRPGDSNAVSLVMRRVRKENVDAGERVKQALAFERTDFDQVRSLMENSDRCKDIRNLAFLGVAYNTLLRISEIARIRVRDISRTDGGRMLIHIGRTKTLVSTAGVEKALSLGVTRLVGRWISVSGVAGDPNNYLFCRVRKNGVAVPSATSQLTTDVLRGIFAAAHRLVHGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSDQRYLAWSGHSARVGAARDMARAGVSIAEIMQAGGWTTVESVMSYLRNLDSETGAMVRLLEDGD

SEQ ID NO: 156 - protein, 485 aa, synthetic construct
MSNLQTLHQTLSALLVDATSDEARKNLMNMFRDYQAFSEHTWKMLLTVCRSWAAWCELNNREWFPAEPEDVRDYLLHLQARGLAVKTIQHHLGSLNMLHRRSGLPRPGDSNAVSLVMRRIRRENVDAGECAKQALAFERTDFDQVRSLMVNSDRRQDIRNLAFLGVAYNTLLRISEIARIRVRDISRTDGGRMLIHIGRTKTLVSTAGVEKALSLGVTKLVERWISVSGVADDPDNYLFCRVRRYGVAKPSATSQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARTGVPIPEIMQAGGWTTVNSVMNYIRNLDSETGAMVRLLEDGD

SEQ ID NO: 157 - protein, 485 aa, synthetic construct
MSNLQTLHQSLSALLADATSDEARKNLIDVFRDRQAFSEATWKTLLSVCRSWAAWCKLNDRKWFPAAPEDVRDYLLHLQVRGLVVNTIQRHLALLNMLHRRSGLSRPGDSNAVSLVMRRIRKENVDAGERVRQALAFERTDFDQVRSLMENSVRCKDIRNLAFLGVAYNTLLRISEIARIRTKDISRTDGGRMLIHIGRTKTLVSTAGVEKALSLGVTRLVERWISVSDVAGDPNNYLFCRVRKNGVAVPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQGYLTWSGHSARVGAARDMARAGVSIAEIMQAGGWTTVESVMSYLRNLDSETGAMVRLLEDGG

SEQ ID NO: 158 - protein, 485 aa, synthetic construct
MSNLQTLHQNLSALLADATSNEARKNLADVFRDRQAFSEATWKTLLSVCRSWAAWCKSNNREWFPAEPGDVRDYLLHLQVRGLAVNTIQRHLALLSMLHRRSGLPRPGDSNAVSLVMRRIRKENVDAGERVKQALAFERTDFDQVRSLMENSDRCQDIRNLAFLGVAYNTLLRLSEIARIRIKDISRTDGGRMLIHIGRTKTLVSTAGVEKALSLGVTRLVERWISVSGVADDPNNYLFCRVRKNGVAVPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVCLLEGGD

SEQ ID NO: 159 - protein, 485 aa, synthetic construct
MSNLQTLHQNLSALLVDAMSNEARKNLADVFRDRQAFSEATWKTLLSVCRSWAAWCKSNNREWFPAEPGDVRDYLLHLQVRGLAVNTIQRHLALLSMLHRRSGLPRPGDSNAVSLVMRRIRKENVDAGERVKQALAFERTDFDQVRSLMENSDRCQDIRNLAFLGVAYNTLLRLSEIARIRIKDISRTDGGRMLIHIGRTKTLVSTAGVEKALSLGVTRLVERWISVSGVADDPNNYLFCRVRKNGVAVPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVCLLEGGD

SEQ ID NO: 160 - protein, 485 aa, synthetic construct
MSNLQTLHQNLSALLVDATGYEARRNLMDVFRDRQAFSEATWKTLLSVCRSWAAWCKSSNRKWFPAEPEDVRDYLLHLQVRGLAVNTIQRHLALLNMLHRRSGLPRPGDSNAVSLVMRRIRKENVDAGERVKQALAFERTDFDQVRSLMENSDRCQDIRNLAFLGVAYNTLLRISEIARIRVGDISRTDGGRMLVHIGRTKTLVSTAGVEKALSLGVTGLVERWISVSGVADDPNNYLFCRVRKNGVAAPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQGYLTWSGHSARVGAARDMARAGVSIAEIMQAGGWTTVESVMSYLRNLDSETGAMVRLLEDGD

SEQ ID NO: 161 - protein, 485 aa, synthetic construct
MSNLQTLHQNLSALLTDATSVEARKNLIDVFRDRQAFSEATWKTLLSVCRTWAAWCKLNNRIWFPAEPEDVRDYLLHLQVRGLAVNTIQRHLALLNMLHRRSGLPRPGDSSAVSLVMRRIRKENVDAGERVKQALAFERTDFDQVRSLMGNSDRCKDIRNLAFLGVAYNTLLRISEIARIRIKDISRTDGGRMLIHIGRTKTLVSTAGVEKALSLGVTGLVERWTSVSGAAGDPNNYLFCRVRKNGVAVPSATSLLSTDVLRGIFVAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVPIAEIMQAGGWTTVESVMSYLRNLDSETGAMVRLLEDGD

SEQ ID NO: 162 - protein, 485 aa, synthetic construct
MSNLQTLHQNLSALLADATSDEARKNLIDVFRDRQAFSEATWKTLLSVCRSGAAWCKLNNRKWFPAEPEDVRDYLLHLQVRGLAVSTIQRHLALLNMLHRRSGLPRPGDSNAVSLVMRRIRKENVDAGERVRQALAFERTDFDKVRSIMEDSDRCKDIRNLAFLGVAYNTLLRISEIARIRVRDISRTDGGRMLIHIGRTKTLVSTAGVEKALSLGVTRLVERWISASGVADDPNNYLFCRVRKNGVAVPSATSQLSTDVLRGIFAAAHRLVYGAKGGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSIAEIMQAGGWTTVESVMSYLRNLDSETGAMVRLLEDGD

SEQ ID NO: 163 - protein, 485 aa, synthetic construct
MSNIQTLHQNLSALLVDAMSNEARKNLADVFRDRQAFSEATWKTLLSVCRSWAAWCKSNNREWFPAEPGDVRDYLLHLQVRGLAVNTIQRHLALLSMLHRRSGLPRPGDSNAVSLVMRRIRKENVDAGERVKQALAFERTDFDQVRSLMENSDRCQDIRNLAFLGVAYNTLLRLSEIARIRIKDISRTDGGRMLIHIGRTKTLVSTAGVEKALSLGVTRLVERWISVSGVADDPNNYLFCRVRKNGVAVPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVCLLEGGD

SEQ ID NO: 164 - protein, 485 aa, synthetic construct
MSNLQTLHQNSSALLVDATSDEARKNLMDVFRDRQAFSEATWKTLLSVCRTWAAWCKLNNREWFPAEPEDVRDYLLHLQVRGLAVNTIQRHLALLNMLHRRSGLPRPGDSNAVSLVVRRIRKENVDAGERVKQALAFERTDLDQVRSLMENSDRCQDIRNLAFLGVAYNTLLRISEIARIRIKDISRTDGGRMLIHIGRTKTLVSSAGVEKALSLGVTRLVERWTSVSGVADDPNNYLFCRVRKNGVAVPSATSQLSTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRHLTWSGHSARVGAARDMARAGVSIAEIMQAGGWTTEESVMSYLRNLDSETGAMVRLLEDGD

SEQ ID NO: 165 - protein, 485 aa, synthetic construct
MSNLQTLHQNLSALLADATSDEARENLMGVSRDRQAFSEATWKTLLSVCRSWAAWCKLNNQRRFPAGPEDVRDYLLHLQVRGLAVNTIQRHLALLNMLHRRSGLPRPGDGNAVSLVMRRIRKENVDAGERVRQALAFERTDFDQVRSLMGNSDRCKDIRNLAFLGVAYNTLLRISEIARIRIKDISRTDGGRMLIHIGRTKTLVSTAGVEKALSLGVTRLVGRWISVSGVAGDPNNYLFCRVRKNGVAVPSATSQLSTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVPIAEIMQAGGWTTVESVMSYLRNLDSETGAMVRLLEDGD

SEQ ID NO: 166 - protein, 485 aa, synthetic construct
MSNSQTLHQNLSALLTDATSDEARRNLMDVLRDRQAFSEATWKTLLSVCRSWAAWCKLNNRKWFPAEPEDVRDYLLHLQVRGLAVNTIQRHLALLNMLHRRSGLPRPGDSNAVSLAMRRIRKENVDAGERVKQALAFECTDFDQVRSLMENSDRCQDIRTLAFLGVAYNTLLRISEIARIRIKDISRTDGGRMLIHIGRTKTLVSTAGVEKALSLGVTRLVERWISVSGVADDPNNYLFCRVRKNGVAAPSAASQLTTDVLRGIFAAAHRLIYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 167 - protein, 485 aa, synthetic construct
MSNLQTLHQNLSALLVDATSGGARKNLMGVFRDRQAFSEATWKTLLSVCRSWTAWCRLNNREWFPAEPEGVRDYLLHLQVRGLAVNTIQRHLALLNMLHRRSGLPRPGDSNAVSLVMRRIRKENVDAGEHVKQALAFERTDFDQVRSLMENSDRCQDIRNLAFLGVAYNTLLRISEIARIRIKDISRTDGGRMLIHIGRTKTLVSTAGVEKALSLGVTRLVERWISVSGVAGDPNNYLFCRVRKNGVAVPSATSQLTTDVLRGVFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARARVSIAEIMQAGGWTTVESVMSYLRNLDSETGVMVRLLEDGD

SEQ ID NO: 168 - protein, 485 aa, synthetic construct
MSNLQTLHQNLSALLADATSDETRKNLIDVFRDRQAFSEATWKTLLSVCRSWAAWCKLNNRKWFPAEPEDVRDYLLHLQVRGLAVNTIQRHLALLNMLHRRSGLPRPGDSNAVSLVMRRIRKENVDAGERVKQAQAFERTDFDQVRSLMENSVRCLDIRNLAFLGVAYNTLLRISEIARIRIKDISRTDGGRMLIHIGRTKTLVSTAGVEKALSLGVTRLVERWISVSGVADDPNNYLFCRVRKNGVAVPSATSQLTADVLRGIFAAAHRLIYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVCLLEEGD

SEQ ID NO: 169 - protein, 485 aa, synthetic construct
MSNLQTLHQNLSALLTDATSDEARKNLMGVFRDRQAFSEATWKTLLSVCRSWAAWGKLNNRRWFPAEPEDVRDYLLHLQARGLAVNTVQRHLALLSMLHRRSGLPRPGDSNAVSLVMRRIRKENVDAGERVKQALAFERTDFDQVRSLMENSDRCKDIRNLALLGVAYNTLLRISEIARIRIRDISRTDGGRMLIHIGRTKTLVSTAGAEKALSLGVTRLVERWISVSGVAGDPNNYLFCRVRKNGVAAPSATSQLSTDFLRKIFAAAHRLVYGARGDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMHAGGWTTVESVMSYLRNLDSETGAMVRLLEDGD

SEQ ID NO: 170 - protein, 485 aa, synthetic construct
MSDLQTLHQSLSASLANAPSDEVRKNLIDMFRDYQAFSEHTWRMLLSVCRSWATWCRLNNRKWFPAEPEDVRDYLLHLQARGLAVKTIQHHLGSLDMLHRQSGLPRPGDSNAVSLVMRRIRRENVDAGERAKQALAFERTDFDRVRALMESSDRCQDIRNLAFLGVAYNTLLRISEIARIRVKDISRTDGGRMLIHIGRTKTLVSTAGEVKALSLGVTKLVERWISVSGVADDPNNYLFCRVRRYGVAKPSATSQLSTYALQGIFGAAHRLIHGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLTWSGHSARVGAARDMARAGVSMPEIMQAGGWTTVSSVMNYIRNLDSETGAMVRLLEDGD

SEQ ID NO: 171 - protein, 485 aa, synthetic construct
MSNLQTLHQDLSALLTDATNDEVRKNLIDMFRDHQAFSEHTWKTLLSVCRSWAAWCGLNNRKWFPVEPEDVRDYLLHLQTRGLAVKTIQHHLGSLNMLHRRAGLPRPGDSNAVPLVMRRIRRENVDAGERAKQALAFERTDFDQVRLLMESSDRCQDIRNLAFLGIAYNTLLRISEIARIRVRDISRTDGGRMLVHIGRTKTLVSTSGVEKALSLGVTKLVERWISVSGVADDPDNYLFCRVRRYGVAKPSATSQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMPEIMQAGGWTTVNSVMNYIRNLDSETGAMVRLLEDGD

SEQ ID NO: 172 - protein, 485 aa, synthetic construct
MSDLQTLHQNLFALLVGAASDEARRNLIDVFRDRQAFSEATWKTLLSVCRSWAAWCKLNNRKWFPAEPEDVRDYLLHLQVRGLAVNTIQRHLALLNMLHRRSGLPRPGDSNAVSLVMRRIRKGNVDAGERVKQALAFERADLDQVHSLMENSDRCQDIRNLAFLGVAYNTLLRISEVARIRVKDISRTDGGRMLIHIGRTKTLVSTAGVEKALSLGVTRLVERWISVSGVADDPNNYLFCRVRKNGVAVPSATSQLTTDVLRGIFATAHRLVYGARDGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVHLLEDGD

SEQ ID NO: 173 - protein, 485 aa, synthetic construct; identical to SEQ ID NO: 111

SEQ ID NO: 174 - protein, 485 aa, synthetic construct
MSDLQTLHQSLSALLVDATSDEARKNLAVMFRDHQAFSEHTWKMLLSVCRSWAAWRELNSRKWFPAEPEDVRDYLLHLQARGLAVKTIQHHLGSLNMLHRQSGLPRPGDSNAVSLVMRRIRRENVDAGERAKQALAFERTDFDQVRSLVENSDRCQDIRNLAFLGVAYNTLLRISEIARIRVRDISRTDGGRMLIHIGRTKTLVSTAGVEKALSLGVSKLVERWISVSGVADDPNNYLFCRVRRYGVAKPSATSQLSTYALQGIFGAAHRLVYGAKGDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIAEIMQAGGWTTVSSVMNYIRNLDSETGAMVRLLEDDD

SEQ ID NO: 175 - protein, 485 aa, synthetic construct
MSDLQTLHQDLSALLVDATSDEIRKNLTDMFRDSQAFSEHTWKMLLSVCRSWAAWCELNNRKWFPAEPEDVRDFLLHLQARGLAVKTIQHHLGSLNMLHRRSGLPRPGDSNAVSLVMRRIRRENVDAGERARQALAFERPDFDQVRSLLENSDRRQDIRNLAFLGVAYNTLLRISEIARIRVRDISRTDGGRMLIHIGRTKTLVSTAGVEKALSLGVTKLVERWISVSGVADDPDNYLFCRVRRYGVAKPSATCQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMAEIMQAGGWTTVNSVMNYIRNLDSETGAMVRLLEDGD

SEQ ID NO: 176 - protein, 485 aa, synthetic construct
MSNIQTLHQNLSALLVGATSDEARKSLMDVFRDRQAFSEATWKTLLSVCRSWAAWCKSNNREWFPAEPEDVRDYLLHLQVRGLAVNTIQRHLALLNMLHRRSGLPRPGDSNAVSLAMRRIRKENVDAGERVKQALAFERTNFDQVRSLMENSDRFQDIRNLAFLGVAYNTLLRISEIARIRIKDISRTDGGRMLINIGRTKSLVSTAGVEKALSLGVTRLVERWISVSGVADDPNNYLFCRVRKNGVAAPSATSQLSTDFLRKIFAAAHHMIYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRHLTWSGHSARVGAARDMARAGVSIAEIMQAGGWTTVESVMGYLRNLDSETGAMVRLLEDGD

SEQ ID NO: 177 - protein, 485 aa, synthetic construct
MSNLQTIHQNLSALLVDATSDEARKNLMDVFRDRQAFSEATWKTLLSVCRSWAAWCELNDRNWFPAEPGDVRDYLLHLQARSLAVNTIQRHLALLNMLHRRSGLPRPGDDNAVSLVMRRIRKENVDAGERVKQALAFERTDFDQVRSLMENSDRCKDIRNLAFLGVAYNTLLRISEIARIRIKDISRTDGGRMLIHIGRTKTLVSTAGVEKALSLGVTRLVEQWISASGVADDPNNYLFCRVRKNGVAVPSATSQLTIDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSIAEIMQAGGWTTVESVMSYLRNLDSETGAMVRLLEDGD

SEQ ID NO: 178 - protein, 485 aa, synthetic construct
MSNLQTLHQNLSALLVDATSDEARKNLMDAFRDRQAFSEHTWKTLLSVCRSWAAWCELNNREWFPAEPEGVRDYLLHLQARGLAVNTIQRHLALLNMLHRRSGLPRPGDSNAVSLVMRRIRKENVDAGERVKQALAFERSDFGRVRSLMENSDRCQDIRNLAFLGVAYNTLLRISEIARIRVKDISRTDGGRMLIHIGRTKTLVSTAGVEKALSLGVTRLVERWISVSGVAGDPNNYLFCRVRKNGVAVPSANSQLTTDVLRGVFAAAHRLVYGARDGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLAWSGHSARVGAARDMARAGVSIAEIMQAGGWTTVESVMSYLRNLDSETGAMVRLLEDGD

SEQ ID NO: 179 - protein, 485 aa, synthetic construct
MSNLQTLHQNLTALLVDATSDEAHKNLIGVFRDRQAFSEATWKTLLSVCRSWAAWCELNNRRWFPAEPEDVRDYLLHLQVRGLAVNTIQRHLALLNMLHRRSGLPRPGDSDAVSLVMRRIRKENVDAGERVKQALAFERTDLDQVRSLMENSDRCKDMRNLAFLGVAYNTLLRISEIARIRVKDISRTDGGRMLVHIGRTKTLVSTAGVEKALSLGVTGLVERWISVSGVADDPDNYLFCRVRKNGVAVPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQGYLAWSGHSARVGAARDMARAGVSIAEIMQAGGWITVESVMSYIRNLDSETGVMVRLLEDGD

SEQ ID NO: 180 - protein, 485 aa, synthetic construct
MSNLQTLHQNLSALLVDAASDVVRKNLMVMLRDHQAFSEYTWKMLLSACRSWAAWCKLNNRKWFPAEPEDVRDYLLHLQARGLAVKTIQHHLGSLNMLHRRSGLPRPGDSNAVSLVMRRIRRENVDAGERAKQALAFERTDFDQVRSLMETSDHCQDIRNLAFLGVAYNTLLRISEIARIRIKDISRTDGGRMLIHIGRTKTLVSTAGVEKALSLGVTKLVERWISVSGVADDPDNYLFCRVRRYGIAKPSATSQLSTYAMQGIFGAAHRLIYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIAEIMQAGGWTTVSSVMSYIRNLDSEAGAMVRLLEDDD

SEQ ID NO: 181 - protein, 485 aa, synthetic construct; identical to SEQ ID NO: 128

SEQ ID NO: 182 - protein, 485 aa, synthetic construct
MSNLLTLHQILSVLLVDAACDEVRKNLMEMFRDRKAFSEHTWKMLMSVCRSWVAWCELNNRRWFPAEPEDVRDYLLYLQARGLAVKTIQHHLGSLNMLHRRAGLPRPGDSNAVSLVMRRIRKGNVDAGERAKQALAFERTDFDQVRALMENSDRCQDMRNLAFLGVAYNTLLRIGEIARIRVRDISRTDGGRMLIHIGRTKTLVSTAGVEKALSLGVTKLVERWISVSGVANDPNNYLFCRVRRYSVAKPSATSQLSTYALQGIFGAAHRLVYGVKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIPEIMQAGGWTTVASVMNYIRNLDSETGAMVRLLEDGD

SEQ ID NO: 183 - protein, 485 aa, synthetic construct
MSDLLTLHQNLSALLADATCDEARKNLVDMLRDHQAFSEHTWKMLLSVCRSWAAWCKLNNRKWFPAEPEDVRDYLLHLQARGLAVKTIQHHLGSLNMLHRQSGLPRPGDSNAVSLVMRRIRRENVDAGERAKQALAFERTDFDQVRSLMENSNRCQDIRNLAFLGVAYNTLLRISEIARIRVRDTSRTDGGRMLVHIGRTKTLVSTAGVEKALSLGVTKLVERWISVSGVADDPNNYLFCRVRRYGVAKPSTTSQLSTYALQGIFGAAHRLIYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIPEIMQAGGWTTVNSVMNYIRNLDSETGAMVRLLEDGD

SEQ ID NO: 184 - protein, 485 aa, synthetic construct
MSNLQTLHQNLSALLVDAASDEARRDLVDVLRDSQAFSGATWKTLLSVCRSWAAWCKLNNRRWFPAEPEDVRDYLLHLQARGLAVNTIQRHLALLNMLHRRSGLPRPGDSNAVSLVMRRIRRENVDAGERAKQALAFERTDFDQVRTLMEDSDRCQDIRNLAFLGVAYNTLLRISEIARIRIRDISRTDGGRMLIHIGRTKALVSTAGVEKALSLGVTRLVERWISVSGVAADPNNYLFCRVRKNGVAVPSATSQLTTDVLRGIFAAAHRLLHGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGRRYLTWSGHSARVGAARDMARAGVSIAEIMQAGGWTTVESVMSYLRNLDSETGAMVHLLEDGE

SEQ ID NO: 185 - protein, 485 aa, synthetic construct
MSNLQTLHQNLSALLDDAASDEVRRNLMGMFRDSQAFSEHTWKMLQSICRSWAAWCESNSRKWFPAEPEDVRDYLLHLQARGLAVKTIQHHLGSLNMLHRQSGLPRPGDSNAVSLVMRRIRRENVDAGERAKQALAFERTDFDHVRSLMENSDRRQDIRNLAFLGVAYNTLLRISEIARIRVRDISRTDGGRMLIHIGRTKTLVSTAGVEKALSLGVTELVERWITVSGVADNPDNYLFCRVRRYGVAKPSATSQLSTYAMQGIFGAAHRLVYGAKGDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKCLAWSGHSARVGAARDMARAGVSLPEIMQAGGWTTVNSVMNYIRNLDSEAGAMVRLLEDGD

SEQ ID NO: 186 - protein, 485 aa, synthetic construct
MSNLQTLHQDLSALLADAASDEARKNLIDVFRDRQAFSEATWKTLLSVCRTWAAWCKSNNRKWFPAEPEDVRDYLLHLQVRGLAVNTIQRHLALLNMLHRRSGLPRPGDSNAVSLVMRRIRKENVGAGERVKQALAFERTDFDQVRSLMEDSDRCRDIRTLAFLGVAYNTLLRISEIARIRIEDISRTDGGRILIHIGRTKTLVSTAGVEKALSLGVTGLVERWISVSDVAGDPNNYLFCRVRKNGVAVPSATSQLSTDVLRGIFAAAHCLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSDQRYLAWSGHSARVGAARDMARAGVPIAEIMQAGGWTTVESVMSYLRNLDSETGAMVRLLEDGD

SEQ ID NO: 187 - protein, 485 aa, synthetic construct
MSNLQTLHQNLSALLVDATSDEARKNLIGVFRDRQAFSEATWKTLLSVCRSWTAWCELNNREWFPAEPEDVRDYLLHLQVRGLAVNTIQRHLALLNMLHRRSGQPRPGDSNAVSLVMRRIRKENVEAGERVRQALAFKRTDFDRVRSLLENSNRCKDIRNLAFLGVAYNTLLRVSEIARIRIKDISRTDGGRMLIHIGRTKTLVSTAGTEKALSLGVIRLVERWISVSGVAGDPNNYLFCRVRKNGVAVPSATSQLTTDVLRGIFVAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWATVESVMSYLRNLDSETGAMVRLLEDGG

SEQ ID NO: 188 - protein, 485 aa, synthetic construct
MSNLQTLHQNLSALLVGATSDEARKDLMDVFRDRQAFSEATWKTLLSVCRSWAAWCKLNNRKWFPAEPEDVRDYLLHLQVRGLAVSTIQRHLALLNMLHRRSGLPRPGDSNAVSLVMRRIRKENVDAGERVKQALAFERTDFDQVRSLMENSDRCQDIRNLAFLGVAYNTLLRLSEIARIRIKDISRTDGGRMLIHIGRTKTLVSTAGVEKALSLGVTRLVERWISASGVADDPNNYLFCRVRKNGVAVPSATSQLSTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRHLTWSGHSARVGAARDMARAGVSIAEIMQAGGWTTVESVMGYLRNLDSETGAMVRLLEDGD

SEQ ID NO: 189 - protein, 485 aa, synthetic construct
MSNLQTLHQNLSASLVDVASDEVRKNLIIMFRDSQAFSEHTWKMLLSVCRSWAAWCELNNRKWFPAEPEDVRDYLLHLQARGLAVKTVQHHLGSLNMLHRRAGLPRPGDSNAVSLVMRRIRRENVDAGERAKQALAFERTDLDRVRSLVEDSDRRQDIRNLAFLGVAYNTLLRIGEIARIRVRDISRTDGGRMLIHIGRTKTLVSTAGVEKALSLGVTKLVERWISVSNVADDPNNYLFCRVRRYGVAKPSATSQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSIPEIMQAGGWTTVSSVTNYIRNLDSETGAMVRLLEDGD

SEQ ID NO: 190 - protein, 485 aa, synthetic construct
MSNLQTLHQNLSALLVDATSGGARKNLMGVFRDSQAFSEHTWKMLQSICRSWAAWCESNNRKWFPAEPEDVRDYLLHLQVRGLAVNTIQRHLALLNMLHRRSGLPRPGDSNAVSLVMRRIRKGNVDAGERAKQALAFERTDFDQVRALMENSDRCQDMRNLAFLGVAYNTLLRISEIARIRVKDISRTDGGRMLIHIGRTKTLVSTAGVEKALSLGVTRLVERWISVSGVAGDPDNYLFCRVRKNGVAAPSATSQLSTDVLRGIFAAAHRLVYGARDNTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLIWSGHSARVGAARDMARAGVSIAEIMQAGGWTTVESVMVYLRDLDSETGAMVRLLEDGE

SEQ ID NO: 191 - protein, 485 aa, synthetic construct
MSNLQTLHHSLSALLVNATSDEARKNLADVFRDRQAFSEATWKTLLSVCRSWTAWCRSNNRKWFPAEPEDVRDYLLHLQVRGLAVNTIQRHLALLNMLHRRSGLPRPGDSNAVSLVMRRIRKENVGAGERVKQALAFERTDFDQVRSLMENSYRCQDIRNLAFLGVAYNTLLRISEIARIRVKDTSRTDGGRMLIHIGRTKTLVSTAGVEKALSLGVTRLVERWIFVSGVADDPNNYLFCRVRKNGVAVPSATSQLTTDVLRGIFEAAHRLVYGARDGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIPEIMQAGGWTTVESVMSYLRNLDSETGAMVRLLEDGD

SEQ ID NO: 192 - protein, 485 aa, synthetic construct
MSNLQTLHQNLSALLVGAASDEARKNLMTVFRDRQAFSEHTWKMLLSVCRSWAAWCELNNRKWFPAGPEDVRDYLLHLQARGLAVKTIQHHLGSLNMLHRQSGLPRPGDSNAVSLVMRRIRRENVDAGERAKQALAFERSDLDQVRAHMEDSDRPQDIRNLAFLGIAYNTLLRISEIARIRVKDISRTDGGRMLIHIGRTKTLVSTAGVEKALSLGVTKLVERWISVSGVADDPNNYLFCRIRRYGVAKPSATSQLSTYALQGIFGAAHGLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGRKYLAWSGHSARVGAARDMARAGVSMAEIMQAGGWTTVNSVMNYIRNLDSETGAMVRLLEDDD

SEQ ID NO: 193 - protein, 485 aa, synthetic construct
MSSLQTLLQDLSALLVDAPSDEVRENLMDMFRDHQAFSEHTWKMLLSVCQSWAAWCKLNNRKWFPAEPEDVRDYLLHLQARGLAVKTIQHHLGSLNMLHRWAGLPRPGDSNAVSLVMRRIRKENIDTGERAKQALAFERTDFDQVRTLMENSDHCQDIRNLAFLGVAYNTLLRISEIARIRVKDISRTDSGRMLIHIGRTKTLVSTAGVEKALSLGVTELVERWISVSGVADDPNNYLFCRVRRYGVAKPSATCQLSTYALQGIFGAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVPIPEIMQAGGWTKVESVMNYIRNLDSETGAMVRLLEDGD

SEQ ID NO: 194 - protein, 485 aa, synthetic construct
MSDLQTLHQNLYALLADATCDEVRKNLMDMFRDHQAFSEHTWKMLLSVCRLWSAWCKLNNREWFPAEPEDVRDYLLHLQARGLAVKTIQHHLGSLNMLHRRAGLPRPGDSNAVSLVMRRIRKENVDAGERAKQALAFGRTDFDQVRSLMENSDRCQDIRNLAFLGVAYNTLLRISEIARIRVRDISRTDGGRMLIHIGRTKTLVSTAGVEKALSLGVTKLVERWISVSGVADDPNNHLFCRVRRYGVAKPSATSQLSTYALQGIFGAAHRLAYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPTGQKYLAWSGHSARVGAARDMARAGVSMAEIMQAGGWTTVNSVMNYIRNLDSETGAMVRLLEDGD

SEQ ID NO: 195 - protein, 485 aa, synthetic construct
MSNLQTLHQNLSALLVDATSDEARKNLMGVFRDRQAFSEHTWKTLLSVCRSWAAWCRLNNREWFPAEPEDVRDYLLHLQVRGLTVNTIQRHLALLSMLHRRSGLSRPGDSNAVSLVMRRIRKENVDAGERVKQALAFERTDFDQVRSLMENSDRCKDIRNLAFLGVAYNTLLRISEIARIRIKDISRTDGGRMLIHIGRTKTLVSTAGAEKALSLGVTRLVERWISVSGVADDPNNYLFCRVRKNGVAVPSATSLLSTDVLRGIFVAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 196 - protein, 484 aa, synthetic construct
MSNLLTLHDLFASLVDTTSDEARKNLMDLFRDRQAFSEHTWKMLMSVCRSWVAWCELNNRRWFPAEPEDVRDYLLYLQARGLAVKTIQHHLGSLNMLHRRAGLSRPGDSNAVPLVMRRIRRENVDAGERAKQALAFERTDLDRVRSLMENSDRCQDIRNLAFLGVAYNTLLRISEIARIRVRDISRTDGGRMLIHIGRTKTLVSTAGVEKALSLGVTKLVERWISVSGVADDPNNYLFCRVRRYGIAKPSATSQLSTYALQGIFGAAHRLVYGTRDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPPGQKYLAWSGHSARVGAARDMARAGVSIAEIMQAGGWTTVSSVMNYIRNLDSETGAMVRLLEDGE

SEQ ID NO: 197 - protein, 485 aa, synthetic construct
MSNLQTLHQNLSALLVDATSDEARKSLMDVFRDRQAFSEATWKTLLSVCRSWAAWCKSNNREWFPAEPEDVRDYLLHLQVRGLAVNTVQRHLALLSMLHRRSGLPRPGDSNAVSLVMRRIRKGNVDAGERVKQALAFERTDLDQVRSLMESSDRCKDIRNLALLGVAYNTLLRISEIARIRIKDISRTDGGRMLIHIGRTKTLVSTAGVEKALSLGVTGLVERWISVSGVADDPDNYLFCRVRKNGVAVPSATSQLTTDVLRGIFAAAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 198 - protein, 485 aa, synthetic construct
MSDLQALHQSLSALLTDVTSDEVRKNLMNMFRDRQAFSEHTWKMLLSVCRPWAAWCESNSREWFPADPEDVRDYLLHLQARGLAVKTIQHHLGSLNMLHRWSGLPRPGDSNAVSLVMRRIRRENVDAGERAKQALAFERTDFDRVRSLMENSDRCQDIRNLAFLGVAYNTLLRIGEIARIRVRDISRTDGGRMLIHIGRTKTLVSTAGVEKALSLGITKLVERWITVSGVADNPDNYLFCRVRRYGVAKPSATSQLSTYALQGIFGAAHRLVYGAKDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQKYLAWSGHSARVGAARDMARAGVSMPEIMQAGGWTTVNSVMNYIRNLDSETGAMVRLLEDDD

SEQ ID NO: 199 - protein, 485 aa, synthetic construct
MSNIQTLHQNLSALLVGATSDEARKSLMDVFRDRQAFSEATWKTLLSVCRSWAAWCELNDRNWFPAEPGDVRDYLLHLQARSLAVSTIQRHLALLNMLHRRSGLPRPGDSNAVSLVMRRIRKENVDAGERVKQALAFERTDFDQVRSLMENSDRCKDIRNLAFLGVAYNTLLRISEIARIRIKDISHTDGGRMLIHIGRTKTLVSTAGVEKALSLGVTRLVGRWISVSGVAGDPNNYLFCRVRKNGVAAPSATSQLSTDVLRGIFAAAHRLVYGARDGTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 200 - protein, 485 aa, synthetic construct
MSNLQTLHQNLTALLVGATSDEARKSLMDVFRDRQAFSEATWKTLLSVCRSWAAWCKSNNRRGLPAEPENVRDYLLHLQVRGLAVNTVQRHLALLNMLHRRSGLPRPGDSDAVSLVMRRIRKENVDAGERVKQALAFERTDLDQVRSLMENSDRCKDMRNLAFLGVAYNTLLRISEIARIRVGDISRTDGGRMLVHIGRTKTLVSTAGVEKALSLGVTRLVERWISASGVAGDPNNYLFCRVRKNGVAVPSATSQLSTDVLRGIFATAHRLVYGARDDTSAGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGPSGQRYLTWSGHSARVGAARDMARAGVSIAEIMQAGGWTTVESVMSYLRNLDSETGAMVRLLEDGD

SEQ ID NO: 201 - protein, 343 aa, unidentified
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 202 - protein, 12 aa, synthetic construct
GGSGGSGGSGGS

SEQ ID NO: 203 - protein, 24 aa, synthetic construct
GGSGGSGGSGGSGGSGGSGGSGGS

SEQ ID NO: 204 - protein, 452 aa, unidentified
MKAAIYIRVSTQEQVENYSIQAQTEKLTALCRSKDWDVYDIFIDGGYSGSNMNRPALNEMLSKLHEIDAVVVYRLDRLSRSQRDTITLIEEYFLKNNVEFVSLSETLDTSSPFGRAMIGILSVFAQLERETIRDRMVMGKIKRIEAGLPLTTAKGRTFGYDVIDTKLYINEEEAKQLQLIYDIFEEEQSITFLQKRLKKLGFKVRTYNRYNNWLTNDLYCGYVSYKDKVHVKGIHEPIISEEQFYRVQEIFTRMGKNPNMNRDSASLLNNLVVCSKCGLGFVHRRKDTMSRGKKYHYRYYSCKTYKHTHELEKCGNKIWRADKLEELIINRVNNYSFASRNVDKEDELDSLNEKLKIEHAKKKRLFDLYINGSYEVSELDSMMNDIDAQINYYESQIEANEELKKNKKIQENLADLATVDFDSLEFREKQLYLKSLINKIYIDGEQVTIEWL

SEQ ID NO: 205 - protein, 500 aa, unidentified [sequence omitted]

SEQ ID NO: 206 - protein, 500 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSVRYLQQLVHWAEDHKKLVVSATEAHFDTTTPFAAVVIALMGTVAQMELEAIRERSRSAAHFNIRAGKYRGSLPPWGYLPTRVDGEWRLVPDPVQRERILEVYHRVVDNHESLHRVAHDLNRRGVLSPKDYFAQLRGREPQGRKWSATALKCSLTSEAMLGYATLNGKTVRNDDGAPLVRAEPILTREQLEALRAELVKTSRAKPAVSTPSLLLRVLFCAVCGEPAYKFTGGGRKHPRYRCRSMGFPEHCGNGTVVMAEWDAFCEEQVLGLLEDAERLEKVWVAGSDSAVELAEVNAELVDLTSLIGSPAYRAGSPQREALDARIAALAARQGELEGLEARPSGWEWRATGQRFGDWWREQGTAAKNAWLRSMNVRLTFDVRGGLTRTIDFGDLREYEQHLRSGSVVDRLHTRMP

SEQ ID NO: 207 - protein, 500 aa, synthetic construct
MRALVVIRLSRVTDATTSPERQLESCQQLCTQRGWDVVGVAEDLDVSGAVDPFDCKRRPNLARWLAFEEQPFDVIVAYRVDRLTRSVRHLQQLVHWAEDHKKLVVSATEAHFDTTTPFAAVVIALMGTVAQMELEAIKERSRSAAHFNIRAGKYRGSLPPWGYLPTRVDGEWRLAPDPVQRERILEVYHRVVDNHEPLHLVAHDLNRRGVLSPKDYFAQLRGREPQGRKWSATALKRSMISEAMLGYATLNGKTVRGDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRGTGQRFGDWWREQDTAAKNTWLRSMNVRLTFDVRGGLTRTIDLGDLQEYEQHLRLGSVVERSHTGMP

SEQ ID NO: 208 - protein, 500 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSVRHLQQLVHWAEDHKKLVVSATEAHFDTTTPFAAVVIALMGTVAQMELEAIRERSRSATHFNIRAGKYRGSLPPWGYLPTRVDGEWRLVPDPVQRERILEVYHRVVDNHEPLHLVAHDLNRRGVLSPKDYFAQLRGRKPQGRKWSATALKRSMISEAMLGYATLNGKTVRDDDGAPLVRAEPILTREQQEALRVELVKTSRAKPAVSAPSLLLRVLFCAVCGEPAYKFAGGGRKHPRYRCRSMGFPKHCGNGTVAMAEWDAFCEEQVLNLLGDAERLEKVWVAGSDSAVELAEVNAELVDLTSLIGSPAYRAGSPQREALDTRIAALAARQEELEGLEARPSGWEWRGTGQRFGDWWREQDTAAKNTWLRSMNVRLAFDVRGGLTRTIDVGDLREYGQHLRLGSVVERLRTGMS

SEQ ID NO: 209 - protein, 500 aa, synthetic construct
MRALVVIRLSRVTDATTSPERQLESCQQLCAQRGWDVVGVAEDLDVSGAVDPFDRMRRPSLARWLAFEEQPFDVIVAYRVDRLTRSVRHLQQLVHWAEDHKKLVVSATEAHFDTTTPFAAVVIALMGTVAQMELEAIKERIRSAVRFNIRAGKYRGSLPPWGYLPTRVDGEWRLVPDPAQRERILEVYHRVVDNHEPLHLVAHDLNRRGVLSPKDYFAQLQGREPQGRKWSATALRRSMISGAMLGYATLNGKTVRGDDGAPLVRAEPILTREQQEALRAELAKTSRAKPAVSSPSLLLRVLFCAVCGEPAYKFVGGGRKHPRYRCRSMGSPKHCGNGTVVVAEWDAFCEEQVLDLLGDVERLEKVWVAGSDFAVELAEVNAELVDLTSLIGSPAYRAGSPQREALDARIAALAARQEELEGLEARPSGWEWRETGQRFGDWWREQDTAAKNTWLRSMNVRLTFDARGGLTRTIDFGDLQEYEQHLRLGGVVERLHTGMS

SEQ ID NO: 210 - protein, 500 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSVRHLQQLVHWAEDHKKLVVSATEAHFDTTTPFAAVVIALMGTVAQMELEAIKERNRSAAHFNIRAGKYRGSLPPWGYLPTRVDGEWRLAPDPVQRERILEVYHRVVNNHEPLHLVAHDLNRRGVLSPKDYFAQLQGREPQGRKWSATALKRSMISEAMLGYATLNGKTVRDDGGAPLVRAEPILTRGQLEALRAELAKTSRAKPAVSTPSLLLRVLFCAVCGEPAYKFAGGGRKHPRYRCRSMGFPKHCGNGTVVMAEWDAFCEEQVLDLLGDAERLEKVWITGSDSAVELAEVSAELVDLTSLIGSPAYRAGSPQREALDARIAALAARQEELEGLEVRPSGWEWRETGQRFGDWWREQDTAAKNTWLRSMNVRLTFDVRGGLTRTIDFGDLQEYEQHLRLGSVVERLHTGMS

SEQ ID NO: 211 - protein, 613 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 212 - protein, 637 aa, synthetic construct
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGGLTRTIDFGDLQEYEQHLRLGSVVERLHTGMS

SEQ ID NO: 213 - protein, 637 aa, synthetic construct
MRALVVIRLSRVTDATTSPERQLESCQQLCTQRGWDVVGVAEDLDVSGAVDPFDCKRRPNLARWLAFEEQPFDVIVAYRVDRLTRSVRHLQQLVHWAEDHKKLVVSATEAHFDTTTPFAAVVIALMGTVAQMELEAIKERSRSAAHFNIRAGKYRGSLPPWGYLPTRVDGEWRLAPDPVQRERILEVYHRVVDNHEPLHLVAHDLNRRGVLSPKDYFAQLRGREPQGRKWSATALKRSMISEAMLGYATLNGKTVRGDDGAPLVRAEPILTREQLEALRAELVKTGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRGTGQRFGDWWREQDTAAKNTWLRSMNVRLTFDVRGGLTRTIDLGDLQEYEQHLRLGSVVERSHTGMP

SEQ ID NO: 214 - protein, 637 aa, synthetic construct
MRALVVIRLSRVTDATTSPERQLESCQQLCTQRGWDVVGVAEDLDVSGAVDPFDCKRRPNLARWLAFEEQPFDVIVAYRVDRLTRSVRHLQQLVHWAEDHKKLVVSATEAHFDTTTPFAAVVIALMGTVAQMELEAIKERSRSAAHFNIRAGKYRGSLPPWGYLPTRVDGEWRLAPDPVQRERILEVYHRVVDNHEPLHLVAHDLNRRGVLSPKDYFAQLRGREPQGRKWSATALKRSMISEAMLGYATLNGKTVRGDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRGTGQRFGDWWREQDTAAKNTWLRSMNVRLTFDVRGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSGGLTRTIDLGDLQEYEQHLRLGSVVERSHTGMP

SEQ ID NO: 215 - protein, 637 aa, synthetic construct
MRALVVIRLSRVTDATTSPERQLESCQQLCTQRGWDVVGVAEDLDVSGAVDPFDCKRRPNLARWLAFEEQPFDVIVAYRVDRLTRSVRHLQQLVHWAEDHKKLVVSATEAHFDTTTPFAAVVIALMGTVAQMELEAIKERSRSAAHFNIRAGKYRGSLPPWGYLPTRVDGEWRLAPDPVQRERILEVYHRVVDNHEPLHLVAHDLNRRGVLSPKDYFAQLRGREPQGRKWSATALKRSMISEAMLGYATLNGKTVRGDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRGTGQRFGDWWREQDTAAKNTWLRSMNVRLTFDVRGGLTRTIDLGDGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSLQEYEQHLRLGSVVERSHTGMP

SEQ ID NO: 216 - protein, 637 aa, synthetic construct
MRALVVIRLSRVTDATTSPERQLESCQQLCTQRGWDVVGVAEDLDVSGAVDPFDCKRRPNLARWLAFEEQPFDVIVAYRVDRLTRSVRHLQQLVHWAEDHKKLVVSATEAHFDTTTPFAAVVIALMGTVAQMELEAIKERSRSAAHFNIRAGKYRGSLPPWGYLPTRVDGEWRLAPDPVQRERILEVYHRVVDNHEPLHLVAHDLNRRGVLSPKDYFAQLRGREPQGRKWSATALKRSMISEAMLGYATLNGKTVRGDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRGTGQRFGDWWREQDTAAKNTWLRSMNVRLTFDVRGGLTRTIDLGDLQEYEQHLRLGGGSGGSGGSGGSGGSGGSGGSGGSERPYACPVESCDRRFSRSDELTRHIRIHTGQKPFQCRICMRNFSRSDHLTTHIRTHTGEKPFACDICGRKFARSDERKRHTKIHLRQKDGGSGGSGGSGGSGGSGGSGGSGGSSVVERSHTGMP

SEQ ID NO: 217 - DNA, 56 bp, unidentified
  source 1..56: mol_type = genomic DNA
tgtttagttcctcgttttctctcgttggaagaagaagaaacgagaaactaaaatta

SEQ ID NO: 218 - DNA, 52 bp, unidentified
  source 1..52: mol_type = genomic DNA
gtggtttgtctggtcaaccaccgcggtctcagtggtgtacggtacaaaccca

SEQ ID NO: 219 - DNA, 48 bp, unidentified
  source 1..48: mol_type = genomic DNA
cgtttgtctggtcaagcaccgatgcttcagtgctgtacggtacaaacg

SEQ ID NO: 220 - DNA, 68 bp, synthetic construct
  source 1..68: mol_type = other DNA
acgcccacgcatgacggcttgtcgacgacggcggtctccgtcgtcaggatcatgcattgcgtgggcgt

SEQ ID NO: 221 - DNA, 68 bp, synthetic construct; identical to SEQ ID NO: 220
  source 1..68: mol_type = other DNA

SEQ ID NO: 222 - DNA, 68 bp, synthetic construct
  source 1..68: mol_type = other DNA
acgcccacgcgacgtggtttggtcactatggtcgtatctatagtgaagacctgtcgacgcgtgggcgt

SEQ ID NO: 223 - DNA, 78 bp, synthetic construct
  source 1..78: mol_type = other DNA
acgcccacgcgacgtggtttggctggtcaaccatcgtcgtatcaatggtgtacggtccaaacctcgacgcgtgggcgt

SEQ ID NO: 224 - DNA, 68 bp, synthetic construct
  source 1..68: mol_type = other DNA
gcgtgggcgtgacgtggtttggtcactatggtcgtatctatagtgaagacctgtcgacacgcccacgc

SEQ ID NO: 225 - DNA, 78 bp, synthetic construct
  source 1..78: mol_type = other DNA
gcgtgggcgtgacgtggtttggctggtcaaccatcgtcgtatcaatggtgtacggtccaaacctcgacacgcccacgc

SEQ ID NO: 226 - DNA, 38 bp, synthetic construct
  source 1..38: mol_type = other DNA
ggtttggtcactatggtcgtatctatagtgaagacctg